(19) United States
(12) Patent Application Publication
(10) Pub. No.: US 2026/0350784 A1
(43) Pub. Date: Nov. 5, 2026

(54) METHODS FOR DETERMINING LESION SIZE AND FITNESS POTENTIAL OF TISSUE

(71) Applicant: Regenall Limited, London (GB)

(72) Inventors: Mohamed Hamza NOORDEEN, London, Greater London (GB); Andrew TING, Kuala Lumpur (MY)

(21) Appl. No.: 19/122,377

(22) PCT Filed: Oct. 19, 2023
(86) PCT No.: PCT/GB2023/052718

§ 371 Date: Apr. 18, 2025

(30) Foreign Application Priority Data
Oct. 19, 2022 (GB) 2215448.8
Nov. 17, 2022 (GB) 2217225.8
Nov. 17, 2022 (GB) 2217228.2

(51) Int. Cl. G06T 7/00; A61B 8/08; G06T 7/62

(52) U.S. Cl. CPC G06T 7/0012; A61B 8/085; G06T 7/62; A61B 2503/40; G06T 2207/10132; G06T 2207/30048; G06T 2207/30096; G06T 2207/30101

(57) ABSTRACT

Methods for determining a size of a lesion, or for monitoring a change in a size of a lesion, in a tissue are described. Also described are methods for determining the fitness potential, or for monitoring a change in the fitness potential, of a tissue. Use of the methods for connective tissue or muscle tissue, particularly tendon, ligament, or cardiac tissue, is described. The methods may be implemented on a computer.

This invention relates to methods for determining the size of a lesion in a tissue (in particular, a connective tissue or a muscle tissue) in a subject, and to methods for determining a change in the size of a lesion in a subject, for example a tendon or ligament in a horse, or a tendon or ligament in a human, or a muscle, such as a cardiac muscle (for example, in a human). The methods may be computer implemented.

This invention also relates to methods for determining fitness potential of a tissue (in particular, a connective tissue or a muscle tissue) in a subject, and to methods for determining a change in the fitness potential of a tissue (in particular a connective tissue or a muscle tissue) in a subject, for example a tendon or a ligament in a horse, or a tendon or a ligament in a human, or a muscle, such as a cardiac muscle (for example, in a human). The methods may be computer implemented.

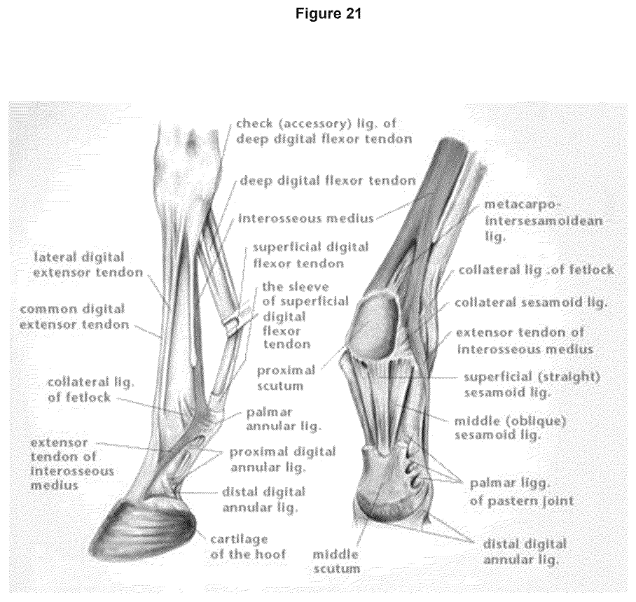

The equine limb contains several tendons and even more ligaments (see FIG. 21). Tendons attach muscles to bones, while ligaments attach bones to bones. Tendons are often named for their function. For example the superficial digital flexor tendon (SDFT) runs along the back of the limb extending from the knee to where it attaches on the pastern bones. It has many functions, but one of them is to flex the lower limb. The extensor tendons are on the front of the limb and they extend the limb. The suspensory ligament is one of the most important ligaments in the limb. It extends from the back of the knee or hock to the sesamoid bones, which are located in the back of the fetlockjoint. Many ligaments are quite short, such as the collateral ligaments, which act to help stabilize almost every joint.

In humans, tendon and ligament damage caused by injury or overuse, or associated with ageing and arthritis, is a common clinical problem. Damaged connective tissue heals very slowly and rarely recovers original tissue architecture and complete function. The Achilles tendon is the most frequently ruptured tendon in humans, despite being the thickest tendon in the human body. The Achilles tendon connects the back of the calf (gastrocnemius) muscle (as well as the soleus muscles) to the heel bone (the calcaneus bone). Another frequently damaged connective tissue is the anterior cruciate ligament (ACL), which is essential for knee kinematics, especially in rotation, and functions as an anterior/posterior stabiliser of the knee.

Tendons and ligaments are complex structures. They are made up of thousands of complex structures called collagen fibrils. The collagen fibrils are connected to other fibrils to make a collagen fibre. Fibres are bound together to make a fascicle. Small fascicles are bound together to make ever-larger groups of fascicles to form a tendon. This very structured internal organization is what gives a tendon its unique strength and function. Collagen is the most common molecule in tendons, but they also contain other molecules such as proteoglycans. The collagen and proteoglycans make up the scaffold of the tendon. Seventy percent of the weight of a tendon is water. Most of a tendon's ability to stretch comes from sliding of fascicles past one another. A small amount of a tendon's ability to stretch comes from the collagen molecules themselves stretching. The course of a tendon is not always straight. For example, the deep digital flexor tendon starts just behind the knee and then makes its way around the back of the fetlock before attaching to the foot. When tendons cross a bony prominence, such as a joint, they are usually encased within a tendon sheath. The tendon sheath contains synovial fluid, which is almost identical to the fluid contained within joints. This helps ease friction as the tendon slides over the bone point or over a joint. The digital flexor tendon sheath on the back of the fetlock joint helps ease the passage of the deep digital flexor tendon past the fetlock joint.

Tendons and ligaments have a variety of functions. They transmit forces to enable movement, support the lower limb, store energy, and provide support to joints. Tendons are viscoelastic, meaning that they have different material properties as different forces are applied to them. As force is first applied to a tendon it stretches quite a bit as the fascicles themselves stretch out. As more force is applied to the tendon, the fascicles start to slide past each other and the amount of stretch per unit of force is less than it was initially. Thus, the tendon is stiffer. This predictable relationship of increased force resulting in increased tendon stretch occurs until the load becomes too great and the tendon structures start to break down. This is called the yield point. Once this occurs there is irreparable damage done to the tendon and a tendon injury occurs.

There are two main types of tendon and ligament injuries; external injuries and overstrain injuries. External injuries include kicks, overreaching injuries, and lacerations. Overstrain injuries occur in two main ways. One is a sudden large overload of a tendon that was previously normal. The second type of injury is more common. With this type of injury there is a chronic, gradual build-up of tendon degradation or microtrauma. This damage builds up silently with no outward signs until the damage is too much and the tendon suddenly fails resulting in a significant injury.

Tendon and ligament injuries result in disruption of the highly organized internal organization of these structures. Initially there is haemorrhage or bleeding within the tendon or ligament. This is followed by a period of significant inflammation. Blood flow increases to the area, oedema or swelling occurs, and white blood cells congregate in the injury site. This is seen externally as an enlarged, hot, and painful tendon or ligament. This is the first stage of tendon or ligament repair and is designed to remove dead tendon fibres and cells, however the inflammation can get out of hand and result in even more damage. Treatment during this phase is directed at reducing the inflammation by doing things such as icing or cold hosing or administering anti-inflammatory drugs such as phenylbutazone. The inflammatory phase is short, lasting several days and overlaps with the next stage of tendon healing, which is the reparative phase. During this phase, new tissue is laid down within the tendon. This scar tissue has a different composition than normal tendon tissue. One of the largest differences is the type of collagen. Normal uninjured tendon has a predominance of Type I collagen. The new tissue laid down after an injury has a predominance of Type III collagen and is essentially scar tendon tissue. These different collagens have different structural properties meaning that the new tendon is not a strong as the old tendon.

The next phase of tendon healing, the remodelling phase, can last up to 18 months after injury. During this phase, the amount of Type I collagen gradually increases, but never returns to its original concentration. The body is able to repair the injury, but the scar tissue is never the same as the original tendon tissue. It is stiffer than normal tendon (does not stretch as much when loaded) and is more prone to re-injury.

Horses are particularly susceptible to connective tissue injuries, especially tendon and ligament injuries. Tendon and ligament injuries are reported to comprise up to almost half of sport horse injuries. Superficial digital flexor tendon (SDFT) injuries are reported to be the most common type of tendon/ligament injury. In one study of British racehorses and National Hunt horses, SDFT injuries accounted for up to 90% of tendon/ligament injuries.

The deep digital flexor tendon (DDFT) arises from three locations in the upper forelimb: the humerus, radius, and ulna. It then courses down the carpal canal (the depression running down the back of the knee) and crosses over the navicular bone before inserting at the back of the coffin bone, lying deep beneath the SDFT and just over the suspensory ligament. In the hind limb, the DDFT originates from two areas of the tibia and also inserts into the coffin bone. This tendon plays a role in the knee and forefoot flexion, forelimb elbow joint extension, and hock and hindfoot flexion and extension. Horse tendon injury occurs most frequently within the hoof capsule and the sheath around the tendon, likely from repetitive excessive loading. Usually, lesions appear in the body or borders of the tendon at the fetlock joint level and are more common in the hind than forelimbs. The four most common DDFT lesions are tendon enlargements or changes in shape, focal core lesions, mineralizations, and marginal tears.

Suspensory ligaments (SL) originate from the back of the fore and hind cannon bones. The SL's main function is to prevent the fetlockjoint from overextending. Although the suspensory ligament can fail at any point along its length, proximal (upper) suspensory desmitis (PSD) is often diagnosed in performance horses' limbs. It usually causes acute onset lameness that can resolve within 24 hours unless the horse continues to work hard. Lameness is typically mild to moderate. Although hind-limb PSD occurs in horses of all ages and disciplines, it is especially common in high-level dressage horses. Nerve blocks and ultrasonography are used to diagnose this injury. Prognosis for hind-limb PSD following conservative therapy alone is poor, with only 14% of horses resuming full work without lameness for more than a year.

In humans, the ACL and Achilles tendon are commonly injured. The ACL does not heal when torn, and surgical reconstruction is the standard treatment in the field of sports medicine. The ACL is divided into two parts, the anteromedial bundle (AMB) and the posterolateral bundle (PLB), which attach the femur to the tibia. The AMB is functional (tight) at knee flexion, and is moderately lax at the extended knee. Conversely, the PLB is functional at the extended knee, and lax at flexion. In this way, the AMB and PLB cooperate to control knee dynamics, particularly in rotation and tibia translation (preventing tibial overtranslation). Damage to the ACL is relatively immobilising for a subject, because at any point in the range of motion of the knee, one of the AMB or PLB is functional (tight), and thus the ACL is crucial to any motion about the knee. The Achilles tendon is susceptible to damage with repetitive use or overload, typically during sports. The tendon provides distal attachment sites for the calf muscle and soleus muscles, and inserts onto the posterior surface of the heel (calcaneus) bone. The plantaris tendon also fuses with the medial side of the Achilles tendon proximal to its attachment site.

Ultrasound is currently used to diagnose and monitor tendon and ligament injuries in horses (Leshaw, The Horse, 13 Oct. 2021: “Ultrasonography's Role in Equine Lameness Cases”), and is sometimes used to demonstrate connective tissue damage in humans, especially in athletes and sportsmen and sportswomen. Ultrasound uses high-frequency sound waves to produce images in real time. The user holds a sound-wave-emitting probe against the skin toward the structure being evaluated. When the waves meet structures or interfaces between structures, they reflect back to the probe like a ship's sonar. The more abrupt the interface or dense the structure, the more waves reflected. The more sound waves received, the brighter the structure looks on-screen. The brightness is described in terms of echogenicity. For example, bone appears bright (echogenic), normal fluid is dark (nonechogenic), and all other structures show up somewhere between.

When tendons or ligaments are strained, their fibres can tear. The extent of tendon or ligament damage can be evaluated by its size, echogenicity, and fibre pattern. Tendon or ligament injuries result in an increase in size of the structure, quantified as an increase in cross-sectional area. In cases of significant disruption, changes in echogenicity and fibre pattern are observed. Normally, the “echotexture” or patterning of a tendon or ligament is homogenous (even throughout); a perpendicular view of a normal tendon shows a round or ovoid structure with uniform shading. A damaged tendon might appear round and bright (normal fibres) with a dark area within it. Dark regions represent fibre disruption, or voids, where no sounds waves reflect. Larger, centrally located regions of fibre disruption are commonly referred to as core lesions. Viewing this same region's longitudinal axis, with the probe along the length of the tendon or ligament, the normally long, linear fibres might appear short and choppy or be missing altogether. Abnormalities are not always as overt, and true injuries could be as subtle as small, dark linear striations or mildly abnormal edges. After injury or treatment, clinical and ultrasound examinations are carried out to assess healing. A decrease in cross-sectional area, increase in echogenicity, and improvement in fibre alignment are signs of healing in tendon and ligament injuries.

As above, it is common practice in the art to diagnose and monitor tendon and ligament injuries using ultrasonography. A medical practitioner holds a transducer against the surface of skin adjacent to the connective tissue to be examined in the subject to capture images of the connective tissue at points in time. A practitioner must then assess the captured images, and manually outline the lesion using basic computer aided tools. The size of the lesion can be determined manually, using the area of the outlined tissue on the image and a scale bar. In this way, the ultrasonograms can be used to manually determine the size of a lesion in a subject.

It is also common practice to use ultrasonography to diagnose and monitor muscle tissue injuries, particularly in athletes.

Whist ultrasound is extremely useful for diagnosing and monitoring tendon, ligament, and muscle injuries, determining the size of a lesion is time-consuming and requires human input. As such, many medical practitioners do not determine the size of a lesion as a matter of normal practice.

There is, therefore, a need for improved ultrasound methods for use in assessment of the diagnosis and monitoring of connective tissue and muscle tissue injuries.

Conversely, echocardiograms are not currently used to accurately determine the presence or size of a lesion in cardiac tissue in a subject. Echocardiograms are routinely used to show how the blood moves through the heart and heart valves. Current diagnostic technology used to detect for cardiac tissue damage includes electrocardiography. This process produces an electrocardiogram (ECG), a recording of the heart's electrical activity through repeated cardiac cycles. An electrogram of the heart is produced, which shows a graph of voltage versus time of the electrical activity of the heart using electrodes placed on the skin. These electrodes detect the small electrical changes that are a consequence of cardiac muscle depolarization followed by repolarization during each cardiac cycle (heartbeat). Changes in the normal ECG pattern occur in numerous cardiac abnormalities, including cardiac rhythm disturbances (such as atrial fibrillation and ventricular tachycardia), inadequate coronary artery blood flow (such as myocardial ischemia and myocardial infarction), and electrolyte disturbances (such as hypokalemia and hyperkalemia. Among other things, an ECG can be used to measure the rate and rhythm of heartbeats, the size and position of the heart chambers, the presence of any damage to the heart's muscle cells or conduction system, the effects of heart drugs, and the function of implanted pacemakers.

However, an ECG cannot be used to accurately determine the size of a lesion in cardiac tissue, or to determine a change in the size of a lesion in the tissue.

There is therefore also a need for improved methods for use in assessment of the diagnosis and monitoring of muscle tissue injuries, particularly in cardiac tissue.

The Applicant has recognised that computer implemented methods provide rapid information on the size of a tissue lesion, allowing for quick and accurate diagnosis and monitoring of tissue injuries. Such methods enable appropriate treatment of the lesion to be administered soon after diagnosis, facilitating healing and functional recovery of the tissue. Such methods are suitable for use with connective tissues, such as ligaments and tendons, and muscle tissue, such as cardiac tissue.

A second aspect of the invention relates to methods for determining fitness potential of tissue in a subject, and to methods for determining a change in the fitness potential of tissue in a subject. Particularly, the invention relates to methods for determining the fitness potential, and change in fitness potential, of connective tissue, such as a tendon or a ligament in a horse, or a tendon or a ligament in a human. The invention also particularly relates to methods for determining fitness potential, and change in fitness potential, of muscle tissue, such as cardiac tissue (for example, in a human). The methods may be computer implemented.

As above, it is common practice in the art to diagnose and monitor tendon and ligament injuries using ultrasonography. In normal practice, a medical practitioner holds a transducer against the surface of skin adjacent to the connective tissue to be examined in the subject to capture images of the connective tissue at points in time. A practitioner must then assess the captured images, and manually perform diagnostic evaluations on the captured images using basic computer aided tools. In this way, disease states are determined.

As above, whist ultrasound is extremely useful for diagnosing and monitoring tendon and ligament injuries, minor changes in settings of the equipment, or operator method, can result in inconsistencies of image quality. This can make accurate diagnosis and monitoring of such injuries more difficult. Additionally, manually performing diagnostics on the captured images is time-consuming and requires human input. As such, many medical practitioners do not determine the fitness potential of a connective tissue as a matter of normal practice.

There is, therefore, a need for improved ultrasound methods for use in assessment of the integrity and/or condition of connective tissue, including for diagnosis and monitoring of connective tissue injuries.

There is also a need for improved ultrasound methods for use in assessment of the integrity and/or condition of muscle tissue, including for diagnosis and monitoring of muscle tissue injuries. Particularly, there is a need for improved methods for diagnosis and monitoring of cardiac tissue injury.

The Applicant has recognised that computer implemented methods provide rapid information on the echogenicity of a tissue, allowing for quick and accurate diagnosis and monitoring of tissue injuries. Such methods enable appropriate treatment of a lesion to be administered soon after diagnosis, facilitating healing and functional recovery of the tissue. Such methods are suitable for use with connective tissues, such as ligaments and tendons, and muscle tissue, such as cardiac tissue.

### Lesion Size

According to a first aspect of the invention there is provided a method for determining a size of a lesion in a connective tissue, which comprises:
i) determining an area of the lesion from an ultrasonogram of an ultrasound scan carried out on the connective tissue, and determining a total area of the connective tissue from the ultrasonogram; and
ii) determining the area of the lesion as a proportion of the total area of the connective tissue.

A method of the invention may be implemented on a general purpose computer, such as a laptop computer, desktop computer, tablet computer, or smart phone as a computerized method. The computerized method may be implemented on a general purpose computer in software such as an application or app.

According to the invention there is provided a computerized method for determining a size of a lesion in a connective tissue, the computerized method comprising:
i) determining an area of the lesion from an ultrasonogram of an ultrasound scan carried out on the connective tissue, and determining a total area of the connective tissue from the ultrasonogram; and
ii) determining the area of the lesion as a proportion of the total area of the connective tissue.

Methods of the invention are advantageous because real-time information is provided regarding the severity of the lesion. This allows for rapid diagnosis and monitoring of the lesion at any one time point, and over the course of a period. Such methods enable appropriate treatment of the lesion to begin soon after diagnosis, which can be tailored to the severity of the lesion. In doing so, the tissue can heal and functionally recover quickly.

Determining the area of the lesion as a proportion of the total area of the connective tissue allows for normalisation of the size of the lesion across different connective tissues. As one can imagine, different connective tissues occupy different total areas, depending on location and function. Thus, the size of a lesion will have different severities depending on the total area of the connective tissue. For example, a large lesion will be more severe if it is contained in a small connective tissue than a larger connective tissue. Determining the area of the lesion as a proportion of the total area of the connective tissue allows direct comparison of the lesion size across different connective tissues. For instance, methods of the invention can be used to compare a particular treatment for connective tissue injury across multiple subjects and multiple connective tissues. The proportionality output provides a proxy for the determination of efficacy of said treatment, when the area of the lesion as a proportion of the total area of the connective tissue is determined over a period of treatment.

Determining the area of the lesion as a proportion of the total area of the connective tissue also minimises differences in measurements for the area of the lesion due to changes in settings of the equipment used to determine the echogenicity values, or to differences in operator method.

Determination of the area of the lesion or total area of the connective tissue, according to methods of the invention, is carried out by determining the cross-sectional area of the lesion or cross-sectional area of the connective tissue. As such, the respective areas can be determined from one ultrasound scan of the tissue.

Methods of the invention can also be carried out for multiple ultrasound scans of the tissue, wherein an ultrasound scan is taken at multiple points along the depth of the lesion and the connective tissue. This provides a rudimentary indication of the volume of the lesion and connective tissue.

Determination of the area of the lesion as a proportion of the total area of the connective tissue can be calculated by dividing the area of the lesion by the total area of the connective tissue. The resultant figure may also be multiplied by 100 to provide a percentage of total area of the connective tissue comprising the lesion.

Optionally, according to methods of the invention, if the area of the lesion comprises:
i) 0-15% of the total area of the connective tissue, determining that the lesion is mild;
ii) 16-25% of the total area of the connective tissue, determining that the lesion is moderate; or
iii) >25% of the total area of the connective tissue, determining that the lesion is severe.

The determination of the severity of the lesion above incorporates the cross-sectional area of the lesion as a proportion of the total area of the connective tissue occupied by the lesion. The figure may also incorporate the proportion of the volume of the lesion as compared with the total volume of the connective tissue.

Optionally, a treatment program is initiated for the subject which is appropriate for the size and/or severity of the lesion. Said treatment program may comprise administering to the subject anti-inflammatory medication, dietary supplementation, or stem-cell treatment, and/or cold therapy, rest, confinement, surgery, or a loading/working training regime. For example, if the lesion is determined to be mild, the subject may need to reduce the amount of force applied to the connective tissue, by reducing exercise applied to the connective tissue for a period of time. If the lesion is determined to be moderate, the subject may need to rest the connective tissue, apply cold therapy, and/or administer anti-inflammatory medication. However, if the lesion is determined to be severe, the subject may require surgery.

Optionally, a method of the invention further comprises repeating steps (i) and (ii) after a period to determine whether there is a change in the area of the lesion as a proportion of the total area of the connective tissue.

Optionally the period is at least one day, at least a week, at least two weeks, at least a month, at least six months, or at least twelve months.

If it is determined that the area of the lesion as a proportion of the total area of the connective tissue has decreased over the period, this may indicate that the lesion is healing.

If the lesion is determined to be healing, the treatment program may be amended such that it is more suitable for the lesion after healing. For instance, the subject may no longer need to rest the connective tissue, but continue to be administered anti-inflammatory medication.

If it is determined that the area of the lesion as a proportion of the total area of the connective tissue has increased over the period, this may indicate that the lesion is progressing.

If the lesion is determined to be progressing, the treatment program may be amended such that it is more suitable for the lesion after progression. For instance, the subject may require surgery.

According to the invention, there is provided a method for monitoring a change in a size of a lesion in a connective tissue, which comprises:
i) determining an area of the lesion from an ultrasonogram of an ultrasound scan carried out on the connective tissue, and determining a total area of the connective tissue from the ultrasonogram;
ii) determining the area of the lesion as a proportion of the total area of the connective tissue;
iii) repeating steps (i) and (ii) after a period; and
iv) determining whether the area of the lesion as a proportion of the total area of the connective tissue has changed.

A method of the invention may be implemented on a general purpose computer, such as a laptop computer, desktop computer, tablet computer, or smart phone as a computerized method. The computerized method may be implemented on a general purpose computer in software such as an application or app.

According to the invention, there is provided a computerized method for monitoring a change in a size of a lesion in a connective tissue, the computerized method comprising:
i) determining an area of the lesion from an ultrasonogram of an ultrasound scan carried out on the connective tissue, and determining a total area of the connective tissue from the ultrasonogram;
ii) determining the area of the lesion as a proportion of the total area of the connective tissue;
iii) repeating steps (i) and (ii) after a period; and
iv) determining whether the area of the lesion as a proportion of the total area of the connective tissue has changed.

Optionally, if it is determined that the area of the lesion as a proportion of the total area of the connective tissue obtained in step (iii) is less than that obtained in step (ii), determining that the lesion size has decreased over the period. This may indicate that the lesion is healing.

If the lesion is determined to be healing, the treatment program may be amended such that it is more suitable for the lesion after healing. For instance, the subject may no longer need to rest the connective tissue, but continue to be administered anti-inflammatory medication.

Optionally, if it is determined that the area of the lesion as a proportion of the total area of the connective tissue obtained in step (iii) is greater than that obtained in step (ii), determining that the lesion size has increased over the period. This may indicate that the lesion is progressing.

If the lesion is determined to be progressing, the treatment program may be amended such that it is more suitable for the lesion after progression. For instance, the subject may require surgery.

Optionally, if it is determined that the area of the lesion as a proportion of the total area of the connective tissue obtained in step (iii) is the same as that obtained in step (ii), determining that the lesion size has remained the same over the period. This may indicate that the lesion is stable, or that a treatment program isn't effective.

If the lesion size has remained the same over the period, the treatment program may be continued unamended, until the area of the lesion decreases as a proportion of the total area of the connective tissue.

Optionally, the period is at least one day, at least a week, at least two weeks, at least a month, at least six months, or at least twelve months.

Optionally a method of the invention further comprises carrying out the ultrasound scan of the connective tissue, and providing an ultrasonogram from the ultrasound scan.

Optionally the ultrasonogram is a cross-sectional ultrasonogram.

Optionally the ultrasonogram is a longitudinal ultrasonogram.

Optionally the area of the lesion is determined by grey scale analysis of the ultrasonograms.

Optionally the area of the connective tissue which does not form a part of the area of the lesion is healthy connective tissue.

Optionally the connective tissue is a tendon or ligament tissue.

Optionally the lesion is the result of an injury to the subject.

Optionally the injury is an acute injury.

Optionally the injury is a traumatic injury.

Optionally the injury is an overload injury.

Optionally the injury is a chronic injury.

Optionally the chronic injury is caused by long-term micro-trauma or degradation of the connective tissue.

Optionally the chronic injury is a caused by chronic inflammation about the tissue.

An acute injury may be an injury that is less than one month old. A chronic injury may be an injury that is greater than one month old.

Optionally the subject is a horse.

Optionally the subject is a polo horse.

Optionally the polo horse takes a right forelimb lead.

Optionally the subject is a racehorse.

Optionally the racehorse takes a right or a left forelimb lead.

Optionally the connective tissue comprises a flexor tendon or an extensor tendon.

Optionally the flexor tendon is a superficial digital flexor tendon (SDFT), or a deep digital flexor tendon (DDFT).

Optionally the extensor tendon is a lateral digital extensor tendon (LDET), or a common digital extensor tendon (CDET).

Optionally the connective tissue comprises a suspensory ligament, or a check ligament.

Optionally wherein the subject is a non-human subject.

Optionally the subject is a human subject.

Optionally the human subject is an athlete, a sportsman, or a sportswoman.

Optionally the human subject is a professional athlete, sportsman, or sportswoman.

The term “athlete” is used herein to include a person (male or female) who is trained or skilled in exercises, sports, or games requiring physical strength, agility, or stamina. Examples of athletes include sprinters (including runners of track races of 100 metres, 200 metres, and 400 metres), middle distance runners (including runners of track races of 500 metres to less than 3,000 metres, including 800 metres, 1500 metres), long distance runners (including runners of at least 3000 metres, including 3000 metres, 5000 metres, 10,000 metres), hurdles (including runners of track races of 100 metre hurdles, 110 metre hurdles, 400 metre hurdles) relays (4×100 metres, 4×400 metres). Examples of sports include association football (often referred to as simply “football” or “soccer”), rugby football (including rugby union or rugby league), American football, basketball, baseball (including Major League Baseball).

Optionally the connective tissue comprises an anterior cruciate ligament (ACL).

Optionally the connective tissue comprises an Achilles tendon.

Optionally the connective tissue comprises an extensor tendon.

Optionally the connective tissue comprises a flexor tendon.

Optionally the connective tissue comprises a tibial tendon.

Optionally the connective tissue comprises a peroneal tendon.

According to the invention there is also provided a method for determining a size of a lesion in a muscle tissue, which comprises determining an area of the lesion from an ultrasonogram of an ultrasound scan carried out on the muscle tissue.

A method of the invention may be implemented on a general purpose computer, such as a laptop computer, desktop computer, tablet computer, or smart phone as a computerized method. The computerized method may be implemented on a general purpose computer in software such as an application or app.

According to the invention there is provided a computerized method for determining a size of a lesion in a muscle tissue, the computerized method comprising determining an area of the lesion from an ultrasonogram of an ultrasound scan carried out on the muscle tissue.

Optionally the method further comprises determining the area of the lesion as a proportion of the total area of the muscle tissue.

Optionally a treatment program is initiated for the subject which is appropriate for the size and/or severity of the lesion.

Optionally the treatment program comprises administering to the subject anti-inflammatory medication, dietary supplementation, or stem-cell treatment, and/or cold therapy, rest, confinement, surgery, or a loading/working training regime.

Optionally the method is repeated after a period to determine whether there is a change in the area of the lesion.

Optionally the period is at least one day, at least a week, at least two weeks, at least a month, at least six months, or at least twelve months.

If it is determined that the area of the lesion has decreased over the period, this indicates that the lesion is healing. If it is determined that the area of the lesion has increased over the period, this indicates that the lesion is progressing.

There is also provided according to the invention a method for monitoring a change in a size of a lesion in a muscle tissue, which comprises:
i) determining an area of the lesion from an ultrasonogram of an ultrasound scan carried out on the muscle tissue;
ii) repeating step (i) after a period; and
iii) determining whether the area of the lesion has changed.

A method of the invention may be implemented on a general purpose computer, such as a laptop computer, desktop computer, tablet computer, or smart phone as a computerized method. The computerized method may be implemented on a general purpose computer in software such as an application or app.

According to the invention, there is provided a computerized method for monitoring a change in a size of a lesion in a muscle tissue, the computerized method comprising:
i) determining an area of the lesion from an ultrasonogram of an ultrasound scan carried out on the muscle tissue;
ii) repeating step (i) after a period; and
iii) determining whether the area of the lesion has changed.

If it is determined that the area of the lesion obtained in step (iii) is less than that obtained in step (ii), it is determined that the lesion size has decreased over the period. This indicates that the lesion is healing.

If it is determined that the area of the lesion obtained in step (iii) is greater than that obtained in step (ii), it is determined that the lesion size has increased over the period. This indicates that the lesion is progressing.

If it is determined that the area of the lesion obtained in step (iii) is the same as that obtained in step (ii), it is determined that the lesion size has remained the same over the period. This indicates that the lesion is stable.

Optionally the period is at least one day, at least a week, at least two weeks, at least a month, at least six months, or at least twelve months.

Optionally a method of the invention further comprises carrying out the ultrasound scan of the tissue, and providing an ultrasonogram from the ultrasound scan.

Optionally the ultrasonogram is a cross-sectional ultrasonogram.

Optionally the ultrasonogram is a longitudinal ultrasonogram.

Optionally the area of the lesion is determined by grey scale analysis of the ultrasonograms.

Optionally an area of the muscle tissue which does not form a part of the area of the lesion is healthy muscle tissue.

Optionally the muscle tissue is cardiac tissue.

Optionally the lesion is the result of an injury to the subject.

Optionally the injury is an acute injury.

Optionally the muscle tissue is cardiac muscle tissue, and the injury is caused by decreased or complete cessation of blood flow to the cardiac tissue.

Optionally the injury is caused by a full or partial occlusion of a blood vessel.

Optionally the blood vessel is a coronary artery.

Optionally the injury is caused by a tear or strain.

Optionally the muscle is cardiac muscle and the injury is caused by blunt cardiac injury.

Optionally the injury is a traumatic injury.

Optionally the injury is an overload injury.

Optionally the injury is a chronic injury.

Optionally the injury is caused by long-term disruption of blood flow to the tissue.

Optionally the tissue is cardiac tissue and the injury is caused by atherosclerosis.

Optionally the chronic injury is caused by long-term micro-trauma or degradation of the muscle tissue.

Optionally the chronic injury is caused by chronic inflammation about the tissue.

Optionally the muscle tissue is human muscle tissue.

Optionally the subject is a human subject.

An advantage of methods of the invention is that they can provide real-time information on lesion size. The methods also provide information on the change in the size and severity of the lesion over a period. This allows for rapid diagnosis and monitoring of a connective tissue lesion. Such methods enable appropriate treatment of the lesion to be administered soon after diagnosis, and for the treatment to be modified over a period of time depending on changes in the state of the lesion. Thus, methods of the invention facilitate healing and functional recovery of the tissue.

### Fitness Potential

According to a second aspect of the invention there is provided a method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue; and
ii) determining a ratio of the echogenicity value of the first connective tissue to the echogenicity value of the second tissue.

According to the invention there is also provided a method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a ratio of the echogenicity value of the first connective tissue to the echogenicity value of the second tissue; and
iii) if the ratio determined in step (ii) is:
   a. less than one, determining that the fitness potential of the connective tissue of interest is lower than the fitness potential of the second connective tissue; or
   b. greater than one, determining that the fitness potential of the connective tissue of interest is higher than the fitness potential of the second connective tissue; or
   c. one, determining that the fitness potential of the connective tissue of interest is the same as the fitness potential of the second connective tissue.

According to the invention there is also provided a method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a ratio of the echogenicity value of the first connective tissue to the echogenicity value of the second tissue; and
iii) if the ratio determined in step (ii) is:
   a. less than one, determining that the fitness potential of the connective tissue of interest is lower than the fitness potential of the second connective tissue; or
   b. greater than one, determining that the fitness potential of the connective tissue of interest is higher than the fitness potential of the second connective tissue.

Methods of the invention are advantageous because the ratio of the echogenicity value of the first connective tissue to the echogenicity value of the second connective tissue determined in step (ii) provides a normalisation of the echogenicity value of the first connective tissue. This minimises differences in echogenicity values obtained due to changes in settings of the equipment used to determine the echogenicity values, or to differences in operator method.

Methods of the invention allow, for example, more accurate determination of the fitness potential of the connective tissue of interest. The ratio determined at one time can also be compared with the ratio determined at a different time. This allows, for example, more accurate monitoring of changes in fitness potential of the connective tissue of interest.

The term “fitness potential” is used herein to refer to a measure of the integrity or condition of the connective tissue of interest. It can be used, for example, to provide a diagnosis or an assessment of an injury to the connective tissue of interest, or of the state of recovery or healing of an injury to the connective tissue of interest, or of the susceptibility of the connective tissue of interest to injury. It can also provide a measure of the strength of the connective tissue of interest, for example to monitor improvements to already healthy connective tissue during or after a training program.

Where the second connective tissue is healthy connective tissue, it is expected that the echogenicity value of that tissue will remain relatively constant, so any change in the ratio over time will be due to a change in echogenicity of the connective tissue of interest.

A method of the invention may be implemented on a general purpose computer, such as a laptop computer, desktop computer, tablet computer, or smart phone as a computerized method. The computerized method may be implemented on a general purpose computer in software such as an application or app.

According to the invention there is provided a computerized method for determining the fitness potential of a connective tissue of interest in a subject, the computerized method comprising:
i) determining an echogenicity value of a first connective tissue in a limb of the subject, and an echogenicity value of a second connective tissue in the same limb of the subject, wherein the connective tissue of interest is the first connective tissue;
ii) determining a ratio of the echogenicity value of the first connective tissue to the echogenicity value of the second tissue; and
iii) if the ratio determined in step (ii) is:
a) less than one, determining that the fitness potential of the connective tissue of interest is lower than the fitness potential of the second connective tissue; or
b) greater than one, determining that the fitness potential of the connective tissue of interest is higher than the fitness potential of the second connective tissue; or
c) one, determining that the fitness potential of the connective tissue of interest is the same as the fitness potential of the second connective tissue.

Optionally the second connective tissue is healthy connective tissue, and in step (iii), if the ratio determined in step (ii) is:
a. less than one, determining that the connective tissue of interest has a low fitness potential; or
b. greater than one, determining that the connective tissue of interest has a high fitness potential; or
c. one, determining that the connective tissue of interest is healthy.

If the connective tissue of interest has a low fitness potential, this may indicate, for example, that the connective tissue of interest is injured, or has a higher risk of injury.

If the connective tissue of interest has a high fitness potential, this may indicate, for example, that the connective tissue of interest is strong, or has a lower risk of injury.

Depending on the value of the ratio determined in step (ii), action can then be taken as appropriate. For example, if the ratio is less than one, the subject may need to rest that limb or suitable treatment may need to be administered for an injury. If the ratio is one, or greater than one, normal activity may be resumed or continued, or increased activity may be initiated (for example, a more active training program may be initiated).

Optionally a method of the invention further comprises repeating steps (i) to (iii) after a period to determine whether there is a change in the fitness potential of the connective tissue of interest.

Optionally the period is at least one day, at least a week, at least two weeks, or at least a month.

According to the invention there is also provided a method for determining a change in the fitness potential of a connective tissue of interest in a subject, the method comprising:
i) determining an echogenicity value of a first connective tissue in a limb of the subject, and an echogenicity value of a healthy second connective tissue in the same limb of the subject, wherein the connective tissue of interest is the first connective tissue;
ii) determining a ratio of the echogenicity value of the first connective tissue to the echogenicity value of the second tissue; and
iii) repeating steps (i) and (ii) after a period; and
iv) if the ratio obtained in step (iii) is:
   a. higher than the ratio obtained in step (ii), determining that the fitness potential of the connective tissue of interest has increased; or
   b. similar to the ratio obtained in step (ii), determining that the fitness potential of the connective tissue of interest has remained the same; or
   c. lower than the ratio obtained in step (ii), determining that the fitness potential of the connective tissue of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

According to the invention there is provided a computerized method for determining a change in the fitness potential of a connective tissue of interest in a subject, the computerized method comprising:
i) determining an echogenicity value of a first connective tissue in a limb of the subject, and an echogenicity value of a healthy second connective tissue in the same limb of the subject, wherein the connective tissue of interest is the first connective tissue;
ii) determining a ratio of the echogenicity value of the first connective tissue to the echogenicity value of the second tissue; and
iii) repeating steps (i) and (ii) after a period; and
iv) if the ratio obtained in step (iii) is:
   a. higher than the ratio obtained in step (ii), determining that the fitness potential of the connective tissue of interest has increased; or
   b. similar to the ratio obtained in step (ii), determining that the fitness potential of the connective tissue of interest has remained the same; or
   c. lower than the ratio obtained in step (ii), determining that the fitness potential of the connective tissue of interest has reduced.

Optionally the period is at least one day, at least a week, at least two weeks, or at least a month.

Optionally the connective tissue of interest, in a method for determining a change in the fitness potential of a connective tissue of interest in a subject, is an injured connective tissue, and if the ratio obtained in step (iii) is higher than the ratio obtained in step (ii), it is determined that the injury to the connective tissue of interest is healing.

Optionally the connective tissue of interest, in a method for determining a change in the fitness potential of a connective tissue of interest in a subject, is a healthy connective tissue, and if the ratio obtained in step (iii) is lower than the ratio obtained in step (ii), it is determined that the connective tissue of interest is at risk of injury (or has a higher risk of injury).

Optionally the first connective tissue is in a forelimb of the subject.

Optionally the first connective tissue is in a left forelimb of the subject.

Optionally the first connective tissue is in a right forelimb of the subject.

Optionally the first connective tissue is in a hindlimb of the subject.

Optionally the first connective tissue is in a left hindlimb of the subject.

Optionally the first connective tissue is in a right hindlimb of the subject.

It will be appreciated that, in other embodiments, instead of determining a ratio of the echogenicity value of the first connective tissue to the echogenicity value of the second connective tissue in step (ii), a ratio of the echogenicity value of the second connective tissue to the echogenicity value of the first connective tissue can instead be determined.

According to the invention there is provided a method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue; and
ii) determining a ratio of the echogenicity value of the second connective tissue to the echogenicity value of the first connective tissue.

A method of the invention may be implemented on a general purpose computer, such as a laptop computer, desktop computer, tablet computer, or smart phone as a computerized method. The computerized method may be implemented on a general purpose computer in software such as an application or app.

According to the invention there is also provided a method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a ratio of the echogenicity value of the second connective tissue to the echogenicity value of the first connective tissue; and
iii) if the ratio determined in step (ii) is:
   a. greater than one, determining that the fitness potential of the connective tissue of interest is lower than the fitness potential of the second connective tissue; or
   b. less than one, determining that the fitness potential of the connective tissue of interest is higher than the fitness potential of the second connective tissue; or
   c. one, determining that the fitness potential of the connective tissue of interest is the same as the fitness potential of the second connective tissue.

A method of the invention may be implemented on a general purpose computer, such as a laptop computer, desktop computer, tablet computer, or smart phone as a computerized method. The computerized method may be implemented on a general purpose computer in software such as an application or app.

According to the invention there is also provided a method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a ratio of the echogenicity value of the second connective tissue to the echogenicity value of the first connective tissue; and
iii) if the ratio determined in step (ii) is:
   a. greater than one, determining that the fitness potential of the connective tissue of interest is lower than the fitness potential of the second connective tissue; or
   b. less than one, determining that the fitness potential of the connective tissue of interest is higher than the fitness potential of the second connective tissue.

A method of the invention may be implemented on a general purpose computer, such as a laptop computer, desktop computer, tablet computer, or smart phone as a computerized method. The computerized method may be implemented on a general purpose computer in software such as an application or app.

Optionally the second connective tissue is healthy connective tissue, and in step (iii), if the ratio determined in step (ii) is:
a. greater than one, determining that the connective tissue of interest has a low fitness potential; or
b. less than one, determining that the connective tissue of interest has a high fitness potential; or
c. one, determining that the connective tissue of interest is healthy.

According to the invention there is also provided a method for determining a change in the fitness potential of a connective tissue of interest in a subject, the method comprising:
i) determining an echogenicity value of a first connective tissue in a limb of the subject, and an echogenicity value of a healthy second connective tissue in the same limb of the subject, wherein the connective tissue of interest is the first connective tissue;
ii) determining a ratio of the echogenicity value of the second connective tissue to the echogenicity value of the first connective tissue; and
iii) repeating steps (i) and (ii) after a period; and
iv) if the ratio obtained in step (iii) is:
   a. lower than the ratio obtained in step (ii), determining that the fitness potential of the connective tissue of interest has increased; or
   b. similar to the ratio obtained in step (ii), determining that the fitness potential of the connective tissue of interest has remained the same; or
   c. higher than the ratio obtained in step (ii), determining that the fitness potential of the connective tissue of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

According to the invention there is also provided a method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a relative difference between the echogenicity values obtained for the first and second connective tissues in step (i);
iii) comparing the relative difference obtained in step (ii) with a corresponding relative difference obtained for first and second connective tissue in a limb of the same or a different subject; and
iv) if the relative difference obtained in step (ii) is:
   a. lower than the compared relative difference in step (iii), determining that the fitness potential of the connective tissue of interest is lower than the fitness potential of the first connective tissue in the compared limb; or
   b. similar to the compared relative difference in step (iii), determining that the fitness potential of the connective tissue of interest is similar to the fitness potential of the first connective tissue in the compared limb; or
   c. higher than the compared relative difference in step (iii), determining that the fitness potential of the connective tissue of interest is higher than the fitness potential of the first connective tissue in the compared limb.

A method of the invention may be implemented on a general purpose computer, such as a laptop computer, desktop computer, tablet computer, or smart phone as a computerized method. The computerized method may be implemented on a general purpose computer in software such as an application or app.

Optionally the compared limb in step (iii) is a different limb of the same subject.

Optionally the compared limb in step (iii) is the same limb of a different subject.

Optionally the compared limb in step (iii) is a different limb of a different subject.

Optionally the corresponding relative difference obtained for first and second connective tissue of the compared limb in step (iii) is a predetermined corresponding relative difference previously obtained for the first and second connective tissues of the compared limb.

Optionally the corresponding relative difference obtained for first and second connective tissue in step (iii) is a predetermined corresponding relative difference previously obtained for the first and second connective tissues in the same limb of the same subject.

Optionally the predetermined corresponding relative difference was obtained when the connective tissue of interest was healthy.

Optionally the echogenicity value of the connective tissue of interest obtained in step (i) is obtained after an injury to the first connective tissue.

Optionally the predetermined corresponding relative difference was obtained after an injury to the connective tissue of interest.

Optionally the echogenicity value of the connective tissue of interest obtained in step (i) is obtained while the injury to the first connective tissue is healing.

Optionally the second connective tissue in step (i) is healthy connective tissue, and the first and second connective tissue in step (iii) is healthy connective tissue, and wherein if the relative difference obtained in step (ii) is:
a. lower than the compared relative difference in step (iii), determining that the connective tissue of interest is injured or at risk of injury; or
b. similar to the compared relative difference in step (iii), determining that the connective tissue of interest is healthy; or
c. higher than the compared relative difference in step (iii), determining that the connective tissue of interest has high fitness potential.

Optionally a method of the invention further comprises repeating steps (i) to (iii) after a period to determine whether there is a change in the fitness potential of the connective tissue of interest.

Optionally the period is at least one day, at least a week, at least two weeks, or at least a month.

According to the invention there is also provided a method for determining a change in the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a relative difference between the echogenicity values obtained for the first and second connective tissues in step (i);
iii) comparing the relative difference obtained in step (ii) with a corresponding relative difference obtained for first and second connective tissue in a limb of the same or a different subject;
iv) repeating steps (i) to (iii) after a period; and
v) determining whether there is a difference between the compared relative difference obtained in step (iii) and the compared relative difference obtained in step (iv); and if the compared relative difference obtained in step (iii) is:
   a. higher than the compared relative difference obtained in step (iv), determining that the fitness potential of the connective tissue of interest has improved; or
   b. similar to the compared relative difference obtained in step (iv), determining that the fitness potential of the connective tissue of interest has remained the same; or
   c. lower than the compared relative difference obtained in step (iv), determining that the fitness potential of the connective tissue of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

Optionally the period is at least one day, at least a week, at least two weeks, or at least a month.

Optionally the second connective tissue, in step (i) of a method for determining a change in the fitness potential of a connective tissue of interest in a subject, is healthy connective tissue, and the first and second connective tissue in step (iii) is healthy connective tissue.

Optionally the connective tissue of interest, in a method for determining a change in the fitness potential of a connective tissue of interest in a subject, is an injured connective tissue, and if the compared relative difference obtained in step (iii) is higher than the compared relative difference obtained in step (iv), it is determined that the injury to the connective tissue of interest is healing.

Optionally the connective tissue of interest, in a method for determining a change in the fitness potential of a connective tissue of interest in a subject, is a healthy connective tissue, and if the compared relative difference obtained in step (iii) is lower than the compared relative difference obtained in step (iv), it is determined that the connective tissue of interest is at risk of injury.

Optionally the connective tissue is tendon or ligament tissue.

Optionally the echogenicity values are determined quantitatively.

Optionally the echogenicity values are determined by ultrasonography.

Optionally the echogenicity values are determined by quantifying the echogenicity of ultrasonographic images.

Optionally the echogenicity is quantified by grey scale analysis of ultrasonographic images.

Optionally the grey scale analysis of ultrasonographic images includes analysis of cross-sectional ultrasonographic images taken of the connective tissues.

Optionally the grey scale analysis of ultrasonographic images includes analysis of longitudinal ultrasonographic images taken of the connective tissues.

Optionally a mean echogenicity value is determined for each connective tissue in each limb.

Optionally a quantitative measure of echogenicity of the connective tissue of interest and the second connective tissue is computed from an ultrasound image (an ultrasonograph) of each tissue. This can be performed by grey scale analysis using any suitable software, for example open-source image processing software, ImageJ2 (version 2.3.0/1.53f). A mean grey scale (MGS) value for each tissue is determined. Each tissue has a value between 0 (black) and 255 (white), representing the mean echogenicity value of the tissue. A ratio of the resultant values is determined for each limb (for example, an SDFT/DDFT ratio). The ratio of mean echogenicity of the injured tendon to that of the healthy tendon is determined to assess the extent of damage/healing at a lesion.

Optionally the connective tissue of interest comprises damaged tissue.

Optionally a mean echogenicity value is determined for a lesion in the damaged tissue.

Optionally a mean echogenicity value is determined for tissue excluding a lesion in the damaged tissue.

Optionally the damage tissue is the result of an injury to the subject.

Optionally the injury is an acute injury.

Optionally the injury is a traumatic injury.

Optionally the injury is an overload injury.

Optionally the injury is a chronic injury.

Optionally the chronic injury is caused by long-term micro-trauma or degradation of the connective tissue.

Optionally the chronic injury is caused by chronic inflammation about the tissue.

Optionally the subject is a non-human animal, Including for example, a domesticated pet, such as a dog, or a cat.

Optionally the first connective tissue is in a forelimb, or an upper limb, of the subject.

Optionally the first connective tissue is in a hindlimb, or a lower limb, of the subject.

Optionally the subject is a horse.

Optionally the subject is a polo horse.

Optionally the polo horse takes a right forelimb lead.

Optionally the horse is a racehorse.

Optionally the racehorse takes a right or a left forelimb lead.

Optionally the connective tissue comprises a flexor tendon or an extensor tendon.

Optionally the flexor tendon is a superficial digital flexor tendon (SDFT), or a deep digital flexor tendon (DDFT).

Optionally the first connective tissue is a SDFT and the second connective tissue is a DDFT.

Optionally the first connective tissue is a DDFT and the second connective tissue is a SDFT.

Optionally the extensor tendon is a lateral digital extensor tendon (LDET), or a common digital extensor tendon (CDET).

Optionally the first connective tissue is a LOET and the second connective tissue is a CDET.

Optionally the first connective tissue is a CDET and the second connective tissue is a LDET.

Optionally the first connective tissue comprises a suspensory ligament, or a check ligament.

Optionally the first connective tissue is a suspensory ligament and the second connective tissue is a check ligament.

Optionally the first connective tissue is a check ligament and the second connective tissue is a suspensory ligament.

Optionally the first connective tissue is a healthy connective tissue (i.e. does not comprise an injury).

Optionally the subject is a horse, wherein the first connective tissue is a healthy connective tissue (i.e. does not comprise an injury).

In some embodiments, the first connective tissue is not a SDFT of a horse.

In some embodiments, the first connective tissue is not a left fore SDFT of a horse.

In some embodiments, the first connective tissue is not a check ligament of a horse.

In some embodiments, the first connective tissue is not a left fore check ligament of a horse.

In some embodiments, the subject is a horse, excluding a polo horse.

Optionally the subject is a human subject.

Optionally the human subject is an athlete, a sportsman, or a sportswoman.

Optionally the human subject is a professional athlete, sportsman, or sportswoman.

The term “athlete” is used herein to include a person (male or female) who is trained or skilled in exercises, sports, or games requiring physical strength, agility, or stamina. Examples of athletes include sprinters (including runners of track races of 100 metres, 200 metres, and 400 metres), middle distance runners (including runners of track races of 500 metres to less than 3,000 metres, including 800 metres, 1500 metres), long distance runners (including runners of at least 3000 metres, including 3000 metres, 5000 metres, 10,000 metres), hurdles (including runners of track races of 100 metre hurdles, 110 metre hurdles, 400 metre hurdles) relays (4×100 metres, 4×400 metres). Examples of sports include association football (often referred to as simply “football” or “soccer”), rugby football (including rugby union or rugby league), American football, basketball, baseball (including Major League Baseball).

Optionally the first connective tissue is in an upper limb of the subject.

Optionally the first connective tissue is a tendon in a finger in a hand of the subject, and the second connective tissue is a tendon in a different finger in the same hand of the subject.

Optionally the first connective tissue is in a lower limb of the subject.

Optionally the first connective tissue is a tendon in a toe in a foot of the subject, and the second connective tissue is a tendon in a different toe in the same foot of the subject.

Optionally the first connective tissue comprises a tendon in a foot of the subject, wherein the tendon is selected from a tibial tendon, an Achilles tendon, or a peroneal tendon, and the second connective tissue comprises a different tendon in the same foot of the subject, wherein the tendon is selected from a tibial tendon, an Achilles tendon, or a peroneal tendon.

Optionally the first connective tissue comprises a posterior tibial tendon, and the second connective tissue comprises an anterior tibial tendon.

Optionally the first connective tissue comprises an anterior tibial tendon, and the second connective tissue is a posterior tibial tendon.

Optionally the second connective tissue is an equivalent connective tissue to the first connective tissue.

An equivalent connective tissue may be in the same limb as the connective tissue of interest.

The term “equivalent connective tissue” is used herein to refer to a connective tissue which has the same or a similar structure and/or function as the connective tissue of interest. The skilled person will appreciate that it is advantageous for an equivalent connective tissue to have the same or a similar tissue structure and composition to the connective tissue of interest, such that its echogenicity can be used as a suitable comparison to the echogenicity of the connective tissue of interest. The equivalent connective tissue can be used, for example, to provide a comparative connective tissue for the determination of the fitness potential of the connective tissue of interest. In particular, the equivalent connective tissue can be used for determination of a ratio of the echogenicity value of the connective tissue of interest to the echogenicity value of the equivalent connective tissue. This ratio provides an indication of the relative fitness potential of the connective tissue of interest. Additionally, the echogenicity value of the equivalent connective tissue can be used to determine the change in fitness potential of the connective tissue of interest over time. The echogenicity value of the equivalent connective tissue may provide a relatively fixed value such that the change in echogenicity value of the connective tissue of interest can be deduced. This in turn can be used to determine the amount and rate of healing of a damaged connective tissue, or the propensity for a connective tissue to become damaged over time. It can also provide a means to determine the strength of the connective tissue of interest, for example to monitor improvements to already healthy connective tissue during or after a training program.

A connective tissue of interest may be in an upper limb or a forelimb of the subject, or in a lower limb or a hindlimb of the subject. A connective tissue of interest may, for example, be in a shoulder, arm, upper arm, lower arm, elbow, wrist, hand, or finger, a hip, leg, upper leg, lower leg, knee, ankle, foot, or toe of the subject.

For example, in a human, the connective tissue of interest may be in a finger of one hand, and an equivalent connective tissue may be in a different finger (for example, an adjacent finger) of the same hand. Similarly, the connective tissue of interest may be in a toe of one foot, and an equivalent connective tissue may be in a different toe (for example, an adjacent toe) of the same foot. In specific examples in the foot, the connective tissue of interest may be the tibialias posterior, Achilles Tendon or the peroneal tendon, in humans. The equivalent tendon may, for example, be one of these tendons in the same foot.

The equivalent connective tissue may perform a similar function as the connective tissue of interest. For example, the connective tissue of intertest may be a flexor tendon, and the equivalent connective tissue may also be a flexor tendon. Alternatively, the connective tissue of intertest may be an extensor tendon, and the equivalent connective tissue may also be an extensor tendon. For example, the connective tissue of interest may be a flexor tendon (for example, a flexor tendon of a finger), and the equivalent connective tissue may also be a flexor tendon (for example, a flexor tendon of a different finger in the same hand, such as an adjacent finger), rather than an extensor tendon.

We have also appreciated that differences in echogenicity values obtained due to changes in settings of the equipment used to determine the echogenicity values, or to differences in operator method, can be minimised by comparing the echogenicity value of a connective tissue of interest in a limb of a subject with an equivalent connective tissue in another limb of the subject. Again, such methods allow, for example, more accurate determination of the fitness potential of the connective tissue of interest. The ratio determined at one time can also be compared with the ratio determined at a different time. This allows, for example, more accurate monitoring of changes in fitness potential of the connective tissue of interest.

According to the invention there is also provided a method for determining the fitness potential of a connective tissue of interest in a subject, the method comprising:
i) determining an echogenicity value of the connective tissue of interest in a first limb of the subject;
ii) determining an echogenicity value of an equivalent connective tissue in a second limb of the subject;
iii) determining a ratio of the echogenicity value of the connective tissue of interest in the first limb to the echogenicity value of the equivalent connective tissue in the second limb; and
iv) if the ratio determined in step (iii) is:
   a) less than one, determining that the fitness potential of the connective tissue of interest in the first limb is lower than the fitness potential of the equivalent connective tissue in the second limb; or
   b) greater than one, determining that the fitness potential of the connective tissue of interest in the first limb is higher than the fitness potential of the equivalent connective tissue in the second limb; or
   c) one, determining that the fitness potential of the connective tissue of interest in the first limb is the same as the fitness potential of the equivalent connective tissue in the second limb.

A method of the invention may be implemented on a general purpose computer, such as a laptop computer, desktop computer, tablet computer, or smart phone as a computerized method. The computerized method may be implemented on a general purpose computer in software such as an application or app.

Optionally the connective tissue in the second limb is healthy, and if the ratio determined in step (iii) is:
a) less than one, determining that the connective tissue of interest is injured or at risk of injury; or
b) greater than one, determining that the connective tissue of interest has a high fitness potential; or
c) one, determining that the connective tissue of interest is healthy.

Optionally steps (i) to (iii) are repeated after a period to determine whether there is a change in the fitness potential of the connective tissue of interest.

Optionally the period is at least one day, at least a week, at least two weeks, or at least a month.

There is further provided according to the invention a method for determining a change in the fitness potential of a connective tissue of interest in a subject, the method comprising:
i) determining an echogenicity value of a connective tissue of interest in a first limb of the subject;
ii) determining an echogenicity value of an equivalent connective tissue in a second limb of the subject;
iii) determining a ratio of the echogenicity value of the connective tissue of interest in the first limb to the echogenicity value of the connective tissue in the second limb;
iv) repeating steps (i) to (iii) after a period; and
v) if the ratio obtained in step (iv) is:
a) higher than the ratio obtained in step (iii), determining that the fitness potential of the connective tissue of interest has increased; or
b) the same as the ratio obtained in step (iii), determining that the fitness potential of the connective tissue of interest has remained the same; or
c) lower than the ratio obtained in step (iii), determining that the fitness potential of the connective tissue of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

Optionally the connective tissue of interest is an injured connective tissue, and if the ratio obtained in step (iv) is higher than the ratio obtained in step (iii), it is determined that the injury to the connective tissue of interest is healing.

Optionally the connective tissue of interest is a healthy connective tissue, and if the ratio obtained in step (iv) is lower than the ratio obtained in step (iii), it is determined that the connective tissue of interest is at risk of injury.

Optionally the second limb is a different limb of the same subject.

A connective tissue of interest may be in an upper limb or a forelimb of the subject, or in a lower limb or a hindlimb of the subject. A connective tissue of interest may, for example, be in a shoulder, arm, upper arm, lower arm, elbow, wrist, hand, or finger, a hip, leg, upper leg, lower leg, knee, ankle, foot, or toe of the subject.

An equivalent connective tissue may be in the opposite limb to the connective tissue of interest. For example, in a human, the connective tissue of interest may be in a finger of one hand (such as an index finger), and an equivalent connective tissue may be in the corresponding finger (such as an index finger) of the opposite hand. Similarly, the connective tissue of interest may be in a toe of one foot, and an equivalent connective tissue may be in a corresponding toe of the opposite foot. In a further example, the connective tissue of interest may be a tendon in an arm of the subject, and the equivalent connective tissue may be the corresponding tendon in the opposite arm of the subject. In specific examples in the foot, the connective tissue of interest may be the tibialias posterior tendon, Achilles Tendon or peroneal tendon, in humans. The equivalent tendon may, for example, be the corresponding tendon in the opposite foot.

The equivalent connective tissue may perform a similar function as the connective tissue of interest. For example, the connective tissue of intertest may be a flexor tendon, and the equivalent connective tissue may also be a flexor tendon. Alternatively, the connective tissue of intertest may be an extensor tendon, and the equivalent connective tissue may also be an extensor tendon. For example, the connective tissue of interest may be a flexor tendon (for example, a flexor tendon of a finger), and the equivalent connective tissue may also be a flexor tendon (for example, a flexor tendon in the corresponding finger of the opposite hand), rather than an extensor tendon.

It will be appreciated that, in other embodiments, instead of determining a ratio of the echogenicity value of the connective tissue of interest in the first limb to the echogenicity value of the connective tissue in the second limb in step (ii), a ratio of the echogenicity value of the connective tissue in the second limb to the echogenicity value of the connective tissue of interest in the first limb can instead be determined.

According to the invention there is also provided a method for determining the fitness potential of a connective tissue of interest in a subject, the method comprising:
i) determining an echogenicity value of the connective tissue of interest in a first limb of the subject;
ii) determining an echogenicity value of an equivalent connective tissue in a second limb of the subject;
iii) determining a ratio of the echogenicity value of the connective tissue in the second limb to the echogenicity value of the equivalent connective tissue of interest in the first limb; and
iv) if the ratio determined in step (iii) is:
   a) greater than one, determining that the fitness potential of the connective tissue of interest in the first limb is lower than the fitness potential of the equivalent connective tissue in the second limb; or
   b) less than one, determining that the fitness potential of the connective tissue of interest in the first limb is higher than the fitness potential of the equivalent connective tissue in the second limb; or
   c) one, determining that the fitness potential of the connective tissue of interest in the first limb is the same as the fitness potential of the equivalent connective tissue in the second limb.

A method of the invention may be implemented on a general purpose computer, such as a laptop computer, desktop computer, tablet computer, or smart phone as a computerized method. The computerized method may be implemented on a general purpose computer in software such as an application or app.

Optionally the connective tissue in the second limb is healthy, and if the ratio determined in step (iii) is:
a) greater than one, determining that the connective tissue of interest is injured or at risk of injury; or
b) less than one, determining that the connective tissue of interest has a high fitness potential; or
c) one, determining that the connective tissue of interest is healthy.

Optionally steps (i) to (iii) are repeated after a period to determine whether there is a change in the fitness potential of the connective tissue of interest.

Optionally the period is at least one day, at least a week, at least two weeks, or at least a month.

There is further provided according to the invention a method for determining a change in the fitness potential of a connective tissue of interest in a subject, the method comprising:
i) determining an echogenicity value of a connective tissue of interest in a first limb of the subject;
ii) determining an echogenicity value of an equivalent connective tissue in a second limb of the subject;
iii) determining a ratio of the echogenicity value of the connective tissue in the second limb to the echogenicity value of the connective tissue of interest in the first limb;
iv) repeating steps (i) to (iii) after a period; and
v) if the ratio obtained in step (iv) is:
a) lower than the ratio obtained in step (iii), determining that the fitness potential of the connective tissue of interest has increased; or
b) the same as the ratio obtained in step (iii), determining that the fitness potential of the connective tissue of interest has remained the same; or
c) higher than the ratio obtained in step (iii), determining that the fitness potential of the connective tissue of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

In such embodiments, optionally the connective tissue of interest is an injured connective tissue, and if the ratio obtained in step (iv) is lower than the ratio obtained in step (iii), it is determined that the injury to the connective tissue of interest is healing.

In such embodiments, optionally the connective tissue of interest is a healthy connective tissue, and if the ratio obtained in step (iv) is higher than the ratio obtained in step (iii), it is determined that the connective tissue of interest is at risk of injury.

Optionally the echogenicity value of the equivalent connective tissue in the second limb determined in step (ii) is a predetermined echogenicity value previously obtained for the connective tissue of the compared limb.

Optionally the predetermined echogenicity value was obtained when the connective tissue of the compared limb was healthy.

Optionally the echogenicity value of the connective tissue of interest obtained in step (i) is obtained after an injury to the connective tissue of interest.

Optionally the echogenicity value of the connective tissue of interest obtained in step (i) is obtained while the injury to the connective tissue of interest is healing.

Optionally the subject is a non-human subject.

Optionally the connective tissue of interest is in a forelimb, or an upper limb, of the subject, and the equivalent connective tissue is in the opposite forelimb, or opposite upper limb, of the subject.

Optionally the connective tissue of interest is in a hindlimb, or a lower limb, of the subject, and the equivalent connective tissue is in the opposite hindlimb, or opposite lower limb, of the subject.

Optionally the subject is a human subject.

Optionally the connective tissue of interest is in an upper limb of the subject, and the equivalent connective tissue is in the opposite upper limb of the subject.

Optionally the connective tissue of interest is in a lower limb of the subject, and the equivalent connective tissue is in the opposite lower limb of the subject.

Optionally the connective tissue is tendon or ligament tissue.

Optionally the connective tissue is a flexor tendon or an extensor tendon.

Optionally the first connective tissue is a tendon in a finger in a hand of the subject, and the second connective tissue is a tendon in a corresponding finger in the opposite hand of the subject.

Optionally the first connective tissue is a tendon in a toe in a foot of the subject, and the second connective tissue is a tendon in a corresponding toe in the opposite foot of the subject.

Optionally the first connective tissue comprises a tendon in a foot of the subject, wherein the tendon is selected from a tibial tendon, an Achilles tendon, or a peroneal tendon, and the second connective tissue comprises a corresponding tendon in the opposite foot of the subject.

Optionally the echogenicity values are determined quantitatively.

Optionally the echogenicity values are determined by ultrasonography.

Optionally the echogenicity values are determined by quantifying the echogenicity of ultrasonographic images.

Optionally the echogenicity is quantified by grey scale analysis of ultrasonographic images.

Optionally quantification by grey scale analysis of ultrasonographic images includes analysis of cross-sectional ultrasonographic images taken of the connective tissues.

Optionally quantification by grey scale analysis of ultrasonographic images includes analysis of longitudinal ultrasonographic images taken of the connective tissues.

Optionally a mean echogenicity value is determined for the connective tissue in the first limb, and a mean echogenicity value is determined for the connective tissue in the second limb.

Optionally the connective tissue of interest comprises damaged tissue.

Optionally a mean echogenicity value is determined for a lesion in the damaged tissue.

Optionally a mean echogenicity value is determined for tissue excluding a lesion in the damaged tissue.

Optionally the tissue damage is the result of an injury to the subject.

Optionally the injury is an acute injury.

Optionally the injury is a traumatic injury.

Optionally the injury is an overload injury.

Optionally the injury is a chronic injury.

Optionally the chronic injury is caused by long-term micro-trauma or degradation of the connective tissue.

Optionally the chronic injury is a caused by chronic inflammation about the tissue.

Optionally the first connective tissue comprises tendon tissue.

Optionally the second connective tissue comprises ligament tissue.

Optionally the first connective tissue comprises tendon tissue, and the second connective tissue comprises ligament tissue.

Optionally the first connective tissue comprises ligament tissue.

Optionally the second connective tissue comprises tendon tissue.

Optionally the first connective tissue comprises ligament tissue, and the second connective tissue comprises tendon tissue.

According to the invention there is also provided a method for determining the fitness potential of an area of interest in a tissue of a subject, the method comprising:
i) determining an echogenicity value of a first area of the tissue, and an echogenicity value of a second area of the same tissue spatially distant from the first area, wherein the area of interest is the first area;
ii) determining a relative difference of the echogenicity value of the first area to the echogenicity value of the second area; and
iii) if the relative difference determined in step (ii) is:
   a) less than one, determining that the fitness potential of the area of interest is lower than the fitness potential of the second area;
   b) greater than one, determining that the fitness potential of the area of interest is higher than the fitness potential of the second area; or
   c) one, determining that the fitness potential of the area of interest is the same as the fitness potential of the second area.

A method of the invention may be implemented on a general purpose computer, such as a laptop computer, desktop computer, tablet computer, or smart phone as a computerized method. The computerized method may be implemented on a general purpose computer in software such as an application or app.

Optionally the second area is healthy tissue, and in step (iii) the method comprises, if the relative difference determined in step (ii) is:
a) less than one, determining that the area of interest is injured or at risk of injury; or
b) greater than one, determining that the area of interest has a high fitness potential; or
c) one, determining that the area of interest is healthy.

Optionally the method further comprises repeating steps (i) to (iii) after a period to determine whether there is a change in the fitness potential of the area of interest.

Optionally the period is at least one day, at least a week, at least two weeks, or at least a month.

There is also provided according to the invention a method for determining a change in the fitness potential of an area of interest in tissue in a subject, the method comprising:
i) determining an echogenicity value of a first area of the tissue, and an echogenicity value of a second area of the same tissue spatially distant from the first area, wherein the area of interest is the first area of the tissue;
ii) determining a relative difference of the echogenicity value of the first area to the echogenicity value of the second area;
iii) repeating steps (i) and (ii) after a period; and
iv) if the relative difference obtained in step (iii) is:
a) higher than the relative difference obtained in step (ii), determining that the fitness potential of the area of interest has increased; or
b) similar to the relative difference obtained in step (ii), determining that the fitness potential of the area of interest has remained the same; or
c) lower than the relative difference obtained in step (ii), determining that the fitness potential of the area of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

Optionally the period is at least one day, at least a week, at least two weeks, or at least a month.

Optionally the area of interest comprises injured tissue, and if the relative difference obtained in step (iii) is higher than the relative difference obtained in step (ii), it is determined that the injury to the area of interest is healing.

Optionally the area of interest is healthy tissue, and if the relative difference obtained in step (iii) is lower than the relative difference obtained in step (ii), it is determined that the area of interest is at risk of injury.

Optionally a method of the invention further comprises:
a) comparing the relative difference obtained in step (ii) with a relative difference obtained for a corresponding first and second area for tissue of the same type as for the relative difference obtained in step (ii) but at the same or a different site for the same subject, or at the same or a different site for a different subject; and
b) if the relative difference obtained in step (ii) is:
   lower than the relative difference obtained in step (a), determining that the fitness potential of the area of interest is lower than the fitness potential of the corresponding first area of tissue in the compared tissue; or
   similar to the relative difference obtained in step (a), determining that the fitness potential of the area of interest is similar to the fitness potential of the corresponding first area of tissue in the compared tissue; or
   higher than the relative difference obtained in step (a), determining that the fitness potential of the area of interest is higher than the fitness potential of the corresponding first area of tissue in the compared tissue.

Optionally the corresponding first and second area in step (a) is at the same site for a different subject.

Optionally the corresponding first and second area in step (a) is at a different site for a different subject.

Optionally the corresponding first and second area in step (a) is at a same site for the same subject.

Optionally the corresponding first and second area in step (a) is at a different site for the same subject.

Optionally the relative difference obtained for the corresponding first and second area of the compared tissue in step (a) is a predetermined relative difference previously obtained for the first and second area of the compared tissue.

Optionally the predetermined relative difference was obtained when the area of interest was healthy.

Optionally the echogenicity value of the area of interest obtained in step (i) is obtained after an injury to the area of interest.

Optionally the predetermined relative difference was obtained after an injury to the area of interest.

Optionally the echogenicity value of the area of interest obtained in step (i) is obtained while the injury to the area of interest is healing.

Optionally the second area of tissue in step (i) is healthy tissue, and the first and second area of tissue in step (a) is healthy tissue, and wherein if the relative difference obtained in step (ii) is:
a) lower than the compared relative difference in step (a), determining that the area of interest is injured or at risk of injury; or
b) similar to the compared relative difference in step (a), determining that the area of interest is healthy; or
c) higher than the compared relative difference in step (a), determining that the area of interest has high fitness potential.

Optionally a method of the invention further comprises repeating steps (i) to (iii) after a period to determine whether there is a change in the fitness potential of the area of interest.

Optionally the period is at least one day, at least a week, at least two weeks, or at least a month.

There is also provided according to the invention a method for determining a change in the fitness potential of an area of interest in a tissue of a subject, the method comprising:
i) determining an echogenicity value of a first area of the tissue, and an echogenicity value of a second area of the same tissue spatially distant from the first area, wherein the area of interest is the first area;
ii) determining a relative difference between the echogenicity values obtained for the first and second areas in step (i);
iii) comparing the relative difference obtained in step (ii) with a relative difference obtained for a corresponding first and second area of the same tissue of the same or different subject;
iv) repeating steps (i) to (iii) after a period; and
v) determining whether there is a difference between the compared relative difference obtained in step (iii) and the compared relative difference obtained in step (iv); and if the compared relative difference obtained in step (iii) is:
   a) higher than the compared relative difference obtained in step (iv), determining that the fitness potential of the area of interest has improved; or
   b) similar to the compared relative difference obtained in step (iv), determining that the fitness potential of the area of interest has remained the same; or
   c) lower than the compared relative difference obtained in step (iv), determining that the fitness potential of the area of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

Optionally the period is at least one day, at least a week, at least two weeks, or at least a month.

Optionally the second area in step (i) is healthy tissue, and the first and second areas in step (iii) are healthy connective tissues.

Optionally the area of interest comprises injured tissue, and if the compared relative difference obtained in step (iii) is higher than the compared relative difference obtained in step (iv), determining that the injury to the area of interest is healing.

Optionally the area of interest is healthy tissue, and if the compared relative difference obtained in step (iii) is lower than the compared relative difference obtained in step (iv), determining that that the area of interest is at risk of injury.

Optionally the tissue comprises connective tissue.

Optionally the tissue comprises tendon or ligament tissue.

Optionally the tissue comprises muscle tissue.

Optionally the tissue comprises cardiac muscle tissue.

There is also provided according to the invention a method for determining the fitness potential of a muscle tissue of interest, wherein the method comprises:
i) determining an echogenicity value of a muscle tissue of interest; and
ii) if the echogenicity value obtained in (i) is:
   a) less than an echogenicity value for a corresponding muscle tissue, determining that the fitness potential of the tissue of interest is lower than the fitness potential of the corresponding muscle tissue;
   b) greater than an echogenicity value for the corresponding muscle tissue, determining that the fitness potential of the tissue of interest is higher than the fitness potential of the corresponding muscle tissue; or
   c) the same as an echogenicity value for the corresponding muscle tissue, determining that the fitness potential of the tissue of interest is the same as the fitness potential of the corresponding muscle tissue.

A method of the invention may be implemented on a general purpose computer, such as a laptop computer, desktop computer, tablet computer, or smart phone as a computerized method. The computerized method may be implemented on a general purpose computer in software such as an application or app.

Optionally the corresponding muscle tissue is healthy tissue, and in step (ii) the method comprises, if the echogenicity value determined in step (i) is:
a) less than an echogenicity value for the corresponding muscle tissue, determining that the tissue of interest is injured or at risk of injury;
b) greater than an echogenicity value for the corresponding muscle tissue, determining that the tissue of interest has high fitness potential; or
c) the same as an echogenicity value for the corresponding muscle tissue, determining that the tissue of interest is healthy.

Optionally the method further comprises repeating steps (i) and (ii) after a period to determine whether there is a change in the fitness potential of the tissue of interest.

Optionally the period is at least one day, at least a week, at least two weeks, or at least a month.

Optionally the corresponding muscle tissue is the same muscle tissue of the same subject.

Optionally the echogenicity value of the corresponding muscle tissue is a predetermined value previously obtained.

Optionally the echogenicity value of the corresponding muscle tissue is a predetermined value previously obtained from the same subject.

Optionally the predetermined echogenicity value was obtained when the muscle tissue of interest was healthy.

Optionally the echogenicity value of the muscle tissue of interest obtained in step (i) was obtained after an injury to the muscle tissue.

Optionally the corresponding muscle tissue is the same muscle tissue of a different subject.

Optionally the echogenicity value of the corresponding muscle tissue is a predetermined echogenicity value obtained from a different subject.

Optionally the echogenicity value of the corresponding muscle tissue was obtained after an injury to the muscle tissue of interest.

Optionally the echogenicity of the muscle tissue of interest obtained in step (i) is obtained while the injury to the muscle tissue of interest is healing.

There is also provided according to the invention a method for determining a change in the fitness potential of a muscle tissue of interest in a subject, the method comprising:
i) determining an echogenicity value of the muscle tissue of interest;
ii) repeating step (i) after a period; and
iii) if the value obtained in step (ii) is:
   a) higher than the value obtained in step (i), determining that the fitness potential of the muscle tissue of interest has increased; or
   b) similar to the value obtained in step (i), determining that the fitness potential of the muscle tissue of interest has remained the same; or
   c) lower than the value obtained in step (i), determining that the fitness potential of the muscle tissue of interest has reduced.

A method of the invention may be implemented on a general purpose computer, such as a laptop computer, desktop computer, tablet computer, or smart phone as a computerized method. The computerized method may be implemented on a general purpose computer in software such as an application or app.

Optionally the period is at least one day, at least a week, at least two weeks, or at least a month.

Optionally the muscle tissue of interest is an injured tissue, and if the value obtained in step (ii) is higher than the value obtained in step (i), it is determined that the injury to the tissue of interest is healing.

Optionally the muscle tissue of interest is a healthy tissue, and if the value obtained in step (iii) is lower than the value obtained in step (ii), it is determined that the tissue of interest is at risk of injury.

Optionally the muscle tissue is cardiac muscle tissue.

Optionally the echogenicity values are determined quantitatively.

Optionally the echogenicity values are determined by ultrasonography.

Optionally the echogenicity values are determined by quantifying the echogenicity of ultrasonographic images.

Optionally the echogenicity is quantified by grey scale analysis of ultrasonographic images.

Optionally a method of the invention includes analysis of cross-sectional ultrasonographic images taken of the tissues.

Optionally a method of the invention includes analysis of longitudinal ultrasonographic images taken of the tissues.

Optionally a mean echogenicity value is determined for each tissue.

Optionally the tissue of interest comprises damaged tissue.

Optionally a mean echogenicity value is determined for a lesion in the damaged tissue.

Optionally a mean echogenicity value is determined for tissue excluding a lesion in the damaged tissue.

Optionally the tissue damage is the result of an injury to the subject.

Optionally the injury is an acute injury.

Optionally the muscle tissue is cardiac muscle tissue, and the injury is caused by decreased or complete cessation of blood flow to the cardiac tissue.

Optionally the injury is caused by a full or partial occlusion of a blood vessel.

Optionally the blood vessel is a coronary artery.

Optionally the injury is caused by a tear or strain.

Optionally the muscle is cardiac muscle and the injury is caused by blunt cardiac injury.

Optionally the injury is a traumatic injury.

Optionally the injury is an overload injury.

Optionally the injury is a chronic injury.

Optionally the injury is caused by long-term disruption of blood flow to the tissue.

Optionally the tissue is cardiac tissue and the injury is caused by atherosclerosis.

Optionally the chronic injury is caused by long-term micro-trauma or degradation of the connective tissue.

Optionally the chronic injury is a caused by chronic inflammation about the tissue.

Optionally the muscle tissue is human muscle tissue.

Optionally the subject is a human subject.

Embodiments of the invention are described in the following examples, with reference to the accompanying drawings in which:

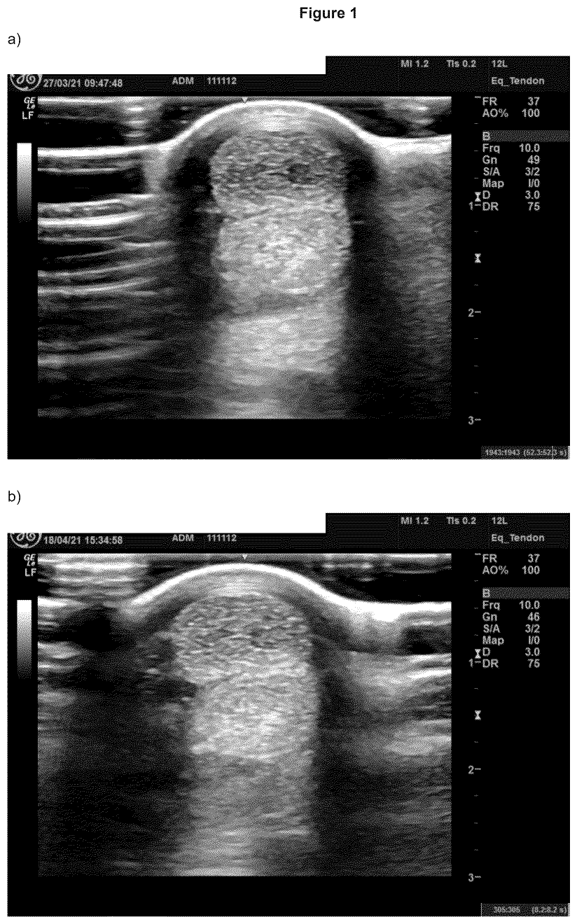

FIG. 1 shows an ultrasonogram displayed on a computer display of a subtle core lesion in the lateral aspect of superficial digital flexor tendon (SDFT) with generalised surrounding tendonitis of a horse (ID 111112): (a) before administration of any pharmaceutical composition comprising vitamin A; and (b) after daily administration of a pharmaceutical composition comprising vitamin A for 14 days;

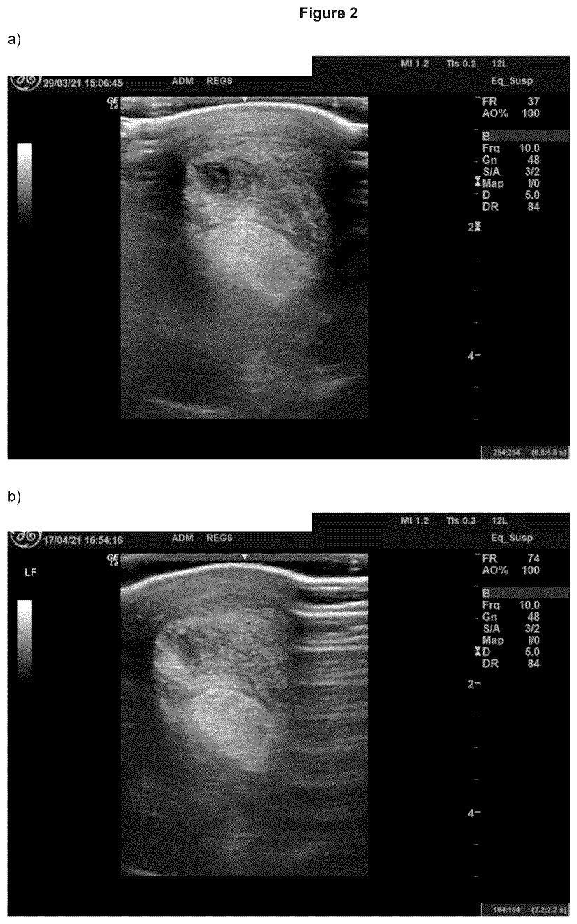

FIG. 2 shows an ultrasonogram displayed on a computer display of a nasty SDFT core lesion in the medial aspect not quite involving paratenon for a horse (ID REG6): (a) before administration of any pharmaceutical composition comprising vitamin A; and (b) after daily administration of a pharmaceutical composition comprising vitamin A for 14 days;

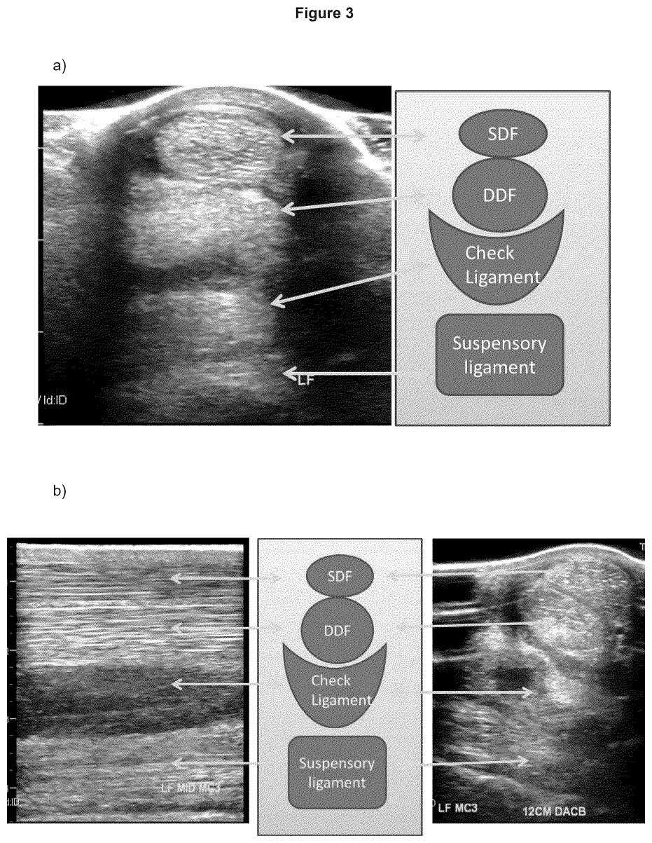

FIG. 3(a) shows a cross-sectional ultrasonogram displayed on a computer display of the major tendons/ligaments present in the left foot of a horse (normal equine anatomy). FIG. 3(b) shows a longitudinal ultrasonogram (left) of the left foot of a horse compared with a cross-sectional ultrasonogram (right) of the left foot of the same horse (normal equine anatomy);

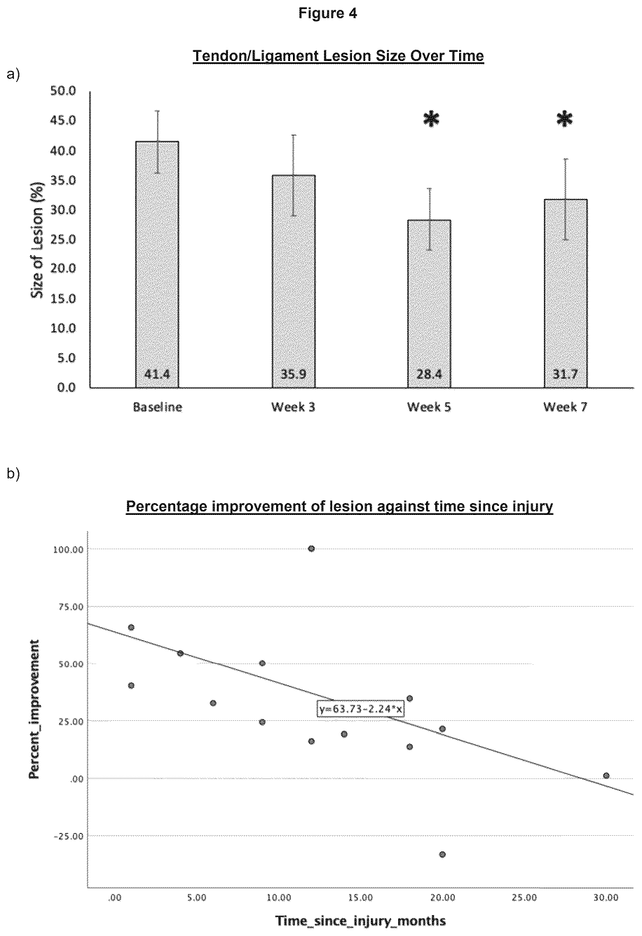

FIG. 4(a) shows a bar graph depicting size of lesion (as a % of baseline lesion size) in tendon/ligament at specified time points after commencing supplementation. Data are presented as mean values, with error bars representing the standard error of the mean. Statistically significant results (p<0.05) compared to baseline were noted with an asterisk (*). Week 3: p=0.169; Week 5: p=<0.001; Week 7: p=0.006. FIG. 4(b) shows scatter plots of percentage improvement (%) against time since injury (months) with all data points included (Pearson's correlation p=0.027);

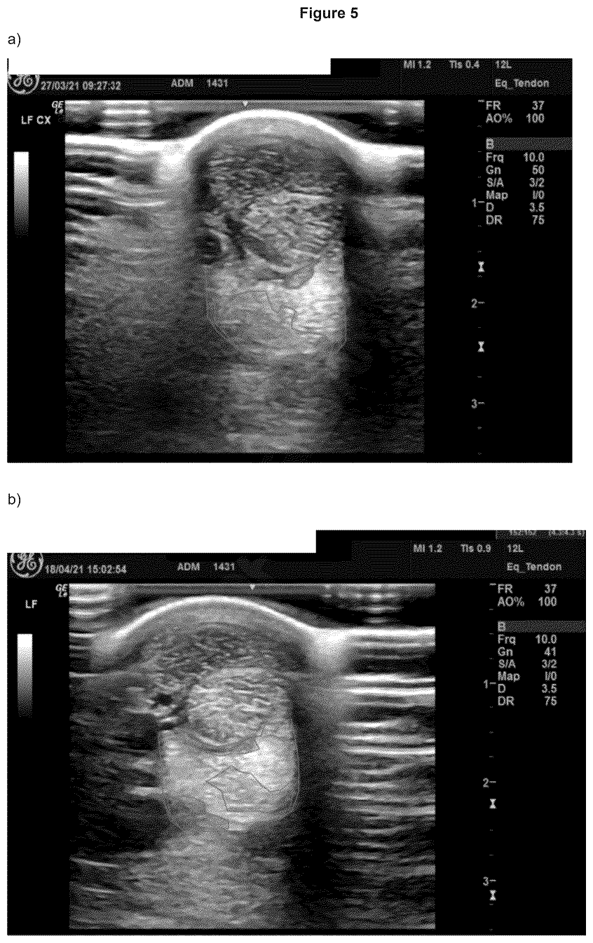

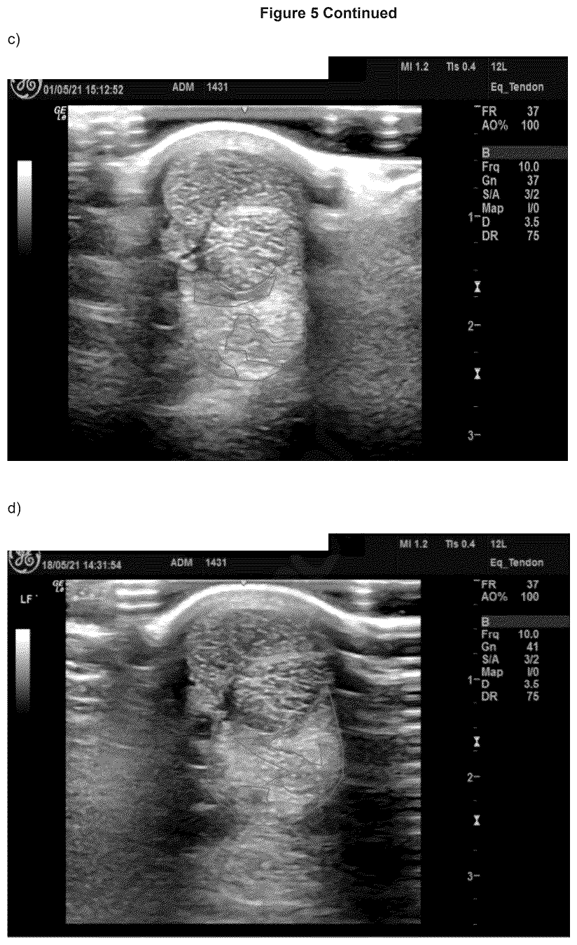

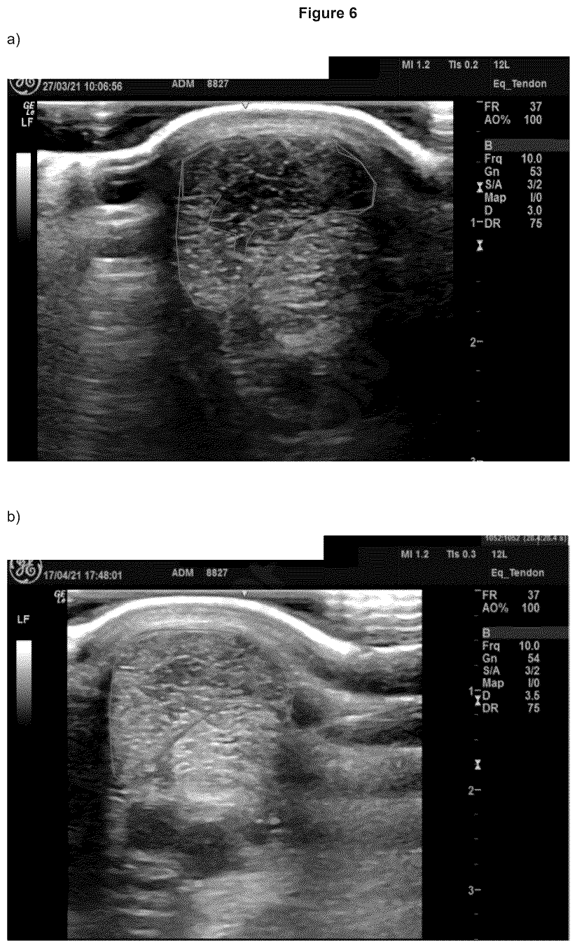

FIG. 5 shows a cross-sectional ultrasonogram displayed on a computer display of a lesion in the left forelimb check ligament in a horse (ID 1431): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. There is movement artefact shown in the figure;

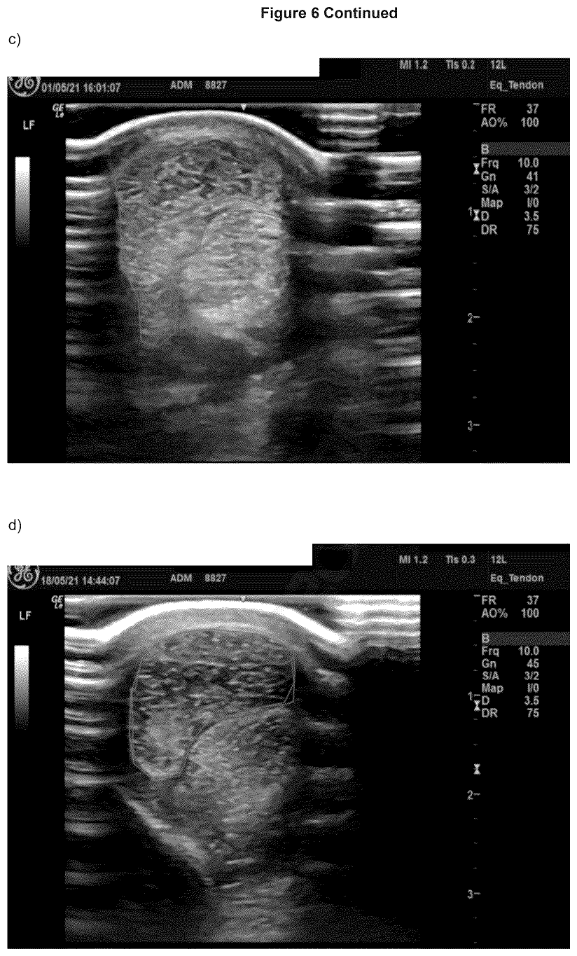

FIG. 6 shows a cross-sectional ultrasonogram displayed on a computer display of a lesion in the left forelimb SDFT in a horse (ID 8827): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks;

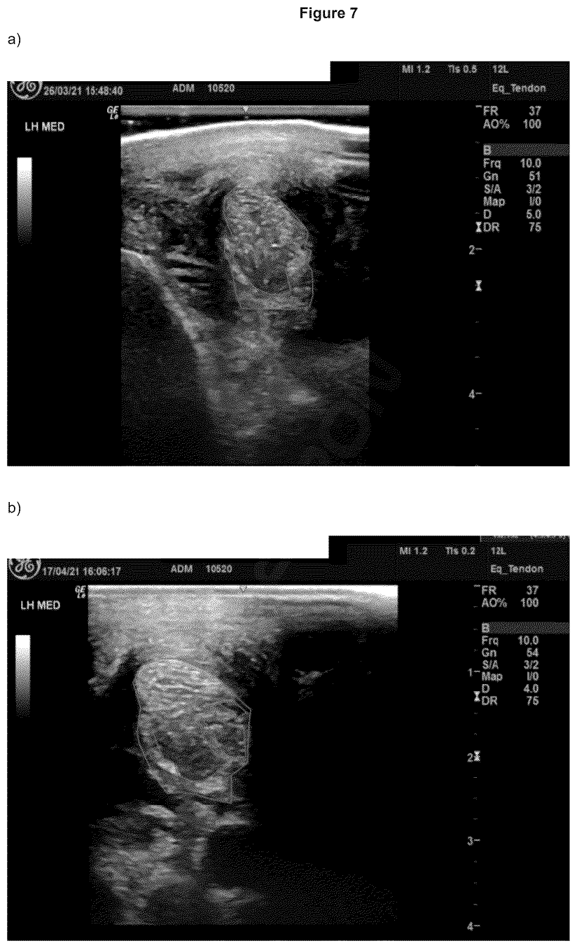

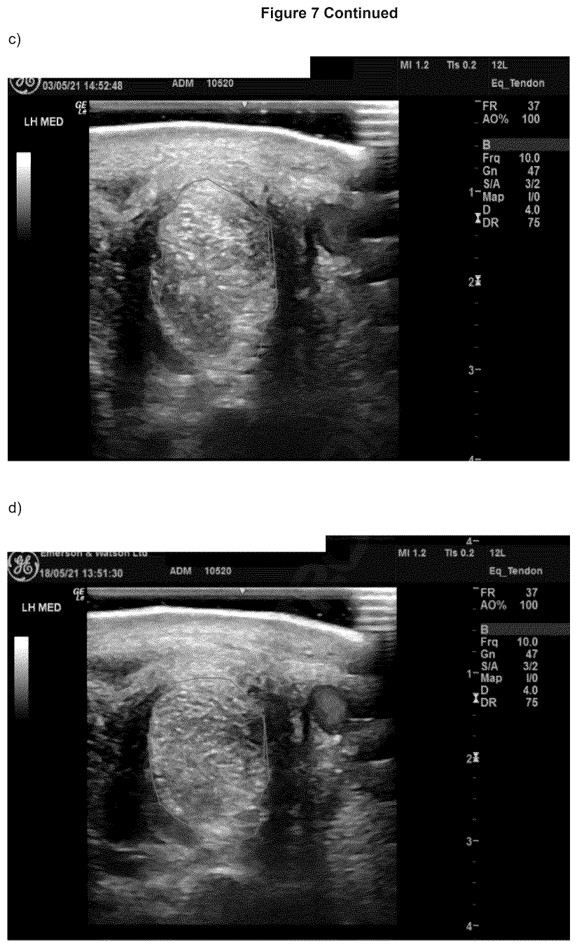

FIG. 7 shows a cross-sectional ultrasonogram displayed on a computer display of a lesion in the left hindlimb medial suspensory branch ligament in a horse (ID 10520): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. (d) shows improvements in axial aspect of the branch;

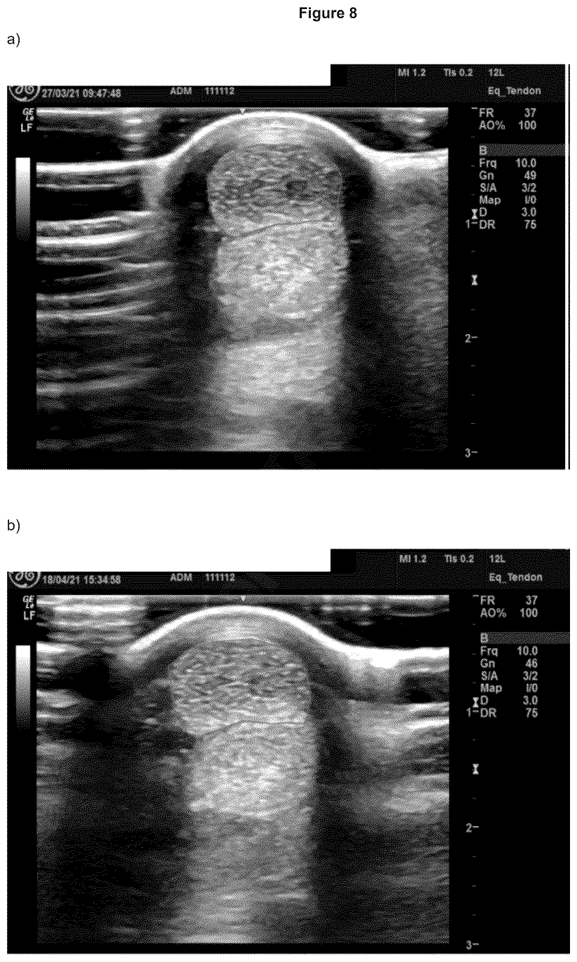

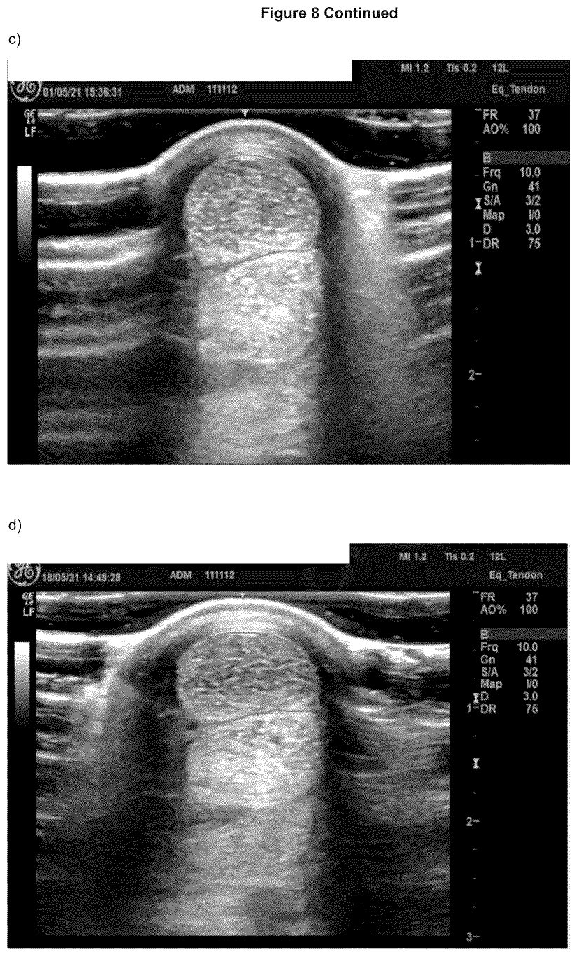

FIG. 8 shows a cross-sectional ultrasonogram displayed on a computer display of a lesion in the left forelimb SDFT in a horse (ID 111112): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. Some filling in of lesion in lateral aspect of tendon;

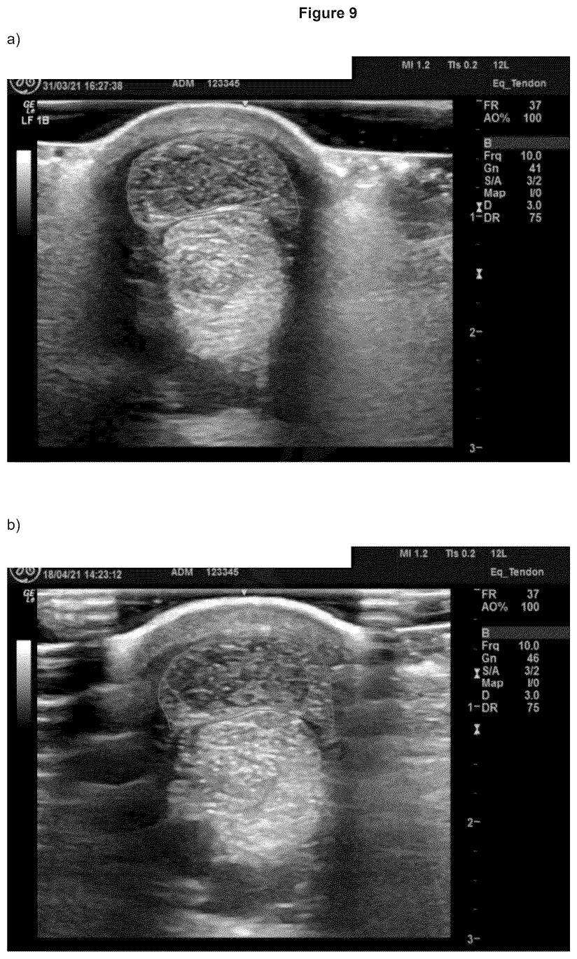

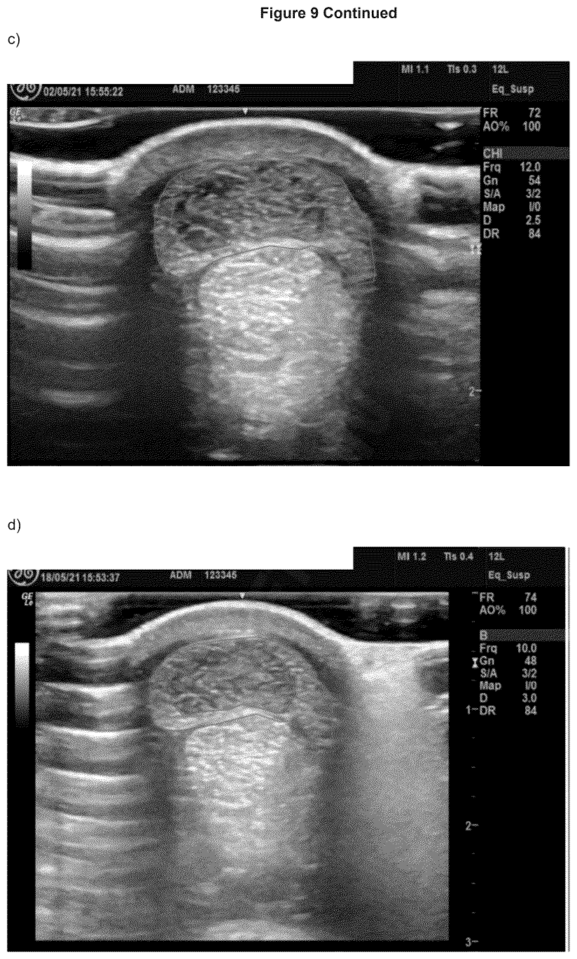

FIG. 9 shows a cross-sectional ultrasonogram displayed on a computer display of a lesion in the left forelimb SDFT in a horse (ID 123345): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks;

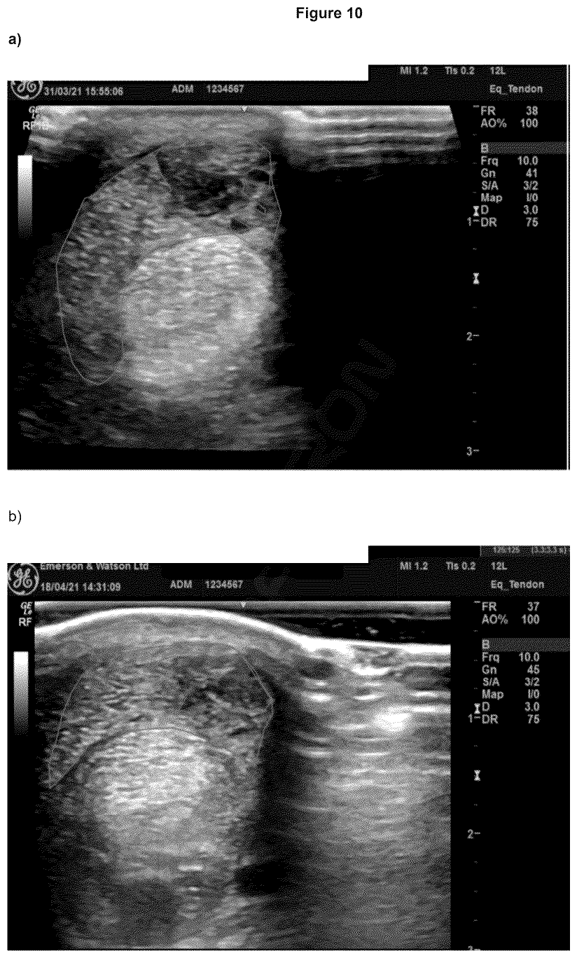

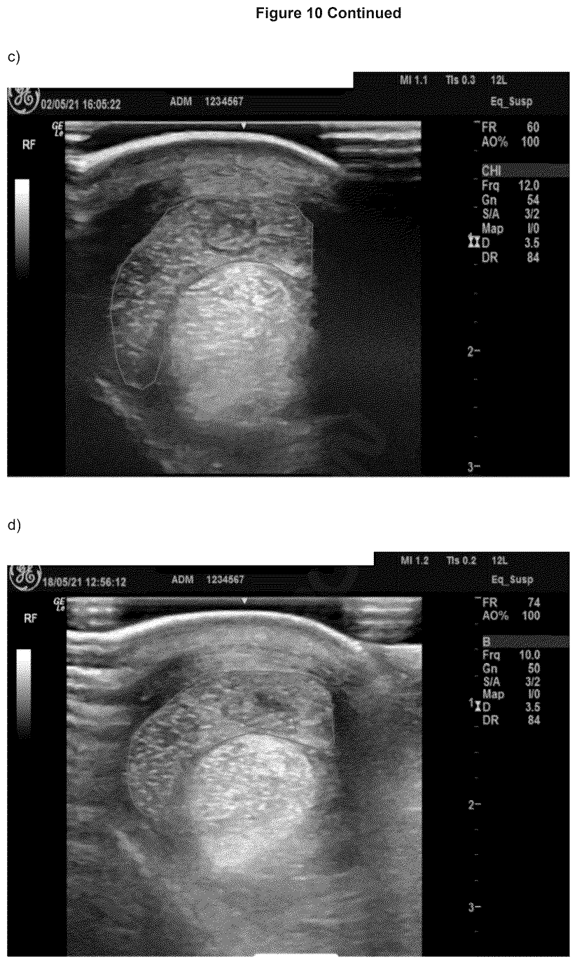

FIG. 10 shows a cross-sectional ultrasonogram displayed on a computer display of a lesion in the right forelimb lateral SDFT in a horse (ID 1234567): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. Tendon is clearly filling in well;

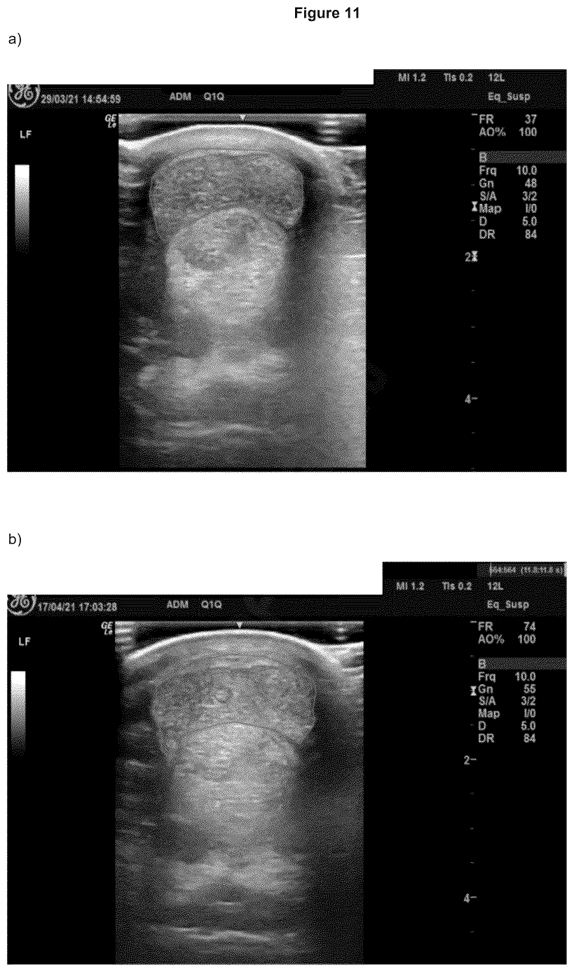

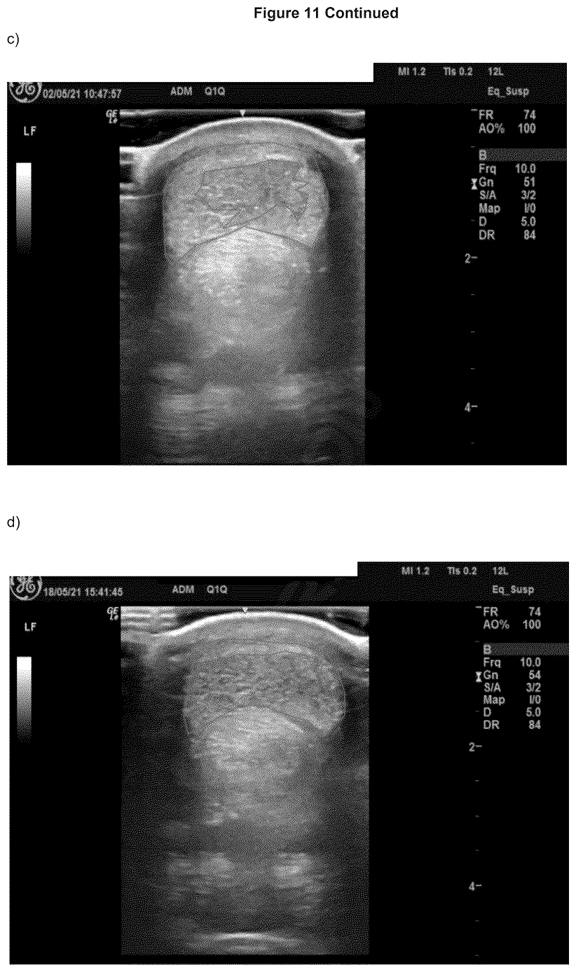

FIG. 11 shows a cross-sectional ultrasonogram displayed on a computer display of a lesion in the left forelimb SDFT in a horse (ID Q1Q): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks;

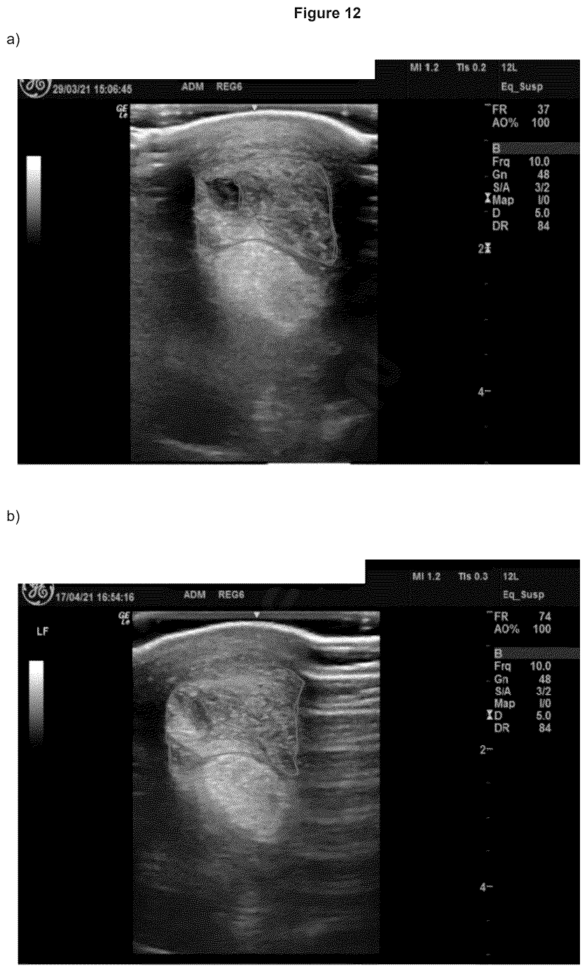

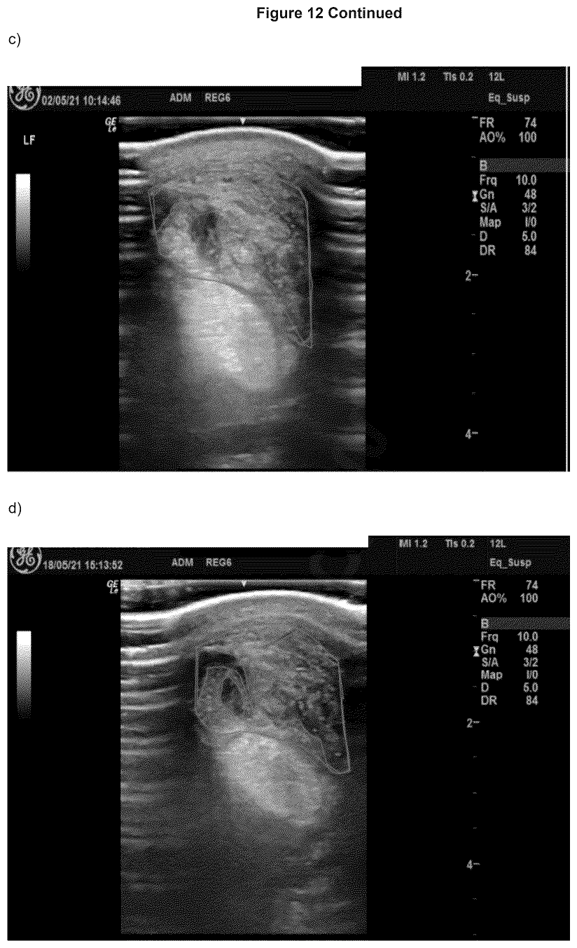

FIG. 12 shows a cross-sectional ultrasonogram displayed on a computer display of a lesion in the left forelimb SDFT in a horse (ID REG6): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks;

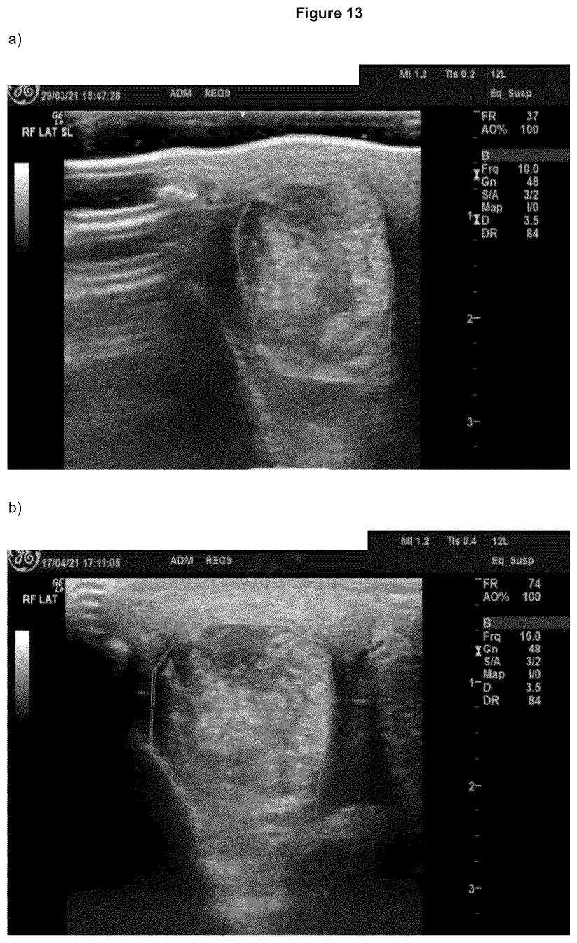

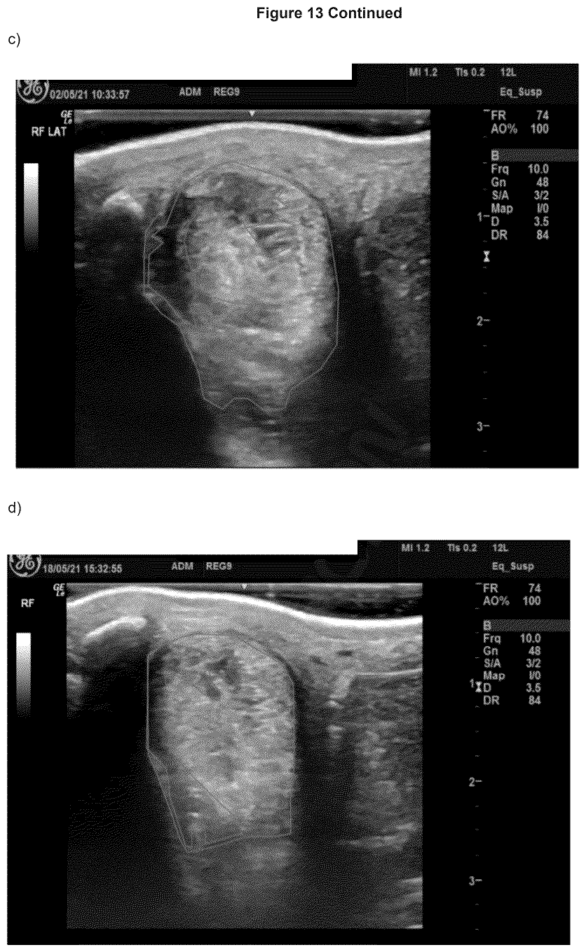

FIG. 13 shows a longitudinal ultrasonogram displayed on a computer display of a lesion in the right forelimb lateral suspensory branch ligament in a horse (ID REG9): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. Image at week 7 (FIG. 12(d)) shows improvement of injury as deeper lesion less visible;

FIG. 14 shows a cross-sectional ultrasonogram displayed on a computer display of a lesion in the left forelimb check ligament in a horse (ID 1833): (a) before administration of any vitamin A supplement comprising vitamin A; and (b) after daily administration of a pharmaceutical composition comprising vitamin A for 5 weeks. Some improvement of check ligament injury appearance. The horse also received platelet rich plasma;

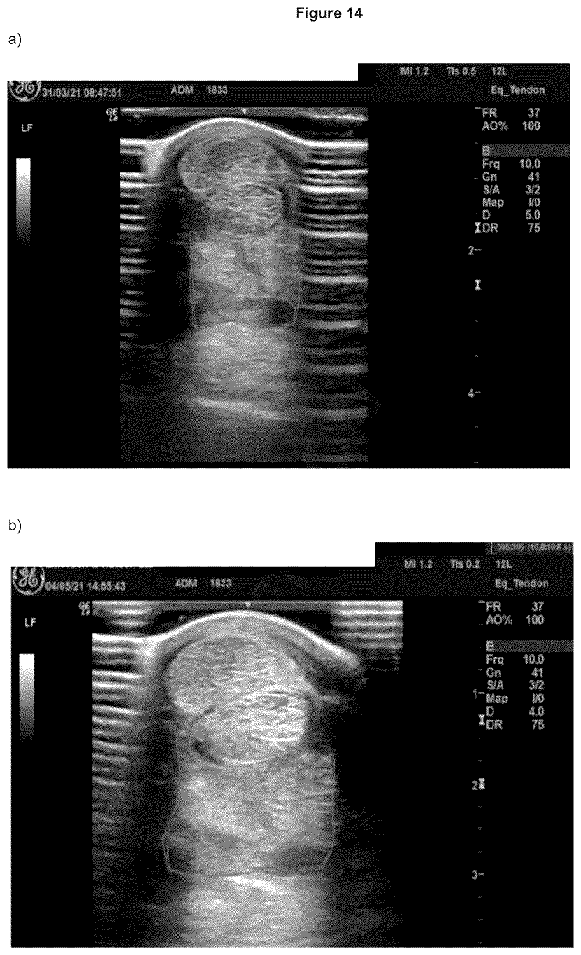

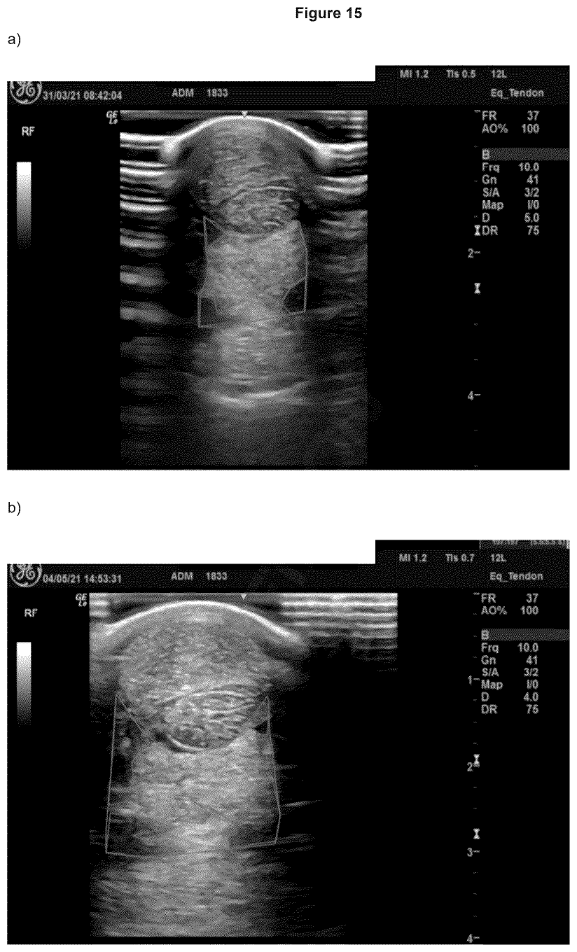

FIG. 15 shows a cross-sectional ultrasonogram displayed on a computer display of a lesion in the right forelimb check ligament in a horse (ID 1833): (a) before administration of any vitamin A supplement comprising vitamin A; and (b) after daily administration of a pharmaceutical composition comprising vitamin A for 5 weeks;

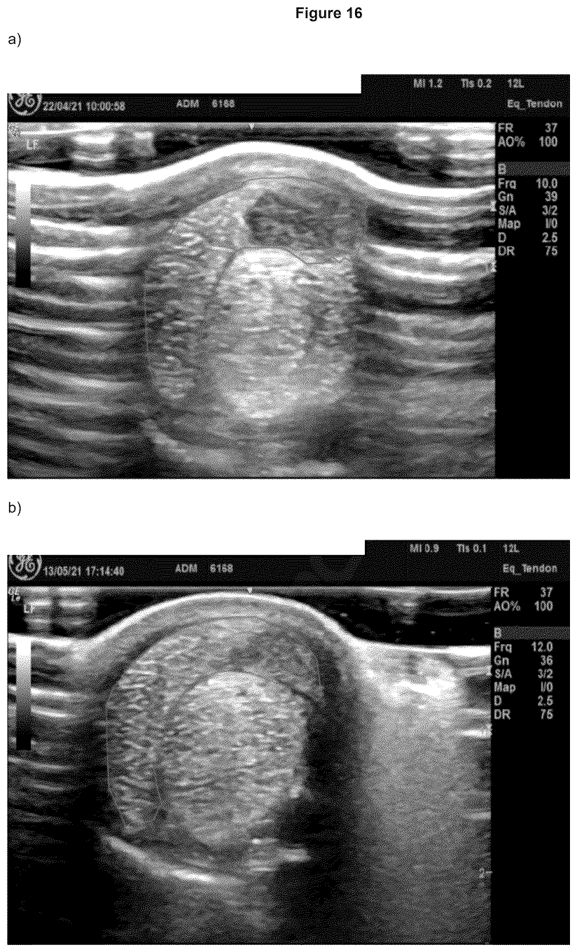

FIG. 16 shows a cross-sectional ultrasonogram displayed on a computer display of a lesion in the left forelimb lateral SDFT in a horse (ID 6168): (a) before administration of any vitamin A supplement comprising vitamin A; and (b) after daily administration of a pharmaceutical composition comprising vitamin A for 3 weeks. The figure shows good infilling of lateral SDFT lesion;

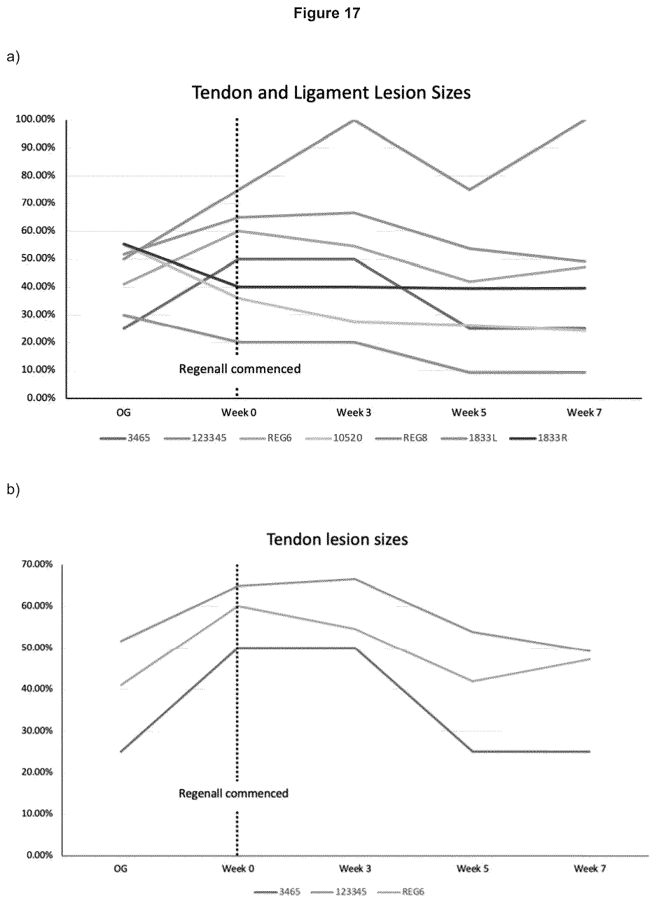

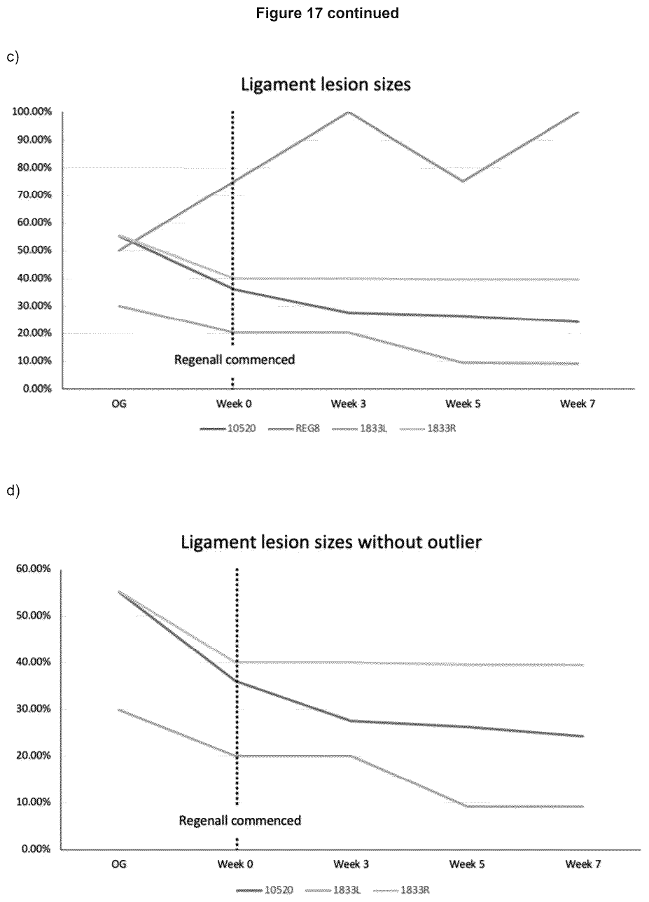

FIG. 17 shows line graphs depicting size of lesion (as a % of baseline lesion size) in tendon/ligament injuries at specified time points after original injury (OG): (a) shows tendon/ligaments as a single cohort; (b) shows tendon injuries as a separate, single cohort; (c) shows ligamentous injuries as a separate, single cohort; and (d) shows ligamentous injuries as a separate, single cohort with outlier removed from data set. Data are presented as actual values for each subject;

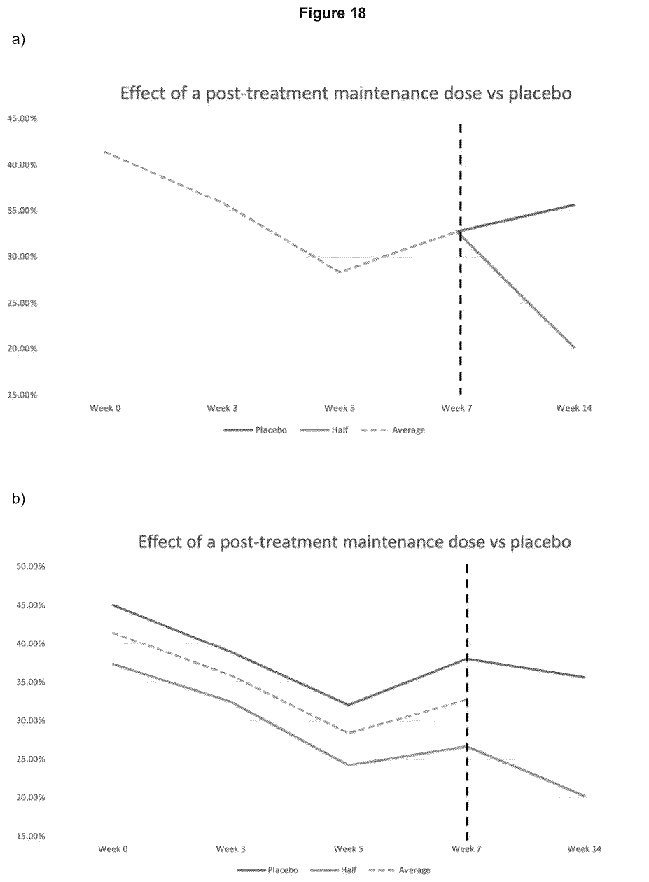

FIG. 18 shows a line graph depicting the effect of post-treatment maintenance dose of vitamin A supplement on the size of tendon/ligamentous lesions: (a) shows mean lesion size from week 0 to week 7 on full-treatment doses of vitamin A supplement before splitting into the values of the maintenance dose and placebo groups for week 7 to week 14; and (b) shows the lesion size for both maintenance and placebo groups from week 0 through to week 14, as well as the mean values for the groups from week 0 to week 7;

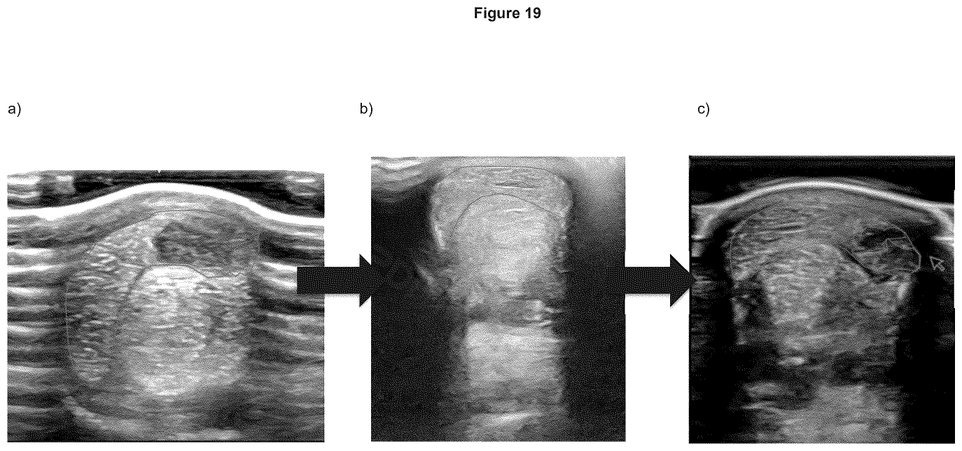

FIG. 19 shows a series of cross-sectional ultrasonograms each displayed on a computer display of a tendon injury that became re-injured after administration of full-treatment doses of vitamin A supplement was stopped and a maintenance dose of supplement was administered. Figure (a) shows an ultrasonogram of the lesion at baseline (week 0) before treatment with vitamin A supplement commenced; (b) shows an ultrasonogram at week 7 of administration with full-treatment doses of vitamin A supplement; and (c) shows ultrasonogram of the lesion at week 14 (after 7 weeks of post full-treatment maintenance doses of vitamin A supplement);

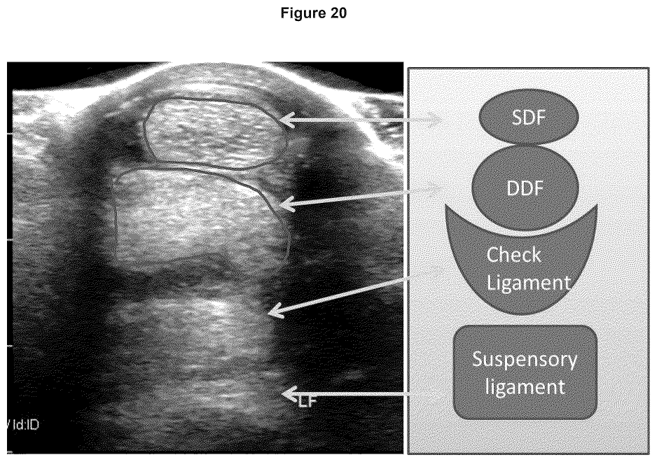

FIG. 20 shows a cross-sectional ultrasonogram displayed on a computer display of the major tendons/ligaments present in the left foot of a horse (with normal equine anatomy) displayed on a computer display. Outlines of the SDFT and adjacent DDFT are shown;

FIG. 21 shows a diagram of supportive tendons and ligaments of the equine foot, including the SDFT, DDFT, LDET, CDET, and check ligament;

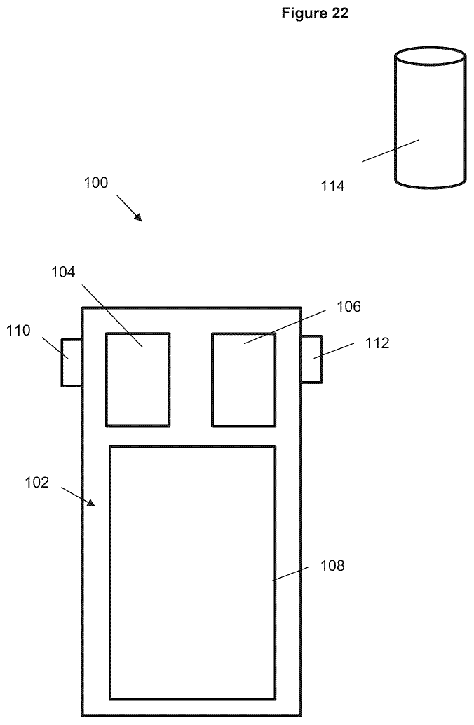

FIG. 22 is a schematic diagram of a computer system for implementing methods described herein;

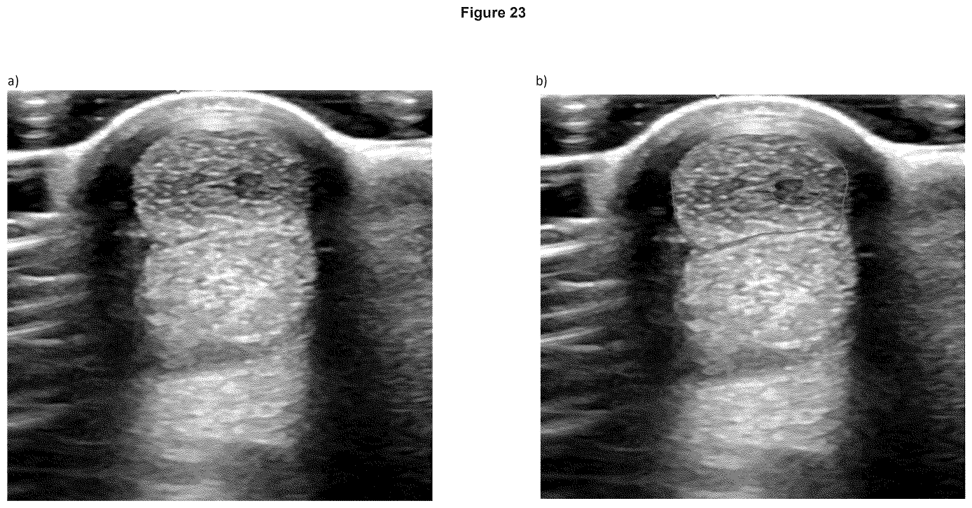

FIG. 23(a) shows a cross-sectional ultrasonogram of the major tendons/ligaments present in the foot of a horse, with a lesion present in the SDFT displayed on a computer display. FIG. 23(b) shows the outline of the SDFT, and the outline of the lesion within it displayed on a computer display;

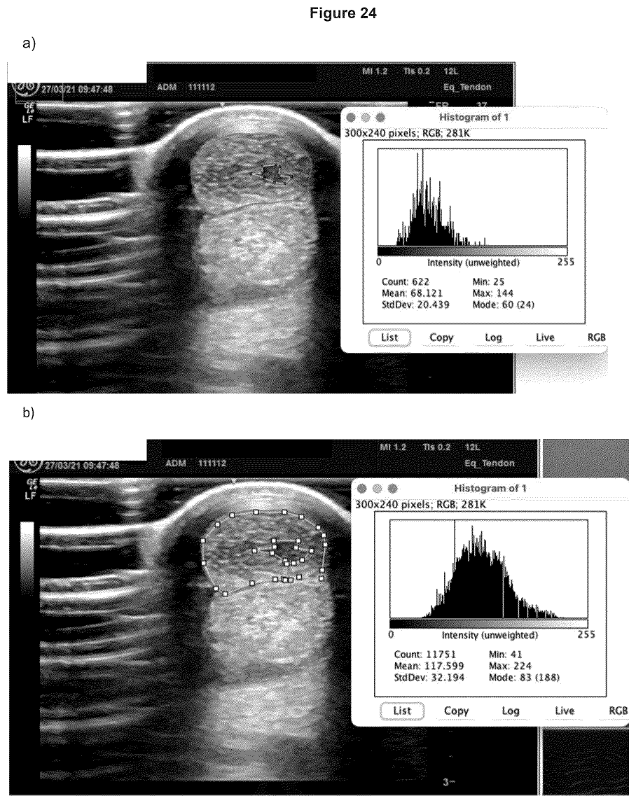

FIG. 24 shows cross-sectional ultrasonograms of the major tendons/ligaments present in the foot of a horse, with a lesion present in the SDFT displayed on a computer display. The outline of the SDFT, and the outline of the lesion within the SDFT are shown. In FIG. 24(a), a histogram shows the counts within the area of the lesion. In FIG. 24(b), a histogram shows the counts within the area of the SDFT excluding the lesion;

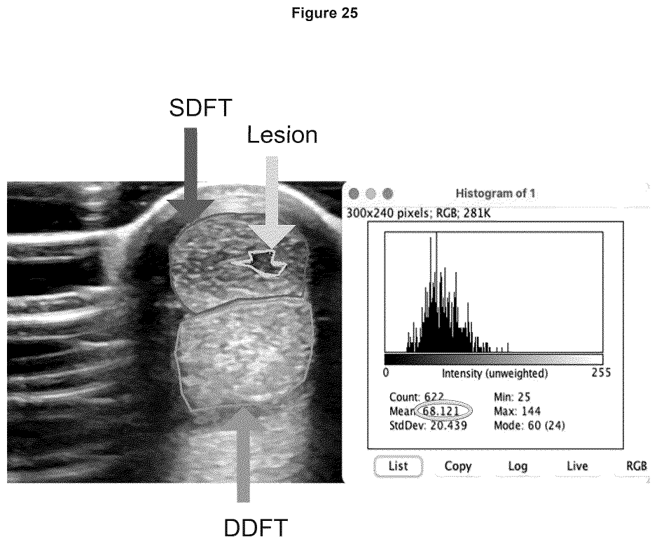

FIG. 25 shows a cross-sectional ultrasonogram of the SDFT and DDFT present in the foot of a horse, with a lesion present in the SDFT displayed on a computer display. The outline of the SDFT is shown in red, the outline of the lesion within the SDFT is shown in yellow, and the outline of the adjacent DDFT is shown in green. A histogram shows the counts within the area of the lesion;

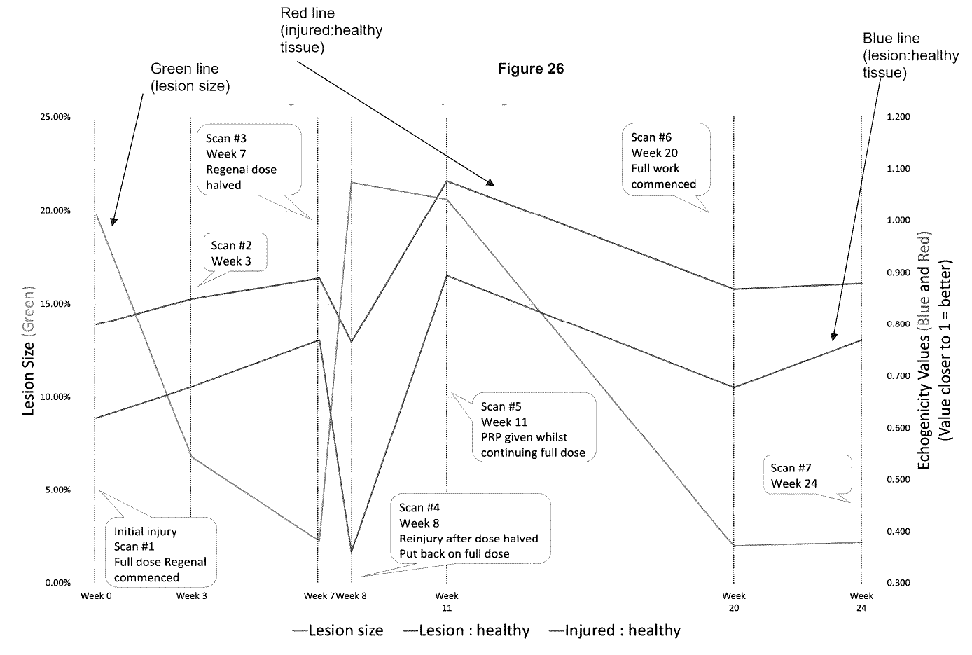

FIG. 26 shows the effect of administration of vitamin A and PRP on equine tendon injury. The figure shows a line graph depicting lesion size (as a percentage of baseline lesion size) and echogenicity values (blue line is ratio of echogenicity of lesion:healthy corresponding tendon, red line is ratio of injured tendon:healthy corresponding tissue) in an injured equine tendon at specified time points after initial injury. Full dose of vitamin A commenced at week 0, which was replaced with maintenance dose at week 7, at week 8 the horse was put back on full-dose vitamin A before commencing PRP treatment at week 11;

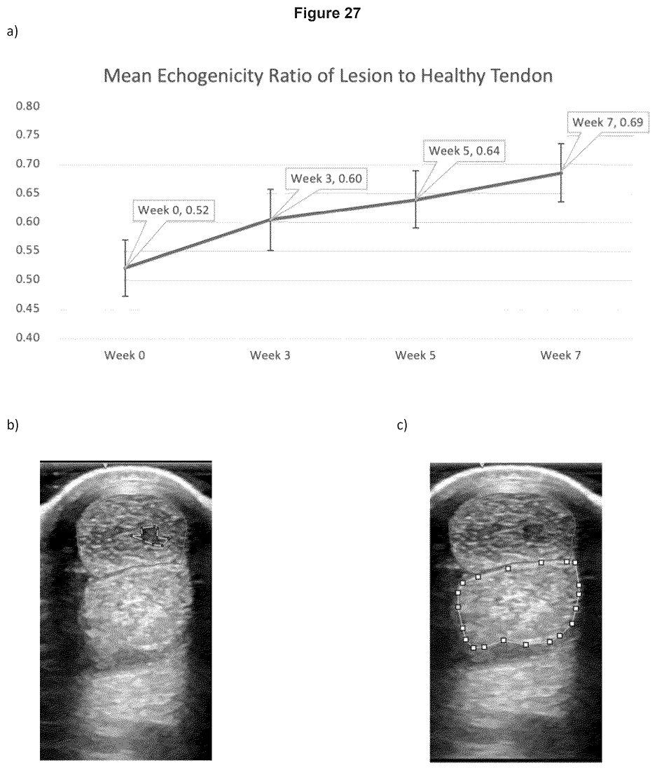

FIG. 27(a) shows a line graph depicting the mean echogenicity ratio of a tendon lesion to an adjacent healthy tendon at specified time points after commencing supplementation. Data are presented as mean values for the tissues, with error bars representing the standard error of the mean. A value of 1 would indicate perfect regeneration of native tendon. FIG. 27(b) shows a cross-sectional ultrasonogram displayed on a computer display showing the outline of the tendon lesion of (a) with the injured tendon also outlined. FIG. 27(c) shows a cross-sectional ultrasonogram displayed on a computer display showing the outline of an adjacent healthy tendon used as a comparison tissue to calculate the echogenicity ratio, as well as the outline of the lesion and injured tendon; and

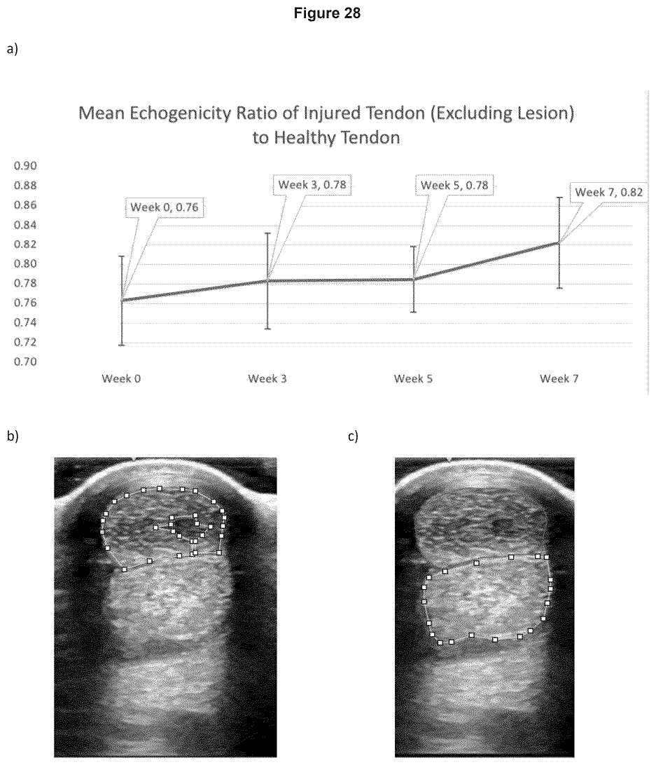

FIG. 28(a) shows a line graph depicting the mean echogenicity ratio of an injured tendon (with the area of lesion excluded) to an adjacent healthy tendon at specified time points after commencing supplementation. Data are presented as mean values for the tissues, with error bars representing the standard error of the mean. A value of 1 would indicate perfect regeneration of native tendon. FIG. 28(b) shows a cross-sectional ultrasonogram, displayed on a computer display, showing the outline of the tendon lesion of (a) with the injured tendon also outlined, the lesion is excluded from the area of injured tendon for analysis. FIG. 28(c) shows a cross-sectional ultrasonogram, displayed on a computer display, showing the outline of an adjacent healthy tendon used as a comparison tissue to calculate the echogenicity ratio, as well as the outline of the lesion and injured tendon.

### EXAMPLES

### Example 1—Treatment of Equine Tendon Injury

This example describes the effect of a pharmaceutical composition comprising vitamin A in treating tendon injury in horses.

Tendon injuries result in the formation of a fibrovascular scar that never attains the characteristics of normal tendon. Tendon healing is characterised by the formation of fibrovascular scar tissue, as tendon has very little intrinsic regenerative capacity. The molecular mechanisms resulting in scar tissue formation after tendon injuries are not well understood (as reviewed in Schneider et al. Rescue plan for Achilles: Therapeutics steering the fate and functions of stem cells in tendon wound healing; Advanced Drug Delivery Reviews 129 2018 352-375). Briefly, in the first few days after injury a blood clot forms that serves as a preliminary scaffold for invading cells followed by a more robust vascular network which is essential for the survival of tenocytes engaged in the synthesis of new fibrous tissue. Thereafter, fibroblasts are recruited to the injured site and produce initially disorganised extracellular matrix components. Following this, a remodelling stage commences characterised by tissue changes resulting in a more fibrous appearance and eventually a scar-like tendon tissue can be observed.

Current biologic treatment strategies have not achieved tendon regeneration but include the use of extracellular matrix patches to provide a scaffold for new cell growth and differentiation (as reviewed in Galatz et al. Tendon Regeneration and Scar Formation: The Concept of Scarless Healing, J. Orthop. Res. 2015, 33(6) 823-831). Platelet rich plasma which comprises a multitude of growth factors normally involved in repair processes has also been investigated for use in tendon repair. However, there is no evidence that either strategy induces tendon regeneration. Tendon injuries are also a particular problem in horses.

Tendon injury has a similar pathophysiology to injury in other tissues (including spinal cord injury) in that they may be characterised by excessive deposition of scar tissue. Evidence for an effective treatment of tendon injury (including evidence for inhibition of scar tissue formation following tendon injury) is considered to provide evidence also for an effective treatment of other types of injury (including spinal cord injury), for example, through inhibition of scar tissue formation.

### Pharmaceutical Composition Used:

Vitamin A palmitate (also known as preformed vitamin A, or retinyl palmitate) mixed with coconut oil to provide a final vitamin A concentration of 10,000 IU/ml.

### Administration of Pharmaceutical Composition:

Horses with tendon injury were orally administered vitamin A palmitate mixed with coconut oil, at a dose of 160,000 IU once per day for 14 days.

### Results

Ultrasonographs of the lesions before administration of any pharmaceutical composition comprising vitamin A, and after daily administration of the composition for 14 days are shown for two different horses in FIGS. 1 and 2 (FIG. 1: horse ID 111112; FIG. 2: horse ID REG6).

FIG. 1(a) (before any administration of the composition) shows a subtle core lesion in the lateral aspect of the superficial digital flexor tendon (SDFT) with generalised surrounding tendonitis. FIG. 1(b) (after daily administration of the composition for 14 days) shows that the core lesion has filled in somewhat and is less hypoechoic, suggesting that something has “plugged” the hole. Whilst the nature and quality of the tissue in the lesion is hard to assess with ultrasonography, it certainly appears to be making positive progress after only two weeks.

FIG. 2(a) (before any administration of the composition) shows a nasty SDFT core lesion in the medial aspect not quite involving paratenon. Again the lesion appears to be less hypoechoic on second scan (FIG. 2(b)—after daily administration of the composition for 14 days) suggesting the lesion is filling in with tissue of some sort. Again, the nature and quality of the tissue filling this lesion is hard to assess with ultrasound, but the lesion appears to be making positive progress after only two weeks.

### Conclusions

2 weeks in the field of equine chronic tendon/ligament injuries is a very short timescale and it is rare to see any significant change in these slow healing structures over such a short time period. In more acute injuries, there is a lot more early activity and ultrasonographic evidence of healing as the tendon responds to injury and the inflammatory cascade process commences.

The results presented here appear to show that vitamin A supplementation (by daily administration of the pharmaceutical composition) has had a positive effect on healing of the lesions after only 2 weeks.

### Example 2—Treatment of Equine Connective Tissue Injuries

This example describes the effect of a vitamin A supplement in treating connective tissue injuries in horses.

Connective tissue injuries are common in both human and equine athletes with massive physical, psychological and economic impact. These tendon and ligament injuries tend to take weeks to months of recovery time mainly consisting of rest and rehabilitation, depending on the severity. There are various outcomes possible following injury. Commonly, disorganised scar tissue is formed to replace the native tissue to quickly restore form at the expense of future function. This scar tissue is characterised by a disorganised extracellular matrix which does not have the same mechanical properties and integrity of the original tendon. It follows logically that these athletes are never quite able to achieve optimum performance once scarring has occurred and are also prone to re-injury. It is traditionally believed that once a scar has formed, it is there for life as it is believed to be essentially a passively maintained disorganised cluster of collagen, even after remodelling. However, there is recent evidence that suggests that scar tissue is actively maintained which may present an avenue to target established fibrotic tissue (Fear M et al. Changes in Fibroblast Phenotype and Matrix Turnover in Established Scar Tissue, J Burn Care Res 2019, Vol 40, Page 237).

Vitamin A has multiple functions in animals involving (and not limited to) development, and modulation of protein synthesis, and also possesses anti-inflammatory properties. There is some evidence that vitamin A plays a role in scar tissue formation and maintenance. This example aims to demonstrate the safety, and establish the clinical efficacy, of the usage of vitamin A supplementation in horses with tendon or ligament injuries.

### Methodoloqy

A prospective, single armed pilot trial of the efficacy and safety of Vitamin A supplementation in connective tissue injuries in horses was performed. The time since injury of each horse was noted. Full length ultrasonography of the injured tendon/ligament were performed at baseline as well as 3 weeks, 5 weeks and 7 weeks into the trial. Ultrasonography images were captured at the site of maximal injury. Depending on the nature of the injury, either cross-sectional or longitudinal views were taken. FIG. 3(a) shows a cross-sectional ultrasonogram of the major tendons/ligaments present in the left foot of a horse (normal equine anatomy). FIG. 3(b) shows a longitudinal ultrasonogram (left) of the left foot of a horse compared with a cross-sectional ultrasonogram (right) of the left foot of the same horse (normal equine anatomy). The lesions in the cross-sectional images were measured manually, aided by online software to determine the lesion size as a percentage of the overall cross-sectional area. The longitudinal images were presented to a consultant musculoskeletal radiologist who applied a 5-level grading system of the appearance of the injury corresponding to approximately 0%, 25%, 50%, 75% and 100% lesion size. Side effects and tolerability were also recorded.

### Vitamin A Supplement Used:

Vitamin A palmitate (retinyl palmitate) in a vehicle, delivered in dry feed based on the upper safe concentration in feeds (16,000 IU per kg feed dry matter).

### Administration of Vitamin a Supplement:

Horses with connective tissue injuries were orally administered vitamin A palmitate in a vehicle, at a dose of 160,000 IU vitamin A once per day for 7 weeks. The dose administered was decided based on the known toxic dose in horses (1,000 IU per kg (National Research Council. Nutrient Requirement of Horses: Fifth Revised Edition. The National Academies. 1989)) and the proposed upper safe concentration in feeds (16,000 IU per kg feed dry matter (Ralston S L. Nutritional Requirements of Horses and Other Equids. MSD Veterinary Manual, 2021)) which yielded a dose of 160,000 IU, corresponding to 32% of the toxic dose, assuming a 500 kg horse consuming 10 kg of dry feed. There were no adverse events reported and the supplement was well tolerated by the subjects.

### Subjects:

14 horses with 15 injured limbs were enrolled in the study between March and May 2021. The injuries comprised 9 tendon and 6 ligament injuries, with 12 injuries on the left side of the horse and 3 on the right. The mean time since injury was ~12 months. The tendon injuries comprised 2 acute injuries (<1 month old) and 7 chronic injuries, with a mean time since injury of 13.1 months, and a range of 9-20 months. The ligamentous injuries comprised 6 chronic injuries, with a mean time since injury of 14.3 months and a range of 4-30 months. The most commonly injured structure was the left fore superficial digital flexor tendon (n=8). The most commonly injured ligament was the left fore check ligament. The inclusion criteria were polo horses with diagnosed tendon/ligament injuries regardless of time since injury. There were no exclusion criteria.

### Statistical Analysis:

All statistical analysis was performed on SPSS v27. Missing values were handled by last observation carried forward. Descriptive statistics were used to describe baseline and follow-up values. The results were first tested for normality using the Shapiro-Wilk test. The results at weeks 3, 5 and 7 were then analysed compared to baseline using two-tailed paired Student's t-tests once normality was proven. One-tailed Pearson correlation test was performed to determine if outcomes was correlated with time since injury as we hypothesise that older, more established injuries may benefit less from our supplementation.

### Results

The mean lesion size was 41.44% at baseline, 35.87% at week 3, 28.37% at week 5 and 31.73% at week 7 (FIG. 4(a)). Shapiro-Wilk tests on the data revealed the lesion size was normally distributed at baseline and at weeks 3, 5 and 7. The calculated percentage improvement from baseline to week 7 was also deemed to be normally distributed. Thereafter, paired t-tests showed that the decrease from baseline to weeks 5 and 7 were statistically significant (p=<0.001 and 0.006 respectively) (FIG. 4(a)). When acute injury (<1 month) cases were removed (n=2), similar results were obtained with statistical significance being achieved at weeks 5 and 7 compared to baseline (p=<0.001 and p=0.017 respectively). FIGS. 5 to 16 show ultrasonograms of some of the tendon/ligamentous injuries of the data set at various time points from week 0. Table 1 shows the results of the trial, comprising data for each individual lesion.

One-tailed Pearson correlation coefficient on percentage improvement and time yielded a test statistic of r=−0.508 (p=0.027) indicating a statistically significant negative relationship (FIG. 4(b)). However, removal of 3 outliers yielded r=−0.806 (p=0.003).

TABLE 1
Full results table.

| | Time Since | | | | Initial | Defect Size | Defect | Defect Size | |
|---|---|---|---|---|---|---|---|---|---|
| | Injury at First | Nature of | Site of | | Defect | at 3 weeks | Size at 5 | at 7 weeks | |
| ID | Scan | Injury | Injury | Ultrasound | Size (%) | (%) | weeks (%) | (%) | Comments |
| 1431 | >18 months | Ligament | LF check | Cross- | 45.41 | 35.10 | 32.62 | 29.72 | Full work |
| | | | | sectional | | | | | Movement artifact |
| 3465 | 9 months | Tendon | LF SDFT | Longitudinal | 50.00 | 50.00 | 25.00 | 25.00 | Paddock rest |
| 8827 | >18 months | Tendon | LF SDFT | Cross- | 62.30 | 46.03 | 36.40 | 53.79 | Low level |
| | | | | sectional | | | | | exercise |
| | | | | | | | | | Initial USS had |
| | | | | | | | | | poor contact |
| | | | | | | | | | 6 week f/u |
| | | | | | | | | | movement artifact |
| 10520 | 6 months | Ligament | LH medial | Cross- | 35.97 | 27.49 | 26.19 | 24.26 | Variation in |
| | | | suspensory | sectional | | | | | quality and |
| | | | branch | | | | | | positioning of |
| | | | | | | | | | scans |
| 111112 | >12 months | Tendon | LF SDFT | Cross- | 6.59 | 4.37 | 1.10 | 0.00 | Full work |
| | | | | sectional | | | | | Tendon appears |
| | | | | | | | | | normal, unable to |
| | | | | | | | | | see discrete |
| | | | | | | | | | lesion @ 6 weeks |
| | | | | | | | | | but movement |
| | | | | | | | | | artifact noted |
| 123345 | 9 months | Tendon | LF SDFT | Cross- | 64.93 | 66.51 | 53.75 | 49.16 | Full work |
| | | | | sectional | | | | | Painful clinically |
| 1234567 | 1 month | Tendon | RF lateral | Cross- | 21.78 | 25.82 | 20.30 | 13.03 | Tendon splitting |
| | | | SDFT | sectional | | | | | procedure |
| Q1Q | >12 months | Tendon | LF SDFT | Cross- | 59.28 | 19.29 | 34.02 | 49.75 | Widespread |
| | | | | sectional | | | | | scarring - |
| | | | | | | | | | evolving in tissue |
| | | | | | | | | | density |
| REG6 | 20 months | Tendon | LF SDFT | Cross- | 60.00 | 54.63 | 41.84 | 47.21 | Horse continued |
| | | | | sectional | | | | | exercising hence |
| | | | | | | | | | worsening at |
| | | | | | | | | | week 6 |
| REG7 | >12 months | Tendon | LF SDFT | Longitudinal | 25.00 | 0.00 | 0.00 | 0.00 | |
| REG8 | 20 months | Ligament | LF lateral | Longitudinal | 75.00 | 100.0 | 100.0 | 100.0 | |
| | | | suspensory | | | | | | |
| | | | branch | | | | | | |
| REG9 | Unknown | Ligament | RF lateral | Cross- | 35.36 | 41.97 | 23.84 | 28.60 | |
| | | | suspensory | sectional | | | | | |
| | | | branch | | | | | | |
| 1833 | 4 months | Ligament | LF check | Cross- | 20.11 | 20.11 | 9.17 | 9.17 | |
| | | | | sectional | | | | | |
| 1833 | 30 months | Ligament | RF check | Cross- | 40.00 | 40.00 | 39.54 | 39.54 | Only week 5 |
| | | | | sectional | | | | | |
| 6168 | 1 month | Tendon | LF lateral | Cross- | 19.81 | 6.79 | 6.79 | 6.79 | Only week 3 |
| | | | SDFT | sectional | | | | | |

Note:
LF—left fore, RF—right fore, LH—left hind, RH—right hind, SDFT—superficial digital flexor tendon

### Discussion

The results of the study show that supplementation with vitamin A was extremely well tolerated with no signs of vitamin A toxicity noted in all subjects. Owing to the fact that the subjects were all specialised polo horses, there was a higher incidence of left sided injuries.

Our results show that it takes approximately 5 weeks for there to be significant improvements in the appearance of tendon/ligament injuries in horses which is maintained at least up to week 7 (FIG. 4(a)). Given that more than half of the injuries occurred over 12 months prior to the study and thus were very likely to be stable at the start of the trial, a noticeable improvement in 5 weeks is very astonishing. The results also show that there is a statistically significant linear negative correlation between the time between injury and starting vitamin A supplementation and outcomes (FIG. 4(b)), indicating that earlier vitamin A supplementation leads to better outcomes.

The dataset had three outliers as shown in scatter plot in FIG. 4(b): two at 12 months since injury, which both had 100% improvement, and one at 20 months since injury which had −25% improvement (i.e. the lesion got worse). The explanation for the outlier at 20 months since injury was that the ligamentous injury sustained by the subject was confirmed by a consultant radiologist to be a complete avulsion of the ligament. Short of surgery, there is no possibility of the ligament recovering following that type of injury, including with vitamin A supplement. The two outliers at 12 months since injury both had their lesions become unnoticeable by 7 weeks which is extremely promising.

General wound healing has 4 overlapping stages—haemostasis, inflammation, tissue formation and remodelling. Recent advancements in the field of wound healing suggest that the lack of inflammation in foetal wounds allows it to heal in a scarless manner, restoring the full function, flexibility and architecture of the native tissue (Galatz L M et al. Tendon Regeneration and Scar Formation: The Concept of Scarless Healing. J Orthop Res. 2015; 33:823-31). The known anti-inflammatory properties of vitamin A (Huang Z et al. Role of Vitamin A in the Immune System. J Clin Med. 2018; 7(9):258) support its use in the acute stages of tendon injuries, and indeed has been shown to increase the tensile strength of the healed tendon by double the control at day 45 in a 1990 study on chickens in Greenwald et al. Zone II Flexor Tendon Repair: Effects of Vitamins A, E, β-carotene. J Surg Res. 1990; 49(1):98-102, but does not explain the improvement found in the subjects of our study that have long-standing injuries.

The healing of tendon injuries starts with an early deposition of unoriented collagen fibres. Later on, a dynamic interplay of collagenolysis and deposition of oriented fibres determine the extent of restoration of normal tissue architecture (Greenwald et al., supra). Traditional thinking is that this process plateaus and leaves a permanent fibrotic scar. From our study, the fact that long-standing injuries (>12 months old) showed signs of improvement on ultrasonography is very encouraging especially with the recent suggestion that scar tissue is actively maintained. Fear et al. (supra) suggested this by demonstrating that fibroblasts in scar tissue are phenotypically different to fibroblasts in normal skin which is linked to the difference in matrix turnover.

The main cell type present in tendons are tenocytes (also known as tendon fibroblasts) and these maintain the tendon extracellular matrix ECM. Tendons are characterised by an exceptionally organised, anisotropic extracellular matrix with primarily type I collagen, although small amounts of type Ill collagen are also present (Fratzl P. Collagen: Structure and Mechanics, an Introduction. Collagen. Springer US; 2008. p. 1-13; Kannus P. Structure of the Tendon Connective Tissue. Scand J Med Sci Sport. 2000; 10(6):312-20). Equine tendon scar tissue has been shown to have higher than usual levels of type Ill collagen (20-30%) (Williams I et al. Cell Morphology and Collagen Types in Equine Tendon Scar. Res Vet Sci. 1980; 28:302-10). Vitamin A is well known to play a role in the modulation of the synthesis of extracellular matrix proteins, including collagens, laminins, entactin, fibronectin, elastin and proteoglycans. It also has a role in the expression of various metalloproteinases, including collagenase. As scar tissue is due to excess deposition of disoriented collagen and physiologically abnormal proportions of collagen type by fibroblasts, this may allude to a plausible mechanism how vitamin A may influence fibroblasts maintaining scar tissue to instead produce native tendon tissue.

### Conclusion

In summary, our study found that vitamin A supplementation in horses with established tendon and ligament injuries led to radiological improvement and thus may positively influence the actively maintained characteristic extracellular matrix of scar tissue. We also found evidence that the degree of improvement with this supplementation is correlated with the time since injury. A potential mechanism by which it may act is upon fibroblasts or possibly their progenitor cells, mesenchymal stem cells. As scarring and fibrosis are seen in almost all areas of medicine, these findings have substantial implications and potential therapeutic uses if the mechanistic pathways are found to be more widely applicable.

### Example 3—Treatment of Equine Connective Tissue Injuries

This example describes the pathophysiology of equine tendon/ligament lesions before and after treatment with vitamin A supplement.

### Methodology

Full length ultrasonography of the injured tendon/ligament was performed at the time of initial injury, baseline, as well as 3 weeks, 5 weeks and 7 weeks into the trial. Ultrasonography images were captured at the site of maximal injury. Depending on the nature of the injury, either cross-sectional or longitudinal views were taken. The lesions in these cross-sectional images were measured manually, aided by online software to determine the lesion size as a percentage of the overall cross-sectional area. The longitudinal images were presented to a consultant musculoskeletal radiologist who applied a 5-level grading system of the appearance of the injury corresponding to approximately 0%, 25%, 50%, 75% and 100% lesion size. Side effects and tolerability were also recorded.

The administration of vitamin A supplement, and the vitamin A supplement used, were the same as that of Example 2.

### Subjects:

7 horses with 7 injured limbs were enrolled in the study between March and May 2021. The injuries comprised 3 tendon and 4 ligament injuries. The inclusion criteria were polo horses with diagnosed tendon/ligament injuries regardless of time since injury. The exclusion criteria were horses with acute connective tissue injuries.

### Statistical Analysis:

All statistical analysis was performed on SPSS v27. Missing values were handled by last observation carried forward. Descriptive statistics were used to describe baseline and follow-up values. The results were first tested for normality using the Shapiro-Wilk test. The results at weeks 3, 5 and 7 were then analysed compared to baseline using two-tailed paired Student's t-tests once normality was proven.

### Results

FIG. 17(a) shows the size of the 7 lesions under investigation from the time of original injury, at baseline, and at 3, 5, and 7 weeks into the trial. The mean lesion size was 43.97% at the time of original injury and 49.43% at baseline (time treatment with vitamin A supplement began), but this difference was not statistically significant.

However, when tendon and ligament lesions were treated as two separate cohorts, there was an apparent divergence in natural healing progression between the two tissues from the time of original injury to when treatment began. As shown in FIG. 17(b), the tendons (n=3) tended to get worse as shown by an increase in lesion size across all three tendons, which was statistically significant (mean increase in lesion size=48.7%, p=0.03). Confirming the findings from previous analyses (Example 2), there was a statistically significant improvement in lesion size from baseline to week 7 with treatment with vitamin A supplement (mean improvement in lesion size=30.62% p=0.04).

As shown in FIG. 17(c), the ligaments tended to get better, demonstrated by a reduction in lesion size across three out of four lesions from the time of original injury to the start of treatment. The lesion that increased in size was an extremely severe injury, and the only subject across the entire study that did not improve even after treatment with vitamin A supplement. When analysing all 4 ligamentous injuries, there was no significant difference in lesion size at the time of original injury and the start of the trial (FIG. 17(c)). However, when excluding the known outlier, it was found that there was a statistically significant reduction in lesion size between the time of original injury and the start of the trial (FIG. 17(d)). There was also a reduction in the size of two of the three lesions of the data set from baseline to week 7 with treatment with vitamin A supplement (FIG. 17(d)). The lesion that stayed the same size from week 0 to week 7 was the oldest injury in the trial (30 months since original injury).

### Discussion

Interestingly, it appears that tendons and ligaments behave differently over their natural healing processes with ligamentous lesions either getting better or roughly staying the same from the time of original injury before improving with vitamin A supplement, and tendonous injuries getting significantly worse before improving with vitamin A supplement. These data suggest that there is a physiological difference between tendons and ligaments that results in diverging progression through wound healing, which provides guidance for the next steps.

### Example 4—Effect of Post-Treatment Maintenance Dose of Vitamin a Supplement on Connective Tissue Injuries

This example shows the effect of administering a post-treatment maintenance dose of vitamin A supplement on treating connective tissue injuries in horses. The results provide evidence for continuing at least a maintenance dose of vitamin A supplement past 7 weeks for connective tissue injury healing progression.

### Vitamin A Supplement Used:

Maintenance dose of vitamin A palmitate (retinyl palmitate) in a vehicle, delivered in dry feed at 8,000 IU per kg feed dry matter.

The methodology and subjects of the investigation comprise those of Example 2.

### Administration of Vitamin A Supplement:

At week 7 of full-treatment with vitamin A supplement, 7 horses with connective tissue injuries were orally administered vitamin A palmitate in a vehicle, at a dose of 80,000 IU vitamin A once per day for 7 weeks (half the treatment dose of vitamin A supplement). The known toxic dose in horses is 1,000 IU per kg (National Research Council. Nutrient Requirement of Horses: Fifth Revised Edition. The National Academies. 1989)). The dose of 80,000 IU corresponds to 16% of the toxic dose, assuming a 500 kg horse consuming 10 kg of dry feed. There were no adverse events reported and the supplement was well tolerated by the subjects. The remaining 7 horses were orally administered a placebo in a vehicle, delivered in dry feed. The trial was a double-blind randomised controlled trial.

### Results

The effect of post-treatment maintenance dose of vitamin A supplement on lesion size was investigated. FIG. 18(a) shows the mean lesion size from week 0 to week 7 on full-treatment doses of vitamin A supplement before splitting into the values of the maintenance dose and placebo groups. As described in Example 2, the calculated percentage improvement from baseline to week 7 was deemed to be normally distributed and paired t-tests showed that the decrease from baseline to weeks 5 and 7 were statistically significant (p=<0.001 and 0.006 respectively) (FIG. 4(a)). From week 7 to week 14, the mean lesion size of the placebo group increased from 31.73% to ~35.50%. The mean lesion size of the maintenance dose group decreased from 31.73% to ~20.00% from week 7 to week 14. The data shows that there was continued improvement in the healing of injured connective tissues for subjects that were administered maintenance dose of vitamin A supplement, and deterioration of the lesions for subjects that were administered placebo.

It was possible that the above results may give a slightly biased impression of the effect of the maintenance dose as the subjects in both groups (maintenance vs placebo) had different intragroup mean lesion sizes at week 7. FIG. 18(b) addresses this issue by showing the progression of both groups as well as the average value from Week 0 through to Week 14. Both groups continued to improve from week 7, however the figure still shows a more dramatic improvement in subjects taking a maintenance dose compared to those on placebo (−24.5% vs −6.28%).

One subject became re-injured after treatment with full-treatment doses of vitamin A supplement stopped and a maintenance dose of vitamin A supplement was administered for 7 weeks. FIG. 19 shows an ultrasonogram at baseline (FIG. 21(a)), week 7 (7 weeks of treatment with full-treatment doses of vitamin A supplement) (FIG. 21(b)), and week 14 (7 weeks of treatment with maintenance dose vitamin A supplement) (FIG. 21(c)). The size of the lesion decreases at 7 weeks of full-treatment doses, as shown by a reduction in the hypoechoic area of the ultrasonogram (FIG. 19(b)). However, the lesion becomes much more hypoechoic after post-treatment maintenance dose has been administered for 7 weeks (FIG. 19(c)). Thus, the health of this particular lesion deteriorated after treatment with maintenance dose of vitamin A supplement. This result is different to those described above, wherein the maintenance dose cohort showed improvement in the regeneration of the lesion. The re-injury presented in FIG. 19 provides evidence that, in some cases, treatment with full-treatment doses of vitamin A may be required for longer periods for regeneration of the tissue.

### Example 5

### Techniques for Lesion Size Measurements in Equine Tendons Using Ultrasound

Tendons: attach muscle to bone, transmit forces to elicit movement, and are composed of collagen. In healthy tendons, good parallel alignment allows for optimal force transmission.

Depending on the nature of the injury, either cross-sectional or longitudinal views may be taken. FIG. 3(a) shows a cross-sectional ultrasonogram of the major tendons/ligaments present in the left foot of a horse (normal equine anatomy). FIG. 3(b) shows a longitudinal ultrasonogram (left) of the left foot of a horse compared with a cross-sectional ultrasonogram (right) of the left foot of the same horse (normal equine anatomy). FIG. 20 shows the SDFT and its adjacent DDFT outlined.

Ultrasonographic assessment in injury:
Echogenicity
Tendon size (& lesion size)
Longitudinal fiber alignment
Others (vascularity, shape, etc)

The region of maximal injury (by lesion size) can be captured with ultrasound images.

Measurements taken:
Lesion size (expressed as a percentage of the area of the tendon itself)—see FIG. 23. The lesions in the cross-sectional images are measured manually, aided by online software to determine the lesion size as a percentage of the overall cross-sectional area;

Lesion size—maximal injury zone:
Useful in quantifying extent/severity of injury
May be useful for monitoring progression
But has pitfalls (exact borders difficult to define in healing process)

### Computerised Implementation of Methods Described Herein

Methods are described above in which ultrasonograms or captured ultra sound images (also referred to herein as ultrasonographs) are analysed. As illustrated with reference to FIG. 22, the methods may be implemented on a computer system 100 including a general purpose computer 102, such as a laptop computer, desktop computer, tablet computer, or smart phone as a computerized method. A smart phone is illustrated in FIG. 22. The computerized method may be implemented on a computer or general purpose computer in software such as an application or app. In other words, a computer may be configured to carry out the methods described herein. This reflects the computational efficiency of the methods described herein.

The computer 102 may comprise a processor 104, memory 106 and a display or screen 108, such as a liquid crystal display (LCD) or an organic light emitting diode (OLED) display. The screen may be a touch screen. The computer may also include at least one input port 110 and at least one output port 112.

A computer 102 configured to carry out the methods described herein may be provided with software or computer program code to carry out the method. The software or computer program code may be contained or stored on a computer readable medium, such as a hard-drive, CD-ROM, DVD-ROM, or solid-state memory 106. The computer readable medium may be a non-transitory computer-readable medium.

The ultrasonograph data from an ultra sound scanner scanning the subject may be uploaded to a server 114 or into the cloud. Subsequently, the ultrasonograph may be input into the computer 102 through an input port 110 typically over the Internet through a wireless local area connection such as WiFi or a direct Ethernet connection. Alternatively, the computer may be connected by an input port 110 directly in communication connection to the ultrasound scanner. In which case, the ultrasonograph data is transferred directly from the ultrasound scanner to the computer. The communication connection may be wireless, such as using by short range wireless standard such as Bluetooth. The communication connection may be wired, such as using a Universal Serial Bus (USB) connection. Example USB generations that may be used are USB 2.0, USB 3.0 or USB-C.

In the arrangements described above with reference to, by way of example, FIGS. 1, 2, 3, 5, 6, 7, 8, 9, 10, 11, 12, 13, 14, 15, 16, 19 and 20 lesions are described as being measured including measured manually. When measuring manually, the input ultrasound image of the subject, is displayed on a display 108 of the computer 102, such as the touch screen of a smart phone. This manual measurement may be made by a user tracing an area understood to be a lesion and another area understood to be the overall cross-sectional area of the tendon/ligament by using by moving their finger over the respective areas displayed on the touch screen. The computer then automatically determines the lesion size as a percentage of the overall cross-sectional area of the tendon/ligament. The result of the determination is then displayed on the display of the computer. Alternatively, the measurement may be made automatically by the computer and not manually by a user. In which case, the computer identifies an area understood to be a lesion and another area understood to be the overall cross-sectional area of the tendon/ligament automatically. This analysis is based on a grayscale ultrasound image. The computer determines a portion of the image that is the tendon/ligament by using image segmentation based on the grayscale values of the image. The tendon/ligament has a particular range of grayscale values or intensities. The edges of the lesion are determined using edge detection methods applied to the grayscale ultrasound image. Edge detection methods determine an edge at a position where image brightness changes sharply or there is a discontinuity. Various known edge detection methods may be used such as a search based or zero-crossing based method.

### Example 6

### Techniques for Echogenicity and Lesion Size Measurements in Equine Tendons Using Ultrasound

Tendons: attach muscle to bone, transmit forces to elicit movement, and are composed of collagen. In healthy tendons, good parallel alignment allows for optimal force transmission.

Depending on the nature of the injury, either cross-sectional or longitudinal views may be taken. FIG. 3(a) shows a cross-sectional ultrasonogram of the major tendons/ligaments present in the left foot of a horse (normal equine anatomy). FIG. 3(b) shows a longitudinal ultrasonogram (left) of the left foot of a horse compared with a cross-sectional ultrasonogram (right) of the left foot of the same horse (normal equine anatomy). FIG. 20 shows the SDFT and its adjacent DDFT outlined.

Ultrasonographic assessment in injury:
Echogenicity
Tendon size (& lesion size)
Longitudinal fiber alignment
Others (vascularity, shape, etc)

The region of maximal injury (by lesion size) can be captured with ultrasound images.

### Measurements Taken:

1) Lesion size (expressed as a percentage of the area of the tendon itself)—see FIG. 23. The lesions in the cross-sectional images are measured manually, aided by online software to determine the lesion size as a percentage of the overall cross-sectional area;
2) Echogenicity of tendons—see FIG. 24:
   Using grey scale statistics;
   Compared to healthy adjacent tendon (DDFT) (to account for different gain settings)
3) Echogenicity of tendons—see FIG. 25:
   SDFT vs DDFT

The echogenicity of an injured tendon is assessed by comparing grey scale statistics of the tendon with the values of a healthy adjacent tendon tissue. Due to equine anatomy, there is always a directly adjacent healthy tendon to use as a control (ultrasonography is not very affected by depth of tissue). A quantitative measure of echogenicity of each structure (for example, SDFT and DDFT) is computed for every image. This is performed by grey scale analysis, for example using the open-source image processing software, ImageJ2 (version 2.3.0/1.53f). The mean grey scale (MGS) values of each of the two structures are collected. This produces a value between 0 (black) and 255 (white), acting as a proxy for the echogenicity of the tendon. An SDFT:DDFT ratio is produced from the resultant values of each image for each limb (for example, SDFT/DDFT). The ratio of mean echogenicity of the injured tendon to that of the healthy tendon is determined to assess the extent of damage/healing at the lesion.

Lesion size—maximal injury zone:
Useful in quantifying extent/severity of injury
May be useful for monitoring progression
But has pitfalls (exact borders difficult to define in healing process)

Echogenicity:
of core/main lesion;
of surrounding tendon;
proxy for tissue composition (Tsukiyama et al 1996—scar tissue & injured tendons were hypoechoic relative to healthy tendon—correlated histologically). Change in echogenicity implies change in composition of tissue.

### Example 7

Treatment of Equine Connective Tissue Injury with Vitamin a Supplement and Platelet-Rich Plasma (PRP)

This example describes the effect of a combination therapy of vitamin A supplement with PRP in treating equine connective tissue injury.

### Vitamin a Supplement Used:

Vitamin A palmitate (retinyl palmitate) in a vehicle, delivered in dry feed at 16,000 IU per kg feed dry matter (full-dose treatment) or 8,000 IU per kg feed dry matter (maintenance dose treatment).

### Administration of Vitamin a Supplement:

Full-dose treatment: oral administration of vitamin A palmitate in a vehicle, at a dose of 160,000 IU vitamin A once per day.

Maintenance dose treatment: oral administration of vitamin A palmitate in a vehicle, at a dose of 80,000 IU vitamin A once per day.

### Methodology:

Rania (a female horse) sustained a tendon injury in April 2021 and was started on full-dose treatment with vitamin A for 7 weeks. She improved tremendously well in both lesion size and echogenicity, achieving nearly full resolution of her injury. After her initial treatment period of 7 weeks, she was administered a maintenance dose (half-dose) of vitamin A. Almost immediately after halving her dose, in week 8, she sustained an horrendous reinjury, and was placed back on full-dose treatment with vitamin A. Rania was administered PRP at week 11. She was box rested from when she sustained here original injury, but started full work from week 20.

Scans were taken at the following time points:
1. Week 0 (full-dose treatment vitamin A supplement commenced);
2. Week 3;
3. Week 7 (maintenance dose treatment with vitamin A supplement);
4. Week 8 (Reinjury, full-dose treatment with vitamin A supplement restored);
5. Week 11 (PRP administered, whilst full-dose treatment with vitamin A supplement continued);
6. Week 20 (full work commenced);
7. Week 24.

Ultrasonography images were captured of the injured tendon. To determine the lesion size as a percentage of the entire cross-sectional area of the tendon, the lesion in the ultrasonogram image was measured manually, aided by online software. The echogenicity of the injured tendon was assessed by comparing grey scale statistics of the tendon with the values of a healthy adjacent tendon tissue. Due to equine anatomy, there is always a directly adjacent healthy tendon to use as a control (ultrasonography is not very affected by depth of tissue). The statistics comprised determining the mean pixel intensity at a number of random points on the injured tendon and the healthy tendon, and calculating a mean value for the echogenicity of each tissue. A ratio of mean echnogenicity of the injured tendon and the healthy tissue was determined to assess the extent of damage/healing at the lesion.

### Results

The results are shown in FIG. 26. Lesion size (left hand axis) is represented in green (shown as the top line at the y axis for lesion size). Echogenicity values (right hand axis) are shown in blue and red. The blue line (shown as the bottom line at the y axis for lesion size) shows a plot of the values for the ratio of lesion:healthy tissue. This provides an assessment of the echogenicity of the main lesion. The red line (shown as the middle line at the y axis for lesion size) shows a plot of the values for the ratio of injured:healthy tissue. This provides an assessment of the echogenicity of the rest of the injured tendon.

The plots in FIG. 26 show that, over the first 7 weeks of full-dose treatment with vitamin A supplement, the lesion size decreased dramatically from just under 20% to ~2.5%. The values for the echogenicity ratios also increased over this period, achieving near full resolution of her injury. Following reinjury (week 8), the lesion size increased to 21.52% of the tendon, and the values for the echogenicity ratios fell to ~0.350 and ~0.750 for the blue and red lines, respectively. At week 11 (3 weeks after restarting full-dose treatment), the lesion size was similar, at 20.62%, but with considerable improvements in echogenicity values (~0.900 and ~1.050 for the blue and red lines, respectively).

By week 20 (~9 weeks after administration of PRP), the lesion size had shrunk considerably, to 2.01% of the entire tendon, although the values for the echogenicity ratios appeared to have slightly deteriorated (~0.700 and ~0.900 for the blue and red lines, respectively), but still showed substantial improvement compared to baseline. Rania was able to start full work again from week 20.

The scan at week 24 shows that the lesion size measures 2.19%, which is stable from the scan at week 20 (showing 2.01%). The echogenicity of the main lesion improved (demonstrated by the blue line in FIG. 26) and the echogenicity of the rest of the injured tendon remained stable (see the red line in FIG. 26). By this stage, the benefits from the combination therapy may largely be plateauing, although there may still be some remodelling of the main scar tissue. The values which are plateauing are almost identical to the values achieved at week 7 (just before the re-injury).

### Conclusions

It was concluded from these results that full-dose treatment with vitamin A supplement resulted in substantial healing of the original tendon injury. Despite a nasty reinjury, combination treatment with PRP and vitamin A supplement resulted in improvement up to the same point as before, even though a reinjury would not be expected to recover as well.

These results appear to support an interpretation that administration of PRP resets the injury healing process, as shown by the apparent ‘worsening’ echogenicity by week 20 accompanied by substantial reduction in lesion size (~20% to ~2%). An alternative explanation for the mild worsening of echogenicity is that tissue regeneration was hindered due to the lack of physical stimulus as Rania was box rested.

The synergy between the vitamin A and PRP combination treatment seems to facilitate the lesion to achieve continued regeneration of the injured tissue more so than just vitamin A or PRP alone.

### Example 8

### Treatment of Equine Tendon Injury

This example shows the effect of vitamin A supplement on the echogenicity and health of tendon injury.

Structures composed of different tissue will have different echogenicities. The health of a tissue such as a tendon can be assessed by comparing the echogenicity of the tissue with that of a corresponding healthy tissue. Healthy tendons comprising native tissue and normal architecture are usually hyperechoic and appear white on the sonogram; they are capable of reflecting ultrasound that is cast over the tissue. An injured tendon comprising a lesion will appear as less hyperechoic, and more hypoechoic as tendon fibres are interrupted and defects are usually filled with fluid, blood, or fat. Severe lesions will be anechoic, and will display as completely dark on the sonogram.

### Methodoloqy:

Full length ultrasonography of the injured tendon and adjacent healthy tendon was performed at baseline as well as 3 weeks, 5 weeks and 7 weeks into the trial. Ultrasonography images were captured at the site of maximal injury of the injured tendon. Depending on the nature of the injury, either cross-sectional or longitudinal views were taken. The echogenicity of a tendon lesion was assessed by comparing grey scale statistics of the tendon lesion with the values of healthy adjacent tendon tissue (FIG. 27(b)). Due to equine anatomy, there is always a directly adjacent healthy tendon to use as a control (ultrasonography is not very affected by depth of tissue). The statistics comprised determining the mean pixel intensity at a number of random points on the lesion and the healthy tendon, and calculating a mean value for the echogenicity of each tissue. A ratio of mean echogenicity of the lesion and the healthy tissue was determined to assess the extent of damage/healing at the lesion.

### Vitamin A Supplement Used:

Vitamin A palmitate (retinyl palmitate) in a vehicle, delivered in dry feed based on the upper safe concentration in feeds (16,000 IU per kg feed dry matter).

### Administration of Vitamin A Supplement:

Horses with connective tissue injuries were orally administered vitamin A palmitate in a vehicle, at a dose of 160,000 IU vitamin A once per day for 7 weeks. The dose administered was decided based on the known toxic dose in horses (1,000 IU per kg (National Research Council. Nutrient Requirement of Horses: Fifth Revised Edition. The National Academies. 1989)) and the proposed upper safe concentration in feeds (16,000 IU per kg feed dry matter (Ralston S L. Nutritional Requirements of Horses and Other Equids. MSD Veterinary Manual, 2021)) which yielded a dose of 160,000 IU, corresponding to 32% of the toxic dose, assuming a 500 kg horse consuming 10 kg of dry feed. There were no adverse events reported and the supplement was well tolerated by the subjects.

### Subjects:

14 horses with 15 injured limbs were enrolled in the study between March and May 2021. The injuries comprised 9 tendon and 6 ligament injuries, with 12 injuries on the left side of the horse and 3 on the right. The mean time since injury was ~12 months. The tendon injuries comprised 2 acute injuries (<1 month old) and 7 chronic injuries, with a mean time since injury of 13.1 months, and a range of 9-20 months. The ligamentous injuries comprised 6 chronic injuries, with a mean time since injury of 14.3 months and a range of 4-30 months. The most commonly injured structure was the left fore superficial digital flexor tendon (n=8). The most commonly injured ligament was the left fore check ligament. The inclusion criteria were polo horses with diagnosed tendon/ligament injuries regardless of time since injury. There were no exclusion criteria.

### Results

The mean echogenicity ratio of the lesion and healthy tendon increased each week from baseline to week 7 (FIG. 27(a)), which was statistically significant. A value of 1 would indicate perfect regeneration of native tendon. The mean echogenicity ratio was 0.52 at baseline, and 0.69 at week 7, equating to a % increase of 32.7%. As shown in FIG. 27(a), the mean echogenicity ratio from baseline to week 7 showed a continuing positive gradient. This shows that the lesion is being replaced with native tendon tissue at a constant rate, providing evidence for continuing trials with full dose of vitamin A supplement past 7 weeks to achieve even more regeneration of the tendon. FIG. 27(b) shows an outline of the whole injured tendon and area of lesion. FIG. 27(c) shows an outline of an adjacent healthy tendon (as well as the outline of the injured tissue and lesion) used as a comparison tissue.

Interestingly, the injured tendon had lower echogenicity than the healthy tendon when the area of lesion was excluded and the remaining area of the injured tendon was examined (FIG. 28(b). FIG. 28(a) shows that the mean echogenicity ratio at week 0 was 0.76, which increased marginally to 0.82 at week 7, which was statistically significant. It was observed that the lesion size had normalised to the size of the tendon itself, suggesting that the lesion was not contained to the point of maximal injury, but that the health of the whole tendon was impaired.

### Example 9

### Left-Right Tendon Echogenicity Ratios in Uninjured Polo Ponies and Racehorses

### Background

There exist many disciplines within elite equine athletes, each with their own sport-specific training regimes and demands. Polo and horse racing are two such sports. Notably, polo ponies are exercised in a clockwise direction and trained to canter with a right forelimb lead owing to the right handed nature of polo. This is in contrast to racehorses, which are trained in both clockwise and anticlockwise directions in the UK, and take both left and right forelimb leads. It has been shown previously that the force transmitted through the lead and non-lead limbs are different, with the non-lead limb experiencing higher forces in canter (1). The outside limb in a circular track has also been shown to experience higher forces (2).

It is hypothesised that the difference in training patterns in polo ponies and racehorses, in that polo ponies preferentially takes a right forelimb lead, there would be a difference in the echogenicities of the superficial digital flexor tendon and deep digital flexor tendon in the left and right forelimbs in polo ponies but not in racehorses.

### Methods

A cross-sectional study was performed in the UK, investigating the relationship of the SDFT to DDFT in both left and right forelimbs of polo ponies and racehorses.

### Subjects

Equine athletes were randomly selected to participate in the study: 5 healthy polo ponies and 9 healthy racehorses. They were recruited by the senior author. The inclusion criteria were high performance equine athletes currently training for their respective sports with a regular training schedule. The sole exclusion criterion was any history of musculoskeletal injury.

Cross-sectional ultrasonographic images of both the right and left forelimbs of each subject were taken at regular intervals throughout the length of the limb, producing 7 cross-sectional images for each limb. The ultrasound machine setting remained constant when measuring each subject.

A quantitative measure of echogenicity of each structure (SDFT and DDFT) was computed for every image. This was performed via grey scale analysis, using the open-source image processing software, ImageJ2 (version 2.3.0/1.53f). The mean grey scale (MGS) values of each of the two structures were collected. This produced a value between 0 (black) and 255 (white), acting as a proxy for the echogenicity of the tendon. An SDFT:DDFT ratio was produced from the resultant values of each image for each limb (SDFT/DDFT). The mean SDFT:DDFT ratio for the limb was then calculated from all 7 images.

### Statistical Analysis

All statistical analysis was performed on SPSS version 27. A power calculation was performed to show a SDFT:DDFT left-right difference of 0.05 with a power of 80%. The distributions were tested for normality using the Shapiro-Wilk test. The continuous variables, left and right SDFT:DDFT MGS ratios, were compared using paired t-tests to establish significance. A p value of <0.05 was considered significant.

### Results

### Polo Ponies

All 5 subjects had images of adequate quality and the echogenicity of all SDFT and DDFT tendons were measured. The summary of results is shown in Table 1. The overall mean of the SDFT:DDFT ratio was 0.91 on the left forelimb and 0.86 on the right forelimb. A point to note is that the SDFT:DDFT ratios were always greater on the left than right side. This difference was statistically significant (p=0.008).

TABLE 1
Summary of mean SDFT:DDFT ratios by limb in each polo pony.

| | Mean SDFT:DDFT ratio | | |
|---|---|---|---|
| | Left | Right | Difference |
| Polo Pony 1 | 0.96 | 0.90 | +0.06 |
| Polo Pony 2 | 0.88 | 0.85 | +0.03 |
| Polo Pony 3 | 0.92 | 0.85 | +0.07 |
| Polo Pony 4 | 0.88 | 0.83 | +0.05 |
| Polo Pony 5 | 0.91 | 0.89 | +0.02 |
| Mean | 0.91 | 0.86 | +0.05 |

### Racehorses

All 9 subjects had images of adequate quality and the echogenicity of all SDFT and DDFT tendons were measured. The summary of results is shown in Table 2. The overall mean SDFT:DDFT ratios of both the left and right forelimbs were 0.78. The results were not statistically significantly different (p=0.78).

TABLE 2
Summary of mean SDFT:DDFT ratios by limb in each racehorse.

| | Mean SDFT:DDFT ratio | | |
|---|---|---|---|
| | Left | Right | Difference |
| Racehorse 1 | 0.87 | 0.84 | +0.03 |
| Racehorse 2 | 0.82 | 0.83 | −0.01 |
| Racehorse 3 | 0.73 | 0.73 | +0.01 |
| Racehorse 4 | 0.69 | 0.76 | −0.07 |
| Racehorse 5 | 0.83 | 0.78 | +0.05 |
| Racehorse 6 | 0.80 | 0.76 | +0.03 |
| Racehorse 7 | 0.79 | 0.78 | +0.01 |
| Racehorse 8 | 0.80 | 0.77 | +0.03 |
| Racehorse 9 | 0.71 | 0.75 | −0.04 |
| Mean | 0.78 | 0.78 | 0.00 |

### Discussion

The SDFT is an energy-storing tendon, analogous to the Achilles tendon in humans, whereby it stretches and recoils with each stride to reduce the energy requirements of movement by up to 36%(3,4). As a result of its function, it experiences particularly high stresses and strains during exercise(5), making it prone to injury—75-95% of equine tendon injuries occurring in the forelimb SDFT(6). The result of SDFT injuries are devastating—mature adult tendons form fibrotic scars following injuries as they seem to be unable to remodel the structure of these injured tendons to their pre-injury state (4). These scarred tendons inherently have less desirable biomechanical properties owing to their disorganised nature, making the athletes susceptible to reinjury rates of around 42% with conservative treatment (7).

Although ultrasonography is widely used for the assessment of SDFT injuries, a limiting factor for the interpretation of echogenicity from this modality is the fact that minor changes in settings or operator method result in inconsistencies of image quality. Our novel method of quantifying echogenicity of the tendons by normalising MGS values of the SDFT to DDFT overcomes this limitation.

Our results demonstrate a measurable and statistically significant difference in the SDFT:DDFT ratios between the left and right forelimbs in polo ponies (0.05, p=0.008). This difference was not seen in the racehorse sample. To our knowledge, this is the first such study demonstrating a quantified left-right differential in echogenicity of the flexor tendons in an equine population. One possible explanation for this left-right differential found in polo ponies but not racehorses is the loading experienced by the tendons during training.

Polo ponies are exercised in circular tracks, most commonly performed in a clockwise direction to encourage a right lead (8). The reason for this is that all the main governing bodies of the sport of polo mandates the use of the mallet in the players' right hand (9,10) for safety purposes. It has been shown that the non-lead limb experiences higher forces in canter (1) and the outside limb experiences higher forces in trot (2). This means that the left forelimb experiences greater repetitive forces during the training periods in polo ponies.

Conversely, racehorses train and compete in both clockwise and anticlockwise directions in the UK. It is also known that racehorses regularly switch forelimb leads in races and training. A reasonable assumption would be that both left and right forelimbs experience similar amounts of repetitive loading throughout the racehorses' career, but no empirical evidence of this is available.

However, in Australia and Hong Kong, thoroughbreds are noted to be trained and raced in exclusively clockwise directions along the racetrack(11). Our study did not include racehorses trained in these countries due to its exploratory nature, however further studies should include these horses.

This study is not without its limitations. The major one would be the small sample size of equine athletes from the same country, which although was calculated to provide adequate power, should be validated on a more widespread level. Secondly, the exact contribution of each SDFT and DDFT cannot be determined i.e. is it the SDFT that is more hyperechoic or the DDFT that is more hypoechoic on the left than the right forelimbs of polo ponies?Additionally, although we demonstrated a difference in echogenicity, we cannot determine the composition of the tendon from this.

One question from this study is whether the results are indeed an expected physiological consequence of and/or adaptation to the nature of polo training?

The quantification of echogenicity described in this example could be used as a predictor of future injury, i.e. there exists a normal range expected for the left-right SDFT:DDFT differential, and athletes outside of this range could be predisposed to tendon injury. Any left-right SDFT:DDFT differential could provide evidence of microinjury and thus predispose to injury on that side. This can be further investigated by conducting gait analysis to quantify movement symmetry as Pfau et al. did (8) and correlate with ultrasonographic echogenicity findings.

### Conclusion

In this cross-sectional study, we demonstrate a left-right differential in the echogenicity of the SDFT compared to DDFT in polo ponies but not in UK racehorses. This was done using a novel method of quantifying echogenicity from ultrasonographic images. Further studies need to be done to establish the significance and underlying cause of these findings with implications of early detection of impending injuries.

### Computerised Implementation of Methods Described Herein

Methods are described above in which ultrasonograms or captured ultra sound images (also referred to herein as ultrasonographs) are analysed. As illustrated with reference to FIG. 22, the methods may be implemented on a computer system 100 including a general purpose computer 102, such as a laptop computer, desktop computer, tablet computer, or smart phone as a computerized method. A smart phone is illustrated in FIG. 22. The computerized method may be implemented on a computer or general purpose computer in software such as an application or app. In other words, a computer may be configured to carry out the methods described herein. This reflects the computational efficiency of the methods described herein.

The computer 102 may comprise a processor 104, memory 106 and a display or screen 108, such as a liquid crystal display (LCD) or an organic light emitting diode (OLED) display. The screen may be a touch screen. The computer may also include at least one input port 110 and at least one output port 112.

A computer 102 configured to carry out the methods described herein may be provided with software or computer program code to carry out the method. The software or computer program code may be contained or stored on a computer readable medium, such as a hard-drive, CD-ROM, DVD-ROM, or solid-state memory 106. The computer readable medium may be a non-transitory computer-readable medium.

The ultrasonograph data from an ultra sound scanner scanning the subject may be uploaded to a server 114 or into the cloud. Subsequently, the ultrasonograph may be input into the computer 102 through an input port 110 typically over the Internet through a wireless local area connection such as WiFi or a direct Ethernet connection. Alternatively, the computer may be connected by an input port 110 directly in communication connection to the ultrasound scanner. In which case, the ultrasonograph data is transferred directly from the ultrasound scanner to the computer. The communication connection may be wireless, such as using by short range wireless standard such as Bluetooth. The communication connection may be wired, such as using a Universal Serial Bus (USB) connection. Example USB generations that may be used are USB 2.0, USB 3.0 or USB-C.

In the arrangement described above with reference to FIG. 23, lesions are described as being measured manually. In this case, the input ultrasound image of the subject, is displayed on a display 108 of the computer 102, such as the touch screen of a smart phone. This manual measurement may be made by a user tracing an area understood to be a lesion and another area understood to be the overall cross-sectional area of the tendon/ligament by using by moving their finger over the respective areas displayed on the touch screen. The computer then automatically determines the lesion size as a percentage of the overall cross-sectional area of the tendon/ligament. The result of the determination is then displayed on the display of the computer. Alternatively, the measurement may be made automatically by the computer and not manually by a user. In which case, the computer identifies an area understood to be a lesion and another area understood to be the overall cross-sectional area of the tendon/ligament automatically. This analysis is based on a grayscale ultrasound image. The computer determines a portion of the image that is the tendon/ligament by using image segmentation based on the grayscale values of the image. The tendon/ligament has a particular range of grayscale values or intensities. The edges of the lesion are determined using edge detection methods applied to the grayscale ultrasound image. Edge detection methods determine an edge at a position where image brightness changes sharply or there is a discontinuity. Various known edge detection methods may be used such as a search-based or zero-crossing based method.

In the arrangement described above with reference to FIG. 24, the computer 102 automatically uses grayscale statistics to determine the echogenicity of the tendons. In grayscale statistics, a grayscale ultrasound image is analysed. A particular range of grayscale values or intensities indicate echogenicity when compared to a particular range of grayscale values or intensities associated with a healthy tendon/ligament. The determined echogenicity, echogenicity value and/or fitness potential is displayed on the display 108 of the computer. Alternatively or additionally, the determined echogenicity, echogenicity value and/or fitness potential may be saved or stored in a memory or other storage device of the computer. Alternatively or additionally, the determined echogenicity, echogenicity value and/or fitness potential may be output to an external store such as a server 114 from an output port 112.

### REFERENCES

1. Witte T H, Knill K, Wilson A M. Determination of peak vertical ground reaction force from duty factor in the horse (Equus caballus). J Exp Biol. 2004; 207(21):3639-48.
2. Chateau H, Camus M, Holden-Douilly L, Falala S, Ravary B, Vergari C, et al. Kinetics of the forelimb in horses circling on different ground surfaces at the trot. Vet J [Internet]. 2013; 198(SUPPL1):e20-6. Available from: http://dx.doi.org/10.1016/j.tvjl.2013.09.028
3. Biewener A A. Muscle-tendon stresses and elastic energy storage during locomotion in the horse. Comp Biochem Physiol—B Biochem Mol Biol. 1998; 120(1):73-87.
4. O'Brien C, Marr N, Thorpe C. Microdamage in the equine superficial digital flexor tendon. Equine Vet J. 2021; 53(3):417-30.
5. Patterson-Kane J C, Becker D L, Rich T. The Pathogenesis of Tendon Microdamage in Athletes: The Horse as a Natural Model for Basic Cellular Research. J Comp Pathol [Internet]. 2012; 147(2-3):227-47. Available from: http://dx.doi.org/10.1016/j.jcpa.2012.05.010
6. Thorpe C T, Clegg P D, Birch H L. A review of tendon injury: Why is the equine superficial digital flexor tendon most at risk?Equine Vet J. 2010; 42(2):174-80.
7. Dyson S J. Medical management of superficial digital flexor tendonitis: A comparative study in 219 horses (1992-2000). Equine Vet J. 2004; 36(5):415-9.
8. Pfau T, Parkes R S, Burden E R, Bell N, Fairhurst H, Witte T H. Movement asymmetry in working polo horses. Equine Vet J. 2016; 48(4):517-22.
9. United States Polo Association. Rulebook of the United States Polo Association 2020. 2020;
10. Hurlingham Polo Association. Rules & Regulations 2021. 2021; Available from: http://www.hpa-polo.co.uk/wp-content/uploads/2014/04/HPA-Rules-2014.pdf
11. Williams D E, Norris B J. Laterality in stride pattern preferences in racehorses. Anim Behav. 2007; 74(4):941-50.

1. A method for determining a size of a lesion in a connective tissue, which comprises:
i) determining an area of the lesion from an ultrasonogram of an ultrasound scan carried out on the connective tissue, and determining a total area of the connective tissue from the ultrasonogram; and
ii) determining the area of the lesion as a proportion of the total area of the connective tissue.

2. A method according to claim 1, wherein, if the area of the lesion comprises:
i) 0-15% of the total area of the connective tissue, determining that the lesion is mild;
ii) 16-25% of the total area of the connective tissue, determining that the lesion is moderate; or
iii) >25% of the total area of the connective tissue, determining that the lesion is severe.

3. A method according to claim 2, wherein a treatment program is initiated for the subject which is appropriate for the size and/or severity of the lesion.

4. A method according to claim 3, wherein the treatment program comprises administering to the subject anti-inflammatory medication, dietary supplementation, or stem-cell treatment, and/or cold therapy, rest, confinement, surgery, or a loading/working training regime.

5. A method according to any preceding claim, which further comprises repeating steps (i) and (ii) of claim 1 after a period to determine whether there is a change in the area of the lesion as a proportion of the total area of the connective tissue.

6. A method according to claim 5, wherein the period is at least one day, at least a week, at least two weeks, at least a month, at least six months, or at least twelve months.

7. A method according to claim 5 or 6, wherein if it is determined that the area of the lesion as a proportion of the total area of the connective tissue has decreased over the period, determining that the lesion is healing.

8. A method according to claim 5 or 6, wherein if it is determined that the area of the lesion as a proportion of the total area of the connective tissue has increased over the period, determining that the lesion is progressing.

9. A method for monitoring a change in a size of a lesion in a connective tissue, which comprises:
i) determining an area of the lesion from an ultrasonogram of an ultrasound scan carried out on the connective tissue, and determining a total area of the connective tissue from the ultrasonogram;
ii) determining the area of the lesion as a proportion of the total area of the connective tissue;
iii) repeating steps (i) and (ii) after a period; and
iv) determining whether the area of the lesion as a proportion of the total area of the connective tissue has changed.

10. A method according to claim 9, wherein if it is determined that the area of the lesion as a proportion of the total area of the connective tissue obtained in step (iii) is less than that obtained in step (ii), determining that the lesion size has decreased over the period.

11. A method according to claim 10, wherein it is determined that the lesion is healing.

12. A method according to claim 9, wherein if it is determined that the area of the lesion as a proportion of the total area of the connective tissue obtained in step (iii) is greater than that obtained in step (ii), determining that the lesion size has increased over the period.

13. A method according to claim 12, wherein it is determined that the lesion is progressing.

14. A method according to claim 9, wherein if it is determined that the area of the lesion as a proportion of the total area of the connective tissue obtained in step (iii) is the same as that obtained in step (ii), determining that the lesion size has remained the same over the period.

15. A method according to claim 14, wherein it is determined that the lesion is stable.

16. A method according to any of claims 9 to 15, wherein the period is at least one day, at least a week, at least two weeks, at least a month, at least six months, or at least twelve months.

17. A method according to any preceding claim, which further comprises carrying out the ultrasound scan of the connective tissue, and providing an ultrasonogram from the ultrasound scan.

18. A method according to claim 17, wherein the ultrasonogram is a cross-sectional ultrasonogram.

19. A method according to claim 17, wherein the ultrasonogram is a longitudinal ultrasonogram.

20. A method according to any preceding claim, wherein the area of the lesion is determined by grey scale analysis of the ultrasonograms.

21. A method according to any preceding claim, wherein an area of the connective tissue which does not form a part of the area of the lesion is healthy connective tissue.

22. A method according to any preceding claim wherein the connective tissue is a tendon or ligament tissue.

23. A method according to any preceding claim, wherein the lesion is the result of an injury to the subject.

24. A method according to claim 23, wherein the injury is an acute injury.

25. A method according to claim 23 or 24, wherein the injury is a traumatic injury.

26. A method according to claim 23, wherein the injury is an overload injury.

27. A method according to claim 23, wherein the injury is a chronic injury.

28. A method according to claim 27, wherein the chronic injury is caused by long-term micro-trauma or degradation of the connective tissue.

29. A method according to claim 27 or 28, wherein the chronic injury is a caused by chronic inflammation about the tissue.

30. A method according to any preceding claim, wherein the subject is a horse.

31. A method according to claim 30, wherein the subject is a polo horse.

32. A method according to claim 31, wherein the polo horse takes a right forelimb lead.

33. A method according to claim 30, wherein the subject is a racehorse.

34. A method according to claim 33, wherein the racehorse takes a right or a left forelimb lead.

35. A method according to any of claims 30 to 34, wherein the connective tissue comprises a flexor tendon or an extensor tendon.

36. A method according to claim 35, wherein the flexor tendon is a superficial digital flexor tendon (SDFT), or a deep digital flexor tendon (DDFT).

37. A method according to claim 35, wherein the extensor tendon is a lateral digital extensor tendon (LDET), or a common digital extensor tendon (CDET).

38. A method according to any of claims 30 to 34, wherein the connective tissue comprises a suspensory ligament, or a check ligament.

39. A method according to any of claims 1 to 29, wherein the subject is a non-human subject.

40. A method according to any of claims 1 to 29, wherein the subject is a human subject.

41. A method according to claim 40, wherein the connective tissue comprises an anterior cruciate ligament (ACL).

42. A method according to claim 40 wherein the connective tissue comprises an Achilles tendon.

43. A method according to claim 40 wherein the connective tissue comprises an extensor tendon.

44. A method according to claim 40 wherein the connective tissue comprises a flexor tendon.

45. A method according to claim 40, wherein the connective tissue comprises a tibial tendon.

46. A method according to claim 40, wherein the connective tissue comprises a peroneal tendon.

47. A method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a ratio of the echogenicity value of the first connective tissue to the echogenicity value of the second tissue; and
iii) if the ratio determined in step (ii) is:
a) less than one, determining that the fitness potential of the connective tissue of interest is lower than the fitness potential of the second connective tissue; or
b) greater than one, determining that the fitness potential of the connective tissue of interest is higher than the fitness potential of the second connective tissue; or
c) one, determining that the fitness potential of the connective tissue of interest is the same as the fitness potential of the second connective tissue.

48. A method according to claim 47, wherein the second connective tissue is healthy connective tissue, and in step (iii), if the ratio determined in step (ii) is:
a) less than one, determining that the connective tissue of interest is injured or at risk of injury; or
b) greater than one, determining that the connective tissue of interest has a high fitness potential; or
c) one, determining that the connective tissue of interest is healthy.

49. A method according to claim 47 or 48, which further comprises repeating steps (i) to (iii) after a period to determine whether there is a change in the fitness potential of the connective tissue of interest.

50. A method according to claim 49, wherein the period is at least one day, at least a week, at least two weeks, or at least a month.

51. A method for determining a change in the fitness potential of a connective tissue of interest in a subject, the method comprising:
i) determining an echogenicity value of a first connective tissue in a limb of the subject, and an echogenicity value of a healthy second connective tissue in the same limb of the subject, wherein the connective tissue of interest is the first connective tissue;
ii) determining a ratio of the echogenicity value of the first connective tissue to the echogenicity value of the second tissue; and
iii) repeating steps (i) and (ii) after a period; and
iv) if the ratio obtained in step (iii) is:
a) higher than the ratio obtained in step (ii), determining that the fitness potential of the connective tissue of interest has increased; or
b) similar to the ratio obtained in step (ii), determining that the fitness potential of the connective tissue of interest has remained the same; or
c) lower than the ratio obtained in step (ii), determining that the fitness potential of the connective tissue of interest has reduced.

52. A method according to claim 51, wherein the period is at least one day, at least a week, at least two weeks, or at least a month.

53. A method according to claim 51 or 52, wherein the connective tissue of interest is an injured connective tissue, and if the ratio obtained in step (iii) is higher than the ratio obtained in step (ii), it is determined that the injury to the connective tissue of interest is healing.

54. A method according to claim 51 or 52, wherein the connective tissue of interest is a healthy connective tissue, and if the ratio obtained in step (iii) is lower than the ratio obtained in step (ii), it is determined that the connective tissue of interest is at risk of injury.

55. A method for determining the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a relative difference between the echogenicity values obtained for the first and second connective tissues in step (i);
iii) comparing the relative difference obtained in step (ii) with a corresponding relative difference obtained for first and second connective tissue in a limb of the same or a different subject; and
iv) if the relative difference obtained in step (ii) is:
a) lower than the compared relative difference in step (iii), determining that the fitness potential of the connective tissue of interest is lower than the fitness potential of the first connective tissue in the compared limb; or
b) similar to the compared relative difference in step (iii), determining that the fitness potential of the connective tissue of interest is similar to the fitness potential of the first connective tissue in the compared limb; or
c) higher than the compared relative difference in step (iii), determining that the fitness potential of the connective tissue of interest is higher than the fitness potential of the first connective tissue in the compared limb.

56. A method according to claim 55, wherein the compared limb in step (iii) is a different limb of the same subject.

57. A method according to claim 55, wherein the compared limb in step (iii) is the same limb of a different subject.

58. A method according to claim 55, wherein the compared limb in step (iii) is a different limb of a different subject.

59. A method according to any of claims 55 to 58, wherein the corresponding relative difference obtained for first and second connective tissue of the compared limb in step (iii) is a predetermined corresponding relative difference previously obtained for the first and second connective tissues of the compared limb.

60. A method according to claim 59, wherein the corresponding relative difference obtained for first and second connective tissue in step (iii) is a predetermined corresponding relative difference previously obtained for the first and second connective tissues in the same limb of the same subject.

61. A method according to claim 60, wherein the predetermined corresponding relative difference was obtained when the connective tissue of interest was healthy.

62. A method according to claim 61, wherein the echogenicity value of the connective tissue of interest obtained in step (i) is obtained after an injury to the first connective tissue.

63. A method according to claim 60, wherein the predetermined corresponding relative difference was obtained after an injury to the connective tissue of interest.

64. A method according to claim 63, wherein the echogenicity value of the connective tissue of interest obtained in step (i) is obtained while the injury to the first connective tissue is healing.

65. A method according to any of claims 55 to 58, wherein the second connective tissue in step (i) is healthy connective tissue, and the first and second connective tissue in step (iii) is healthy connective tissue, and wherein if the relative difference obtained in step (ii) is:
a) lower than the compared relative difference in step (iii), determining that the connective tissue of interest is injured or at risk of injury; or
b) similar to the compared relative difference in step (iii), determining that the connective tissue of interest is healthy; or
c) higher than the compared relative difference in step (iii), determining that the connective tissue of interest has high fitness potential.

66. A method according to any of claims 55 to 65, which further comprises repeating steps (i) to (iii) after a period to determine whether there is a change in the fitness potential of the connective tissue of interest.

67. A method according to claim 66, wherein the period is at least one day, at least a week, at least two weeks, or at least a month.

68. A method for determining a change in the fitness potential of a connective tissue of interest in a subject, the method comprising:
i) determining an echogenicity value of a first connective tissue in a limb of the subject, and an echogenicity value of a second connective tissue in the same limb of the subject, wherein the connective tissue of interest is the first connective tissue;
ii) determining a relative difference between the echogenicity values obtained for the first and second connective tissues in step (i);
iii) comparing the relative difference obtained in step (ii) with a corresponding relative difference obtained for first and second connective tissue in a limb of the same or a different subject;
iv) repeating steps (i) to (iii) after a period; and
v) determining whether there is a difference between the compared relative difference obtained in step (iii) and the compared relative difference obtained in step (iv); and if the compared relative difference obtained in step (iii) is:
a) higher than the compared relative difference obtained in step (iv), determining that the fitness potential of the connective tissue of interest has improved; or
b) similar to the compared relative difference obtained in step (iv), determining that the fitness potential of the connective tissue of interest has remained the same; or
c) lower than the compared relative difference obtained in step (iv), determining that the fitness potential of the connective tissue of interest has reduced.

69. A method according to claim 68, wherein the period is at least one day, at least a week, at least two weeks, or at least a month.

70. A method according to claim 68 or 69, wherein the second connective tissue in step (i) is healthy connective tissue, and the first and second connective tissue in step (iii) is healthy connective tissue.

71. A method according to claim 70, wherein the connective tissue of interest is an injured connective tissue, and if the compared relative difference obtained in step (iii) is higher than the compared relative difference obtained in step (iv), determining that the injury to the connective tissue of interest is healing.

72. A method according to claim 70, wherein the connective tissue of interest is a healthy connective tissue, and if the compared relative difference obtained in step (iii) is lower than the compared relative difference obtained in step (iv), determining that that the connective tissue of interest is at risk of injury.

73. A method according to any of claims 47 to 72, wherein the connective tissue is tendon or ligament tissue.

74. A method according to any of preceding claims 47 to 73, wherein the echogenicity values are determined quantitatively.

75. A method according to any of claims 47 to 74, wherein the echogenicity values are determined by ultrasonography.

76. A method according to claim 74 or 75, wherein the echogenicity values are determined by quantifying the echogenicity of ultrasonographic images.

77. A method according to claim 76, wherein the echogenicity is quantified by grey scale analysis of ultrasonographic images.

78. A method according to claim 77, which includes analysis of cross-sectional ultrasonographic images taken of the connective tissues.

79. A method according to claim 77 or 78, which includes analysis of longitudinal ultrasonographic images taken of the connective tissues.

80. A method according to any of claims 74 to 79, wherein a mean echogenicity value is determined for each connective tissue in each limb.

81. A method according to any of claims 47 to 80, wherein the connective tissue of interest comprises damaged tissue.

82. A method according to claim 81 wherein a mean echogenicity value is determined for a lesion in the damaged tissue.

83. A method according to claim 81, wherein a mean echogenicity value is determined for tissue excluding a lesion in the damaged tissue.

84. A method according to any of claims 81 to 83, wherein the tissue damage is the result of an injury to the subject.

85. A method according to claim 84, wherein the injury is an acute injury.

86. A method according to claim 84 or 85, wherein the injury is a traumatic injury.

87. A method according to claim 84, wherein the injury is an overload injury.

88. A method according to claim 84, wherein the injury is a chronic injury.

89. A method according to claim 88, wherein the chronic injury is caused by long-term micro-trauma or degradation of the connective tissue.

90. A method according to claim 88 or 89, wherein the chronic injury is a caused by chronic inflammation about the tissue.

91. A method according to any of claims 47 to 90, wherein the subject is a horse.

92. A method according to claim 91 wherein the subject is a polo horse.

93. A method according to claim 92, wherein the polo horse takes a right forelimb lead.

94. A method according to claim 91, wherein the subject is a racehorse.

95. A method according to claim 94, wherein the racehorse takes a right or a left forelimb lead.

96. A method according to any of claims 91 to 95, wherein the connective tissue comprises a flexor tendon or an extensor tendon.

97. A method according to claim 96, wherein the flexor tendon is a superficial digital flexor tendon (SDFT), or a deep digital flexor tendon (DDFT).

98. A method according to claim 97, wherein the first connective tissue is a SDFT and the second connective tissue is a DDFT.

99. A method according to claim 97, wherein the first connective tissue is a DDFT and the second connective tissue is a SDFT.

100. A method according to claim 96, wherein the extensor tendon is a lateral digital extensor tendon (LDET), or a common digital extensor tendon (CDET).

101. A method according to claim 100, wherein the first connective tissue is a LDET and the second connective tissue is a CDET.

102. A method according to claim 100, wherein the first connective tissue is a CDET and the second connective tissue is a LDET.

103. A method according to any of claims 91 to 95, wherein the connective tissue comprises a suspensory ligament, or a check ligament.

104. A method according to claim 103, wherein the first connective tissue is a suspensory ligament and the second connective tissue is a check ligament.

105. A method according to claim 103, wherein the first connective tissue is a check ligament and the second connective tissue is a suspensory ligament.

106. A method according to any of claims 47 to 90, wherein the subject is a non-human subject.

107. A method according to claim 106, wherein the first connective tissue is in a forelimb, or an upper limb, of the subject.

108. A method according to claim 106, wherein the first connective tissue is in a hindlimb, or a lower limb, of the subject.

109. A method according to any of claims 47 to 90, wherein the subject is a human subject.

110. A method according to claim 109, wherein the first connective tissue is in an upper limb of the subject.

111. A method according to claim 110, wherein the first connective tissue is a tendon in a finger in a hand of the subject, and the second connective tissue is a tendon in a different finger in the same hand of the subject.

112. A method according to claim 109, wherein the first connective tissue is in a lower limb of the subject.

113. A method according to claim 112, wherein the first connective tissue is a tendon in a toe in a foot of the subject, and the second connective tissue is a tendon in a different toe in the same foot of the subject.

114. A method according to claim 112, wherein the first connective tissue comprises a tendon in a foot of the subject, wherein the tendon is selected from a tibial tendon, an Achilles tendon, or a peroneal tendon, and the second connective tissue comprises a different tendon in the same foot of the subject, wherein the tendon is selected from a tibial tendon, an Achilles tendon, or a peroneal tendon.

115. A method according to claim 112, wherein the first connective tissue comprises a posterior tibial tendon, and the second connective tissue comprises an anterior tibial tendon.

116. A method according to claim 112, wherein the first connective tissue comprises an anterior tibial tendon, and the second connective tissue is a posterior tibial tendon.

117. A method for determining the fitness potential of a connective tissue of interest in a subject, the method comprising:
i) determining an echogenicity value of the connective tissue of interest in a first limb of the subject;
ii) determining an echogenicity value of an equivalent connective tissue in a second limb of the subject;
iii) determining a ratio of the echogenicity value of the connective tissue of interest in the first limb to the echogenicity value of the equivalent connective tissue in the second limb; and
iv) if the ratio determined in step (iii) is:
a) less than one, determining that the fitness potential of the connective tissue of interest in the first limb is lower than the fitness potential of the equivalent connective tissue in the second limb; or
b) greater than one, determining that the fitness potential of the connective tissue of interest in the first limb is higher than the fitness potential of the equivalent connective tissue in the second limb; or
c) one, determining that the fitness potential of the connective tissue of interest in the first limb is the same as the fitness potential of the equivalent connective tissue in the second limb.

118. A method according to claim 117, wherein the connective tissue in the second limb is healthy, and if the ratio determined in step (iii) is:
a) less than one, determining that the connective tissue of interest is injured or at risk of injury; or
b) greater than one, determining that the connective tissue of interest has a high fitness potential; or
c) one, determining that the connective tissue of interest is healthy.

119. A method according to claim 117 or 118, which further comprises repeating steps (i) to (iii) after a period to determine whether there is a change in the fitness potential of the connective tissue of interest.

120. A method according to claim 119, wherein the period is at least one day, at least a week, at least two weeks, or at least a month.

121. A method for determining a change in the fitness potential of a connective tissue of interest in a subject, the method comprising:
i) determining an echogenicity value of a connective tissue of interest in a first limb of the subject;
ii) determining an echogenicity value of an equivalent connective tissue in a second limb of the subject;
iii) determining a ratio of the echogenicity value of the connective tissue of interest in the first limb to the echogenicity value of the connective tissue in the second limb;
iv) repeating steps (i) to (iii) after a period; and
v) if the ratio obtained in step (iv) is:
a) higher than the ratio obtained in step (iii), determining that the fitness potential of the connective tissue of interest has increased; or
b) the same as the ratio obtained in step (iii), determining that the fitness potential of the connective tissue of interest has remained the same; or
c) lower than the ratio obtained in step (iii), determining that the fitness potential of the connective tissue of interest has reduced.

122. A method according to claim 121, wherein the connective tissue of interest is an injured connective tissue, and if the ratio obtained in step (iv) is higher than the ratio obtained in step (iii), it is determined that the injury to the connective tissue of interest is healing.

123. A method according to claim 121, wherein the connective tissue of interest is a healthy connective tissue, and if the ratio obtained in step (iv) is lower than the ratio obtained in step (iii), it is determined that the connective tissue of interest is at risk of injury.

124. A method according to any of claims 117 to 123, wherein the second limb is a different limb of the same subject.

125. A method according to any of claims 117 to 124, wherein the echogenicity value of the equivalent connective tissue in the second limb determined in step (ii) is a predetermined echogenicity value previously obtained for the connective tissue of the compared limb.

126. A method according to claim 125, wherein the predetermined echogenicity value was obtained when the connective tissue of the compared limb was healthy.

127. A method according to any of claims 117 to 126, wherein the echogenicity value of the connective tissue of interest obtained in step (i) is obtained after an injury to the connective tissue of interest.

128. A method according to claim 127, wherein the echogenicity value of the connective tissue of interest obtained in step (i) is obtained while the injury to the connective tissue of interest is healing.

129. A method according to any of claims 117 to 128, wherein the subject is a non-human subject.

130. A method according to claim 129, wherein the connective tissue of interest is in a forelimb, or an upper limb, of the subject, and the equivalent connective tissue is in the opposite forelimb, or opposite upper limb, of the subject.

131. A method according to claim 129, wherein the connective tissue of interest is in a hindlimb, or a lower limb, of the subject, and the equivalent connective tissue is in the opposite hindlimb, or opposite lower limb, of the subject.

132. A method according to any of claims 117 to 128, wherein the subject is a human subject.

133. A method according to claim 132, wherein the connective tissue of interest is in an upper limb of the subject, and the equivalent connective tissue is in the opposite upper limb of the subject.

134. A method according to claim 132, wherein the connective tissue of interest is in a lower limb of the subject, and the equivalent connective tissue is in the opposite lower limb of the subject.

135. A method according to any of claims 117 to 134, wherein the connective tissue is tendon or ligament tissue.

136. A method according to claim 135, wherein the connective tissue is a flexor tendon or an extensor tendon.

137. A method according to claim 135 or 136, wherein the first connective tissue is a tendon in a finger in a hand of the subject, and the second connective tissue is a tendon in a corresponding finger in the opposite hand of the subject.

138. A method according to claim 135 or 136, wherein the first connective tissue is a tendon in a toe in a foot of the subject, and the second connective tissue is a tendon in a corresponding toe in the opposite foot of the subject.

139. A method according to claim 132, wherein the first connective tissue comprises a tendon in a foot of the subject, wherein the tendon is selected from a tibial tendon, an Achilles tendon, or a peroneal tendon, and the second connective tissue comprises a corresponding tendon in the opposite foot of the subject.

140. A method according to any of claims 117 to 139, wherein the echogenicity values are determined quantitatively.

141. A method according to any of claims 117 to 140, wherein the echogenicity values are determined by ultrasonography.

142. A method according to claim 140 or 141, wherein the echogenicity values are determined by quantifying the echogenicity of ultrasonographic images.

143. A method according to claim 142, wherein the echogenicity is quantified by grey scale analysis of ultrasonographic images.

144. A method according to claim 143, which includes analysis of cross-sectional ultrasonographic images taken of the connective tissues.

145. A method according to claim 143 or 144, which includes analysis of longitudinal ultrasonographic images taken of the connective tissues.

146. A method according to any of claims 140 to 145, wherein a mean echogenicity value is determined for the connective tissue in the first limb, and a mean echogenicity value is determined for the connective tissue in the second limb.

147. A method according to any of claims 117 to 146, wherein the connective tissue of interest comprises damaged tissue.

148. A method according to claim 147, wherein a mean echogenicity value is determined for a lesion in the damaged tissue.

149. A method according to claim 148, wherein a mean echogenicity value is determined for tissue excluding a lesion in the damaged tissue.

150. A method according to any of claims 147 to 149, wherein the tissue damage is the result of an injury to the subject.

151. A method according to claim 150, wherein the injury is an acute injury.

152. A method according to claim 150 or 151, wherein the injury is a traumatic injury.

153. A method according to claim 150, wherein the injury is an overload injury.

154. A method according to claim 150, wherein the injury is a chronic injury.

155. A method according to claim 154, wherein the chronic injury is caused by long-term micro-trauma or degradation of the connective tissue.

156. A method according to claim 154 or 155, wherein the chronic injury is a caused by chronic inflammation about the tissue.

157. A method according to any of claims 47 to 97, 103, 106 to 110, or 112, wherein the first connective tissue comprises tendon tissue.

158. A method according to any of claims 47 to 97, 103, 106 to 110, 112, or 157, wherein the second connective tissue comprises ligament tissue.

159. A method according to any of claims 47 to 97, 103, 106 to 110, 112, 157 or 158, wherein the first connective tissue comprises tendon tissue, and the second connective tissue comprises ligament tissue.

160. A method according to any of claims 47 to 97, 103, 106 to 110, or 112, wherein the first connective tissue comprises ligament tissue.

161. A method according to any of claims 47 to 97, 103, 106 to 110, 112, or 160, wherein the second connective tissue comprises tendon tissue.

162. A method according to any of claims 47 to 97, 103, 106 to 110, 112, 160 or 161, wherein the first connective tissue comprises ligament tissue, and the second connective tissue comprises tendon tissue.

163. A method for determining the fitness potential of an area of interest in a tissue of a subject, the method comprising:
i) determining an echogenicity value of a first area of the tissue, and an echogenicity value of a second area of the same tissue spatially distant from the first area, wherein the area of interest is the first area;
ii) determining a relative difference of the echogenicity value of the first area to the echogenicity value of the second area; and
iii) if the relative difference determined in step (ii) is:
a) less than one, determining that the fitness potential of the area of interest is lower than the fitness potential of the second area;
b) greater than one, determining that the fitness potential of the area of interest is higher than the fitness potential of the second area; or
c) one, determining that the fitness potential of the area of interest is the same as the fitness potential of the second area.

164. A method according to claim 163, wherein the second area is healthy tissue, and in step (iii), if the relative difference determined in step (ii) is:
a) less than one, determining that the area of interest is injured or at risk of injury; or
b) greater than one, determining that the area of interest has a high fitness potential; or
c) one, determining that the area of interest is healthy.

165. A method according to claim 163 or 164, which further comprises repeating steps (i) to (iii) after a period to determine whether there is a change in the fitness potential of the area of interest.

166. A method according to claim 165, wherein the period is at least one day, at least a week, at least two weeks, or at least a month.

167. A method for determining a change in the fitness potential of an area of interest in tissue in a subject, the method comprising:
i) determining an echogenicity value of a first area of the tissue, and an echogenicity value of a second area of the same tissue spatially distant from the first area, wherein the area of interest is the first area of the tissue;
ii) determining a relative difference of the echogenicity value of the first area to the echogenicity value of the second area;
iii) repeating steps (i) and (ii) after a period; and
iv) if the relative difference obtained in step (iii) is:
a) higher than the relative difference obtained in step (ii), determining that the fitness potential of the area of interest has increased; or
b) similar to the relative difference obtained in step (ii), determining that the fitness potential of the area of interest has remained the same; or
c) lower than the relative difference obtained in step (ii), determining that the fitness potential of the area of interest has reduced.

168. A method according to claim 167, wherein the period is at least one day, at least a week, at least two weeks, or at least a month.

169. A method according to claim 167 or 168, wherein the area of interest comprises injured tissue, and if the relative difference obtained in step (iii) is higher than the relative difference obtained in step (ii), it is determined that the injury to the area of interest is healing.

170. A method according to claim 167 or 168, wherein the area of interest is healthy tissue, and if the relative difference obtained in step (iii) is lower than the relative difference obtained in step (ii), it is determined that the area of interest is at risk of injury.

171. A method according to any of claims 163 to 170, further comprising:
a) comparing the relative difference obtained in step (ii) with a relative difference obtained for a corresponding first and second area for tissue of the same type as for the relative difference obtained in step (ii) but at the same or a different site for the same subject, or at the same or a different site for a different subject; and
b) if the relative difference obtained in step (ii) is:
lower than the relative difference obtained in step (a), determining that the fitness potential of the area of interest is lower than the fitness potential of the corresponding first area of tissue in the compared tissue; or
similar to the relative difference obtained in step (a), determining that the fitness potential of the area of interest is similar to the fitness potential of the corresponding first area of tissue in the compared tissue; or
higher than the relative difference obtained in step (a), determining that the fitness potential of the area of interest is higher than the fitness potential of the corresponding first area of tissue in the compared tissue.

172. A method according to claim 171, wherein the corresponding first and second area in step (a) is at the same site for a different subject.

173. A method according to claim 171, wherein the corresponding first and second area in step (a) is at a different site for a different subject.

174. A method according to claim 171, wherein the corresponding first and second area in step (a) is at a same site for the same subject.

175. A method according to claim 171, wherein the corresponding first and second area in step (a) is at a different site for the same subject.

176. A method according to any of claims 171 to 175 wherein the relative difference obtained for the corresponding first and second area of the compared tissue in step (a) is a predetermined relative difference previously obtained for the first and second area of the compared tissue.

177. A method according to claim 176, wherein the predetermined relative difference was obtained when the area of interest was healthy.

178. A method according to claim 177, wherein the echogenicity value of the area of interest obtained in step (i) is obtained after an injury to the area of interest.

179. A method according to claim 176, wherein the predetermined relative difference was obtained after an injury to the area of interest.

180. A method according to claim 179, wherein the echogenicity value of the area of interest obtained in step (i) is obtained while the injury to the area of interest is healing.

181. A method according to any of claims 171 to 175, wherein the second area of tissue in step (i) is healthy tissue, and the first and second area of tissue in step (a) is healthy tissue, and wherein if the relative difference obtained in step (ii) is:
a) lower than the compared relative difference in step (a), determining that the area of interest is injured or at risk of injury; or
b) similar to the compared relative difference in step (a), determining that the area of interest is healthy; or
c) higher than the compared relative difference in step (a), determining that the area of interest has high fitness potential.

182. A method according to any of claims 171 to 181, which further comprises repeating steps (i) to (iii) after a period to determine whether there is a change in the fitness potential of the area of interest.

183. A method according to claim 182, wherein the period is at least one day, at least a week, at least two weeks, or at least a month.

184. A method for determining a change in the fitness potential of an area of interest in a tissue of a subject, the method comprising:
i) determining an echogenicity value of a first area of the tissue, and an echogenicity value of a second area of the same tissue spatially distant from the first area, wherein the area of interest is the first area;
ii) determining a relative difference between the echogenicity values obtained for the first and second areas in step (i);
iii) comparing the relative difference obtained in step (ii) with a relative difference obtained for a corresponding first and second area of the same tissue of the same or different subject;
iv) repeating steps (i) to (iii) after a period; and
v) determining whether there is a difference between the compared relative difference obtained in step (iii) and the compared relative difference obtained in step (iv); and if the compared relative difference obtained in step (iii) is:
a) higher than the compared relative difference obtained in step (iv), determining that the fitness potential of the area of interest has improved; or
b) similar to the compared relative difference obtained in step (iv), determining that the fitness potential of the area of interest has remained the same; or
c) lower than the compared relative difference obtained in step (iv), determining that the fitness potential of the area of interest has reduced.

185. A method according to claim 184, wherein the period is at least one day, at least a week, at least two weeks, or at least a month.

186. A method according to claim 184 or 185, wherein the second area in step (i) is healthy tissue, and the first and second areas in step (iii) are healthy connective tissues.

187. A method according to claim 186, wherein the area of interest comprises injured tissue, and if the compared relative difference obtained in step (iii) is higher than the compared relative difference obtained in step (iv), determining that the injury to the area of interest is healing.

188. A method according to claim 186, wherein the area of interest is healthy tissue, and if the compared relative difference obtained in step (iii) is lower than the compared relative difference obtained in step (iv), determining that that the area of interest is at risk of injury.

189. A method according to any of claims 163 to 188, wherein the tissue comprises connective tissue.

190. A method according to any of claims 163 to 189, wherein the tissue comprises tendon or ligament tissue.

191. A method according to any of claims 163 to 188, wherein the tissue comprises muscle tissue.

192. A method according to any of claims 163 to 188, or 191, wherein the tissue comprises cardiac muscle tissue.

193. A method for determining the fitness potential of a muscle tissue of interest, wherein the method comprises:
i) determining an echogenicity value of a muscle tissue of interest; and
ii) if the echogenicity value obtained in (i) is:
a) less than an echogenicity value for a corresponding muscle tissue, determining that the fitness potential of the tissue of interest is lower than the fitness potential of the corresponding muscle tissue;
b) greater than an echogenicity value for the corresponding muscle tissue, determining that the fitness potential of the tissue of interest is higher than the fitness potential of the corresponding muscle tissue; or
c) the same as an echogenicity value for the corresponding muscle tissue, determining that the fitness potential of the tissue of interest is the same as the fitness potential of the corresponding muscle tissue.

194. A method according to claim 193, wherein the corresponding muscle tissue is healthy tissue, and in step (ii), if the echogenicity value determined in step (i) is:
a) less than an echogenicity value for the corresponding muscle tissue, determining that the tissue of interest is injured or at risk of injury;
b) greater than an echogenicity value for the corresponding muscle tissue, determining that the tissue of interest has high fitness potential; or
c) the same as an echogenicity value for the corresponding muscle tissue, determining that the tissue of interest is healthy.

195. A method according to claim 193 or 194, which further comprises repeating steps (i) and (ii) after a period to determine whether there is a change in the fitness potential of the tissue of interest.

196. A method according to claim 195, wherein the period is at least one day, at least a week, at least two weeks, or at least a month.

197. A method according to any of claims 193 to 196, wherein the corresponding muscle tissue is the same muscle tissue of the same subject.

198. A method according to any of claims 193 to 197, wherein the echogenicity value of the corresponding muscle tissue is a predetermined value previously obtained.

199. A method according to claim 198, wherein the echogenicity value of the corresponding muscle tissue is a predetermined value previously obtained from the same subject.

200. A method according to claim 199, wherein the predetermined echogenicity value was obtained when the muscle tissue of interest was healthy.

201. A method according to any of claims 193 to 200, wherein the echogenicity value of the muscle tissue of interest obtained in step (i) was obtained after an injury to the muscle tissue.

202. A method according to any of claims 193 to 196, wherein the corresponding muscle tissue is the same muscle tissue of a different subject.

203. A method according to claim 202, wherein the echogenicity value of the corresponding muscle tissue is a predetermined echogenicity value obtained from a different subject.

204. A method according to claim 202 or 203, wherein the echogenicity value of the corresponding muscle tissue was obtained after an injury to the muscle tissue of interest.

205. A method according to claim 204, wherein the echogenicity of the muscle tissue of interest obtained in step (i) is obtained while the injury to the muscle tissue of interest is healing.

206. A method for determining a change in the fitness potential of a muscle tissue of interest in a subject, the method comprising:
i) determining an echogenicity value of the muscle tissue of interest;
ii) repeating step (i) after a period; and
iii) if the value obtained in step (ii) is:
a) higher than the value obtained in step (i), determining that the fitness potential of the muscle tissue of interest has increased; or
b) similar to the value obtained in step (i), determining that the fitness potential of the muscle tissue of interest has remained the same; or
c) lower than the value obtained in step (i), determining that the fitness potential of the muscle tissue of interest has reduced.

207. A method according to claim 206, wherein the period is at least one day, at least a week, at least two weeks, or at least a month.

208. A method according to claim 206 or 207, wherein the muscle tissue of interest is an injured tissue, and if the value obtained in step (ii) is higher than the value obtained in step (i), it is determined that the injury to the tissue of interest is healing.

209. A method according to claim 206 or 207, wherein the muscle tissue of interest is a healthy tissue, and if the value obtained in step (iii) is lower than the value obtained in step (ii), it is determined that the tissue of interest is at risk of injury.

210. A method according to any of claims 193 to 209, wherein the muscle tissue is cardiac muscle tissue.

211. A method according to any of claims 163 to 210, wherein the echogenicity values are determined quantitatively.

212. A method according to any of claims 163 to 211, wherein the echogenicity values are determined by ultrasonography.

213. A method according to claim 211 or 212, wherein the echogenicity values are determined by quantifying the echogenicity of ultrasonographic images.

214. A method according to claim 213, wherein the echogenicity is quantified by grey scale analysis of ultrasonographic images.

215. A method according to claim 214, which includes analysis of cross-sectional ultrasonographic images taken of the tissues.

216. A method according to claim 214 or 215, which includes analysis of longitudinal ultrasonographic images taken of the tissues.

217. A method according to any of claims 211 to 216, wherein a mean echogenicity value is determined for each tissue.

218. A method according to any of claims 193 to 217, wherein the tissue of interest comprises damaged tissue.

219. A method according to claim 218, wherein a mean echogenicity value is determined for a lesion in the damaged tissue.

220. A method according to claim 218, wherein a mean echogenicity value is determined for tissue excluding a lesion in the damaged tissue.

221. A method according to any of claims 218 to 220, wherein the tissue damage is the result of an injury to the subject.

222. A method according to claim 221, wherein the injury is an acute injury.

223. A method according to claim 222, wherein the muscle tissue is cardiac muscle tissue, and the injury is caused by decreased or complete cessation of blood flow to the cardiac tissue.

224. A method according to claim 223, wherein the injury is caused by a full or partial occlusion of a blood vessel.

225. A method according to claim 224, wherein the blood vessel is a coronary artery.

226. A method according to claim 222, wherein the injury is caused by a tear or strain.

227. A method according to claim 226, wherein the muscle is cardiac muscle and the injury is caused by blunt cardiac injury.

228. A method according to claim 221 or 222, wherein the injury is a traumatic injury.

229. A method according to claim 221, wherein the injury is an overload injury.

230. A method according to claim 221, wherein the injury is a chronic injury.

231. A method according to claim 230, wherein the injury is caused by long-term disruption of blood flow to the tissue.

232. A method according to claim 231, wherein the tissue is cardiac tissue and the injury is caused by atherosclerosis.

233. A method according to claim 230, wherein the chronic injury is caused by long-term micro-trauma or degradation of the connective tissue.

234. A method according to claim 230 or 233, wherein the chronic injury is a caused by chronic inflammation about the tissue.

235. A method for determining a size of a lesion in a muscle tissue, which comprises determining an area of the lesion from an ultrasonogram of an ultrasound scan carried out on the muscle tissue.

236. A method according to claim 235, wherein a treatment program is initiated for the subject which is appropriate for the size and/or severity of the lesion.

237. A method according to claim 236, wherein the treatment program comprises administering to the subject anti-inflammatory medication, dietary supplementation, or stem-cell treatment, and/or cold therapy, rest, confinement, surgery, or a loading/working training regime.

238. A method according to any of claims 235 to 237, which comprises repeating the method after a period to determine whether there is a change in the area of the lesion.

239. A method according to claim 238, wherein the period is at least one day, at least a week, at least two weeks, at least a month, at least six months, or at least twelve months.

240. A method according to claim 238 or 239, wherein if it is determined that the area of the lesion has decreased over the period, determining that the lesion is healing.

241. A method according to claim 238 or 239, wherein if it is determined that the area of the lesion has increased over the period, determining that the lesion is progressing.

242. A method for monitoring a change in a size of a lesion in a muscle tissue, which comprises:
i) determining an area of the lesion from an ultrasonogram of an ultrasound scan carried out on the muscle tissue;
ii) repeating step (i) after a period; and
iii) determining whether the area of the lesion has changed.

243. A method according to claim 242, wherein if it is determined that the area of the lesion obtained in step (iii) is less than that obtained in step (ii), determining that the lesion size has decreased over the period.

244. A method according to claim 243, wherein it is determined that the lesion is healing.

245. A method according to claim 242, wherein if it is determined that the area of the lesion obtained in step (iii) is greater than that obtained in step (ii), determining that the lesion size has increased over the period.

246. A method according to claim 245, wherein it is determined that the lesion is progressing.

247. A method according to claim 242, wherein if it is determined that the area of the lesion obtained in step (iii) is the same as that obtained in step (ii), determining that the lesion size has remained the same over the period.

248. A method according to claim 247, wherein it is determined that the lesion is stable.

249. A method according to any of claims 242 to 248, wherein the period is at least one day, at least a week, at least two weeks, at least a month, at least six months, or at least twelve months.

250. A method according to any of claims 242 to 249, which further comprises carrying out the ultrasound scan of the tissue, and providing an ultrasonogram from the ultrasound scan.

251. A method according to claim 250, wherein the ultrasonogram is a cross-sectional ultrasonogram.

252. A method according to claim 250, wherein the ultrasonogram is a longitudinal ultrasonogram.

253. A method according to any of claims 242 to 252, wherein the area of the lesion is determined by grey scale analysis of the ultrasonograms.

254. A method according to any of claims 235 to 253, wherein an area of the muscle tissue which does not form a part of the area of the lesion is healthy muscle tissue.

255. A method according to any of claims 235 to 254, wherein the muscle tissue is cardiac tissue.

256. A method according to any of claims 235 to 255, wherein the lesion is the result of an injury to the subject.

257. A method according to claim 256, wherein the injury is an acute injury.

258. A method according to claim 257, wherein the muscle tissue is cardiac muscle tissue, and the injury is caused by decreased or complete cessation of blood flow to the cardiac tissue.

259. A method according to claim 258, wherein the injury is caused by a full or partial occlusion of a blood vessel.

260. A method according to claim 259, wherein the blood vessel is a coronary artery.

261. A method according to claim 257, wherein the injury is caused by a tear or strain.

262. A method according to claim 261, wherein the muscle is cardiac muscle and the injury is caused by blunt cardiac injury.

263. A method according to claim 256 or 257, wherein the injury is a traumatic injury.

264. A method according to claim 256, wherein the injury is an overload injury.

265. A method according to claim 256, wherein the injury is a chronic injury.

266. A method according to claim 265, wherein the injury is caused by long-term disruption of blood flow to the tissue.

267. A method according to claim 266, wherein the tissue is cardiac tissue and the injury is caused by atherosclerosis.

268. A method according to claim 265, wherein the chronic injury is caused by long-term micro-trauma or degradation of the muscle tissue.

269. A method according to claim 265 or 268, wherein the chronic injury is caused by chronic inflammation about the tissue.
